(19) United States
(12) Patent Application Publication
(10) Pub. No.: US 2024/0197416 A1
(43) Pub. Date: Jun. 20, 2024

(54) HUB SENSING THROUGH A STERILE BARRIER IN A ROBOTIC CATHETER ASSEMBLY

(71) Applicant: Imperative Care, Inc., Campbell, CA (US)

(72) Inventors: David Gonzalez, Campbell, CA (US); Kyle Bartholomew, Campbell, CA (US); Lilip Lau, Los Altos, CA (US)

(21) Appl. No.: 18/389,628

(22) Filed: Dec. 19, 2023

Related U.S. Application Data
(63) Continuation-in-part of application No. 17/527,393, filed on Nov. 16, 2021.
(63) Continuation-in-part of application No. 17/527,379, filed on Nov. 16, 2021.
(63) Continuation-in-part of application No. 17/527,460, filed on Nov. 16, 2021.
(63) Continuation-in-part of application No. 17/527,452, filed on Nov. 16, 2021.
(63) Continuation-in-part of application No. 17/527,456, filed on Nov. 16, 2021.
(63) Continuation-in-part of application No. 18/073,291, filed on Dec. 1, 2022.
Application No. 18/073,291 is a continuation-in-part of application No. 17/960,014, filed on Oct. 4, 2022.
Application No. 17/960,014 is a continuation-in-part of application No. 17/816,669, filed on Aug. 1, 2022.
Application No. 18/073,291 is a continuation-in-part of application No. 17/959,894, filed on Oct. 4, 2022.
Application No. 17/959,894 is a continuation-in-part of application No. 17/816,669, filed on Aug. 1, 2022.
Application No. 18/073,291 is a continuation-in-part of application No. 17/959,924, filed on Oct. 4, 2022.
Application No. 17/959,924 is a continuation-in-part of application No. 17/816,669, filed on Aug. 1, 2022.
(63) Continuation-in-part of application No. 18/060,935, filed on Dec. 1, 2022.
Application No. 18/060,935 is a continuation-in-part of application No. 17/960,014, filed on Oct. 4, 2022.
Application No. 17/960,014 is a continuation-in-part of application No. 17/959,894, filed on Oct. 4, 2022.
Application No. 17/959,894 is a continuation-in-part of application No. 17/959,924, filed on Oct. 4, 2022.
(60) Provisional application No. 63/434,033, filed on Dec. 20, 2022.
(60) Provisional application No. 63/232,444, filed on Aug. 12, 2021.
(60) Provisional application No. 63/232,444, filed on Aug. 12, 2021.
(60) Provisional application No. 63/232,444, filed on Aug. 12, 2021.
(60) Provisional application No. 63/232,444, filed on Aug. 12, 2021.
(60) Provisional application No. 63/232,444, filed on Aug. 12, 2021.

(51) Int. Cl. A61B 34/30; A61B 34/00; G01R 33/02

(52) U.S. Cl. CPC A61B 34/30; A61B 34/25; G01R 33/02; A61B 2034/301

(57) ABSTRACT

A robotic drive system includes a hub configured to adjust an axial position of an interventional device, a driven magnet coupled with the hub, a hub adapter configured to move in at least one direction based on an input provided by a user of the robotic control system, a drive magnet coupled with the hub adapter and configured to couple with the driven magnet such that the driven magnet moves in response to movement of the drive magnet, and a sensor coupled with the hub or the hub adapter and configured to measure a magnitude of a magnetic field on the sensor from one or both of the drive magnet and the driven magnet.

### PRIORITY APPLICATIONS

Any and all applications for which a foreign or domestic priority claim is identified in the Application Data Sheet as filed with the present application are hereby incorporated by reference under 37 CFR 1.57.

This application claims the priority benefit under 35 U.S.C. § 119(c) of U.S. Provisional Patent Application No. 63/434,033, filed Dec. 20, 2022.

This application is also a continuation-in-part of U.S. application Ser. No. 17/527,393, filed Nov. 16, 2021, which claims the priority benefit under 35 U.S.C. § 119(c) of U.S. Provisional Patent Application No. 63/232,444, filed Aug. 12, 2021. This application is also a continuation-in-part of U.S. patent application Ser. No. 17/527,379, filed Nov. 16, 2021, which claims the priority benefit under 35 U.S.C. § 119(c) of U.S. Provisional Patent Application No. 63/232,444, filed Aug. 12, 2021. This application is also a continuation-in-part of U.S. patent application Ser. No. 17/527,460, filed Nov. 16, 2021, which claims the priority benefit under 35 U.S.C. § 119(c) of U.S. Provisional Patent Application No. 63/232,444, filed Aug. 12, 2021. This application is also a continuation-in-part of U.S. patent application Ser. No. 17/527,452, filed Nov. 16, 2021, which claims the priority benefit under 35 U.S.C. § 119(c) of U.S. Provisional Patent Application No. 63/232,444, filed Aug. 12, 2021. This application is also a continuation-in-part of U.S. patent application Ser. No. 17/527,456, filed Nov. 16, 2021, which claims the priority benefit under 35 U.S.C. § 119(c) of U.S. Provisional Patent Application No. 63/232,444, filed Aug. 12, 2021.

This application is also a continuation-in-part of U.S. application Ser. No. 18/073,291, filed Dec. 1, 2022, which is a continuation-in-part of U.S. application Ser. No. 17/960,014, filed Oct. 4, 2022, which is a continuation-in-part of U.S. application Ser. No. 17/816,669, filed Aug. 1, 2022. U.S. application Ser. No. 18/073,291 is also a continuation-in-part of U.S. application Ser. No. 17/959,894, filed Oct. 4, 2022, which is a continuation-in-part of U.S. application Ser. No. 17/816,669, filed Aug. 1, 2022. U.S. application Ser. No. 18/073,291 is also a continuation-in-part of U.S. application Ser. No. 17/959,924, filed Oct. 4, 2022, which is a continuation-in-part of U.S. application Ser. No. 17/816,669, filed Aug. 1, 2022. This application is also a continuation-in-part of U.S. application Ser. No. 18/060,935, filed Dec. 1, 2022, which is a continuation-in-part of U.S. application Ser. No. 17/960,014, filed Oct. 4, 2022, which is a continuation-in-part of U.S. application Ser. No. 17/816,669, filed Aug. 1, 2022. U.S. application Ser. No. 18/060,935 is also a continuation-in-part of U.S. application Ser. No. 17/959,894, filed Oct. 4, 2022, which is a continuation-in-part of U.S. application Ser. No. 17/816,669, filed Aug. 1, 2022. U.S. application Ser. No. 18/060,935 is also a continuation-in-part of U.S. application Ser. No. 17/959,924, filed Oct. 4, 2022, which is a continuation-in-part of U.S. application Ser. No. 17/816,669, filed Aug. 1, 2022.

The disclosures of all these prior applications are hereby incorporated herein by reference in their entireties and are to be to be considered a part of this specification.

### FIELD

The present application relates to neurovascular procedures, and more particularly, to catheter assemblies and robotic control systems for neurovascular site access.

### BACKGROUND

A variety of neurovascular procedures can be accomplished via a transvascular access, including thrombectomy, diagnostic angiography, embolic coil deployment and stent placement. However, the delivery of neurovascular care is limited or delayed by a variety of challenges. For example, there are not enough trained interventionalists and centers to meet the current demand for neuro interventions. Neuro interventions are difficult, with complex set up requirements and demands on the surgeon's dexterity. With two hands, the surgeon must exert precise control over 3-4 coaxial catheters plus manage the fluoroscopy system and patient position. Long, tortuous anatomy, requires delicate, precise maneuvers. Inadvertent catheter motion can occur due energy storage and release caused by frictional interplay between coaxial shafts and the patient's vasculature. Supra-aortic access necessary to reach the neurovasculature is challenging to achieve, especially Type III arches. Once supra-aortic access is achieved, adapting the system for neurovascular treatments is time consuming and requires guidewire and access catheter removal and addition of a procedure catheter (and possibly one or more additional catheters) to the stack.

Thus, there remains a need for a supra-aortic access and neurovascular site access system that addresses some or all these challenges and increases the availability of neurovascular procedures. Preferably, the system is additionally capable of driving devices further distally through the supra-aortic access to accomplish procedures in the intracranial vessels.

### SUMMARY

Disclosed herein are embodiments of a robotic control system for interventional treatments. Some embodiments of the robotic control system disclosed herein can use a hub adapter to move a hub positioned on a sterile side of a sterile barrier, wherein one or more interventional devices are coupled with the hub. In some embodiments, a magnetic field can be created between the hub adapter and the hub such that a movement of the hub adapter results in a movement of the hub as a result of the magnetic field between the hub and hub. In some embodiments, magnetic coupling can be used in any embodiments of the robotic control systems disclosed herein so that a movement of a driving device on a non-sterile side of a sterile barrier can cause a movement of at least one device on the sterile side of the sterile barrier in any desired direction of movement (e.g., in the direction of insertion and/or withdrawal of an interventional device relative to a port going into a patient's body). The magnetic force between the hub adapter and the hub of some embodiments is elastic.

In some embodiments, one or more sensors can measure a magnetic field strength and/or direction of a magnetic coupling between a hub and a hub adapter. The magnetic field strength and/or direction can be used to determine relative displacement of the hub from the hub adapter, detachment of the hub from the hub adapter, and/or an axial load acting on the hub.

In some embodiments, the relative displacement of a hub with respect to a corresponding hub adapter can be observed, measured, and characterized using one or more sensors of some embodiments of the system disclosed herein. For example, some embodiments can include a magnetometer device in the hub adapter or hub and a corresponding magnet in the other of the hub adapter and the hub. The relative displacement of hub and hub adapter can be characterized with changes in the magnetic field strength and/or direction detected by a magnetometer. The information of the magnetic field strength and/or direction generated by the magnet opposite to a magnetometer can be used in some embodiments to measure either the vertical or horizontal displacement of the hub relative to the hub adapter. Additionally, in some embodiments, the magnetic field strength and/or direction data can be used to calculate the force acting on hub from the magnetic coupling and/or a separate external force acting on the hub.

Disclosed herein are embodiments of a robotic control system for interventional treatments (also referred to herein as a robotic system for interventional treatments). In any embodiments disclosed herein, the system can include a hub that can be configured to adjust an axial position of an interventional device, a driven magnet coupled with the hub, a hub adapter that can be configured to move in at least one direction based on an input provided by a user of the robotic control system, a drive magnet coupled with the hub adapter and configured to couple with the driven magnet such that the driven magnet moves in response to movement of the drive magnet, and a sensor coupled with the hub or the hub adapter and configured to measure a magnitude of a magnetic field on the sensor.

Any embodiments of the methods, devices and systems disclosed herein can include, in additional embodiments, one or more of the following steps, features, components, and/or details, in any combination with any of the other steps, features, components, and/or details of any other embodiments disclosed herein: wherein the drive magnet and driven magnet magnetically couple the hub adapter to the hub when the hub is within a predetermined distance of the hub adapter in the at least one direction; wherein the driven magnet biases the hub to remain in approximate alignment with the hub adapter in the at least one direction when the hub adapter moves in the at least one direction; wherein the sensor is a magnetometer; wherein the robotic control system is configured to determine a relative distance in the at least one direction between the hub adapter and the hub when the hub is at least partially offset from the hub adapter in the at least one direction based on the measured magnitude of the magnetic field on the sensor; wherein the robotic control system is configured to provide a warning when the relative distance reaches or exceeds a threshold value; wherein the robotic control system is configured to determine a magnitude of a net external force acting on the hub in the at least one direction based on the measured magnitude of the magnetic field on the sensor; wherein the robotic control system is configured to determine a relative distance in the at least one direction between the hub adapter and the hub when the hub is at least partially offset from the hub adapter in the at least one direction based on the measured magnitude of the magnetic field on the sensor, and/or wherein the robotic control system is configured to determine a magnitude of a net external force acting on the hub in the at least one direction based at least in part on the relative distance in the at least one direction between the hub adapter and the hub when the hub is offset from the hub adapter in the at least one direction; wherein the robotic control system is configured to provide an alert when the external force in the at least one direction reaches or exceeds a threshold value; wherein the robotic control system is configured to automatically implement a correction action when the external force in the at least one direction reaches or exceeds a threshold value; wherein the correction action comprises stopping any movement of any hubs or hub adapters, stopping any movement of any hubs or hub adapters in any direction which would increase the external force, unloading one or more catheters or other devices to reduce the external force, providing information to a user of the system to assist the user in reducing the external force, and/or providing specific instructions to the user to instruct the user on maneuvers that would reduce the external force; wherein the hub is coupled to the hub adapter across a sterile barrier; wherein the hub is positioned on a sterile side of the sterile barrier and the hub adapter is positioned on a nonsterile side of the sterile barrier; wherein the sensor is coupled with the hub adapter; wherein the interventional device is a guide catheter, a procedure catheter, an access catheter, or a guidewire; wherein the interventional device is an aspiration catheter, an embolic deployment catheter, a stent deployment catheter, a flow diverter deployment catheter, a diagnostic angiographic catheter, a stent retriever catheter, a clot retriever, a balloon catheter, a catheter to facilitate percutaneous valve repair or replacement, or an ablation catheter; further including a microcontroller electronically coupled with the sensor; further including a second hub configured to adjust an axial position of a second interventional device, a second driven magnet coupled to the second hub, a second hub adapter configured to move in the at least one direction based on an input provided by a user of the robotic control system, a second drive magnet coupled with the second hub adapter and configured to couple with the second driven magnet such that the second driven magnet moves in response to movement of the second drive magnet, a second sensor coupled with the second hub or the second hub adapter and configured to measure a magnitude of a second magnetic field; and/or wherein the hub adapter is coupled with a drive belt.

Also disclosed herein are embodiments of a method of controlling a movement of an interventional device through a sterile barrier, including magnetically coupling a hub on a sterile side of the sterile barrier with a hub adapter on a non-sterile side of the sterile barrier such that the hub moves in response to movement of the hub adapter, moving the hub in at least one direction by moving the hub adapter in the at least one direction while the hub is biased to remain in alignment with the hub adapter in the at least one direction by the magnetic coupling, and determining a distance between the hub and the hub adapter and/or an external force applied to the hub when the hub is offset in the at least one direction from the hub adapter based on measurement of a magnetic field from the magnetic coupling on a sensor coupled to the hub or the hub adapter.

Any embodiments of the methods, devices and systems disclosed herein can include, in additional embodiments, one or more of the following steps, features, components, and/or details, in any combination with any of the other steps, features, components, and/or details of any other embodiments disclosed herein: wherein the first interventional device is a guide catheter, a procedure catheter, an access catheter, or a guidewire; wherein determining the distance between the hub and the hub adapter when the hub is offset in the at least one direction with the hub adapter comprises measuring a magnitude in the at least one direction of the magnetic field on the sensor; further including determining a magnitude of an external force acting on the hub in the at least one direction when the hub is offset in the at least one direction from the hub adapter; wherein the sterile barrier does not include any discontinuities between the hub and the hub adapter; including magnetically coupling a second hub on the sterile side of the sterile barrier with a second hub adapter on the non-sterile side of the sterile barrier such that the second hub moves in response to movement of the second hub adapter; including moving the second hub in at least one direction by moving the second hub adapter in the at least one direction while the second hub is biased to remain in alignment with the second hub adapter in the at least one direction by the magnetic coupling between the second hub adapter and the second hub; including determining a second distance between the second hub and the second hub adapter and/or a second external force applied to the second hub when the second hub is offset in the at least one direction from the second hub adapter based on measurement of a magnetic field from the magnetic coupling on a sensor coupled to the second hub or the second hub adapter; including comparing the distance between the hub and the hub adapter and the second distance between the second hub and the second hub adapter; including comparing a magnitude and/or a direction of the second external force applied to the second hub with a magnitude and/or a direction of the external force applied to the hub; and/or including determining a condition of the hub and/or the second hub based on the comparison of the magnitude and/or the direction of the second external force applied to the second hub with the magnitude and/or the direction of the external force applied to the hub.

Some embodiments disclosed herein are directed to a robotic control system. In any embodiments disclosed herein, the f

Also disclosed herein are embodiments of a robotic control system that include a hub configured to adjust an axial position of an interventional device, a driven magnet coupled with the hub, a hub adapter configured to move in at least one direction based on an input provided by a user of the robotic control system, a drive magnet coupled with the hub adapter and configured to magnetically couple with the driven magnet in an operable state of the robotic control system such that the driven magnet moves in response to a movement of the drive magnet, and a sensor coupled with the hub or the hub adapter and configured to measure a magnitude of a magnetic field on the sensor.

In any embodiments of the robotic control system or methods of using the robotic control system disclosed herein can include a second hub configured to adjust an axial position of a second interventional device, a second driven magnet coupled to the second hub, a second hub adapter configured to move in the at least one direction based on an input provided by a user of the robotic control system, a second drive magnet coupled with the second hub adapter and configured to couple with the second driven magnet such that the second driven magnet moves in response to movement of the second drive magnet, and a second sensor coupled with the second hub or the second hub adapter and configured to measure a magnitude of a second magnetic field.

In any embodiments of the robotic control system or methods of using the robotic control system disclosed herein, the hub adapter can be configured to move proximally or distally along a track in response to the input provided by a user of the robotic control system and/or based on commands automatically generated by a controller of the robotic control system. In some embodiments, the track can be a linear straight gear rack and each hub adapter can have a motor having a pinion gear configured to move along the linear straight gear rack. The first hub adapter can be configured to move in the at least one axial direction on a rack and pinion linear actuator in response to an input provided by a user of the robotic control system. In some embodiments, any of the hub adapters can be coupled with a drive belt and can be configured to move in an axial direction using the drive belt.

Any embodiments of the devices, systems, and/or methods disclosed herein can include, in additional embodiments, one or more of the following steps, features, components, and/or details, in any combination with any of the other features, components, details, and/or steps of any other embodiments disclosed herein: wherein the drive magnet and driven magnet magnetically couple the hub adapter to the hub when the hub is within a predetermined distance of the hub adapter in the at least one direction; wherein the driven magnet biases the hub to remain in approximate alignment with the hub adapter in the at least one direction when the hub adapter moves in the at least one direction; wherein the sensor is a magnetometer; wherein the sensor is an inductance sensor; wherein the robotic control system is configured to determine a relative distance in the at least one direction between the hub adapter and the hub when the hub is at least partially offset from the hub adapter in the at least one direction based on the measured magnitude of the magnetic field on the sensor; wherein the robotic control system is configured to provide a warning when the relative distance reaches or exceeds a threshold value; wherein the robotic control system is configured to increase an intensity of the warning when the relative distance increases; wherein the robotic control system is configured to increase an intensity of the warning by increasing a size of a warning symbol displayed by the robotic control system, by increasing a color tone or an opacity of a warning symbol, by changing a color of the warning symbol, and/or by increasing a volume level or changing a pitch of an audible warning; wherein the robotic control system is configured to determine a magnitude of a net external force acting on the hub in the at least one direction based on the measured magnitude of the magnetic field on the sensor; wherein the robotic control system is configured to provide an alert when the net external force in the at least one direction reaches or exceeds a threshold value; wherein the robotic control system is configured to automatically implement a correction action when the net external force in the at least one direction reaches or exceeds a threshold value; wherein the correction action comprises stopping any movement of the hub or the hub adapter, stopping any movement of the hub or the hub adapter in any direction which would increase the net external force, unloading one or more catheters or other devices to reduce the net external force on the hub, providing information to a user of the system to assist the user in reducing the net external force on the hub, and/or providing specific instructions to the user to instruct the user on maneuvers that would reduce the net external force on the hub; wherein the correction action comprises moving the hub adapter in a direction that reduces the net external force acting on the hub; wherein the robotic control system is configured to determine a relative distance in the at least one direction between the hub adapter and the hub when the hub is at least partially offset from the hub adapter in the at least one direction based on the measured magnitude of the magnetic field on the sensor, and/or wherein the robotic control system is configured to determine a magnitude of a net external force acting on the hub in the at least one direction based at least in part on the relative distance in the at least one direction between the hub adapter and the hub when the hub is offset from the hub adapter in the at least one direction; wherein the hub is coupled to the hub adapter across a sterile barrier; wherein the hub is positioned on a sterile side of the sterile barrier and the hub adapter is positioned on a nonsterile side of the sterile barrier; wherein the sensor is coupled with the hub adapter; wherein the interventional device is a guide catheter, a procedure catheter, an access catheter, or a guidewire; and/or wherein the interventional device is an aspiration catheter, an embolic deployment catheter, a stent deployment catheter, a flow diverter deployment catheter, a diagnostic angiographic catheter, a stent retriever catheter, a clot retriever, a balloon catheter, a catheter to facilitate percutaneous valve repair or replacement, or an ablation catheter.

Any embodiments of the devices, systems, and/or methods disclosed herein can include, in additional embodiments, one or more of the following steps, features, components, and/or details, in any combination with any of the other features, components, details, and/or steps of any other embodiments disclosed herein: wherein the robotic control system or method of using the robotic control system further includes a microcontroller electronically coupled with the sensor; wherein the robotic control system or method of using the robotic control system further includes a data processor configured to receive data from the sensor and determine a relative displacement of the hub relative to the hub adapter and/or a net eternal force acting on the hub based on the data from the sensor; wherein the system is configured to determine a relative displacement of the hub relative to the hub adapter and a direction of the displacement of the hub relative to the hub adapter; wherein the system is configured to determine whether the hub has contacted and is pushing against another adjacent hub, for example and without limitation, whether a hub has contacted and is pushing against another hub and/or whether a hub is magnetically tethered to a hub adapter may be determined based on a determined displacement between the hub and the hub adapter and/or a determined force acting on the hub; wherein the sensor is used to determine if the hub is correctly positioned and oriented relative to the hub adapter during an initial setup procedure of the robotic control system; wherein the robotic control system or method of using the robotic control system further includes at least three hub adapters each having a magnet and three corresponding hubs, wherein each of the three hub adapters has a sensor configured to measure a magnitude of a magnetic field on the sensor from the magnet of each of the corresponding hubs; wherein the robotic control system or method of using the robotic control system further includes at least four hub adapters each having a magnet and four corresponding hubs, wherein each of the four hub adapters has a sensor configured to measure a magnitude of a magnetic field on the sensor from the magnet of each of the corresponding hubs; wherein the robotic control system or method of using the robotic control system further includes a controller configured to execute control functions to at least cause a movement of the hub adapter based on force patterns derived from data of the magnitude of the magnetic field generated by the sensor; wherein the robotic control system or method of using the robotic control system further includes a controller configured to execute control functions to at least cause a movement of the hub adapter to reduce a net force acting on the hub based on force patterns derived from data of the magnitude of the magnetic field generated by the sensor; wherein the robotic control system is configured to determine that a range of motion of an anti-buckling component of the robotic control system is approaching an out-of-range position or that a range of motion of the anti-buckling component is being exceeded; wherein the hub adapter is further configured to move in at least one direction based on a command automatically generated by a controller of the robotic control system; wherein the robotic control system or method of using the robotic control system further includes an additional magnet coupled with the hub, wherein the additional magnet is configured to produce the magnetic field, and wherein the sensor is coupled with the hub adapter and is configured to measure the magnitude of the magnetic field that is produced by the additional magnet; and/or wherein the driven magnet is a ring magnet having an opening axially through a center thereof and the drive magnet is a ring magnet having an opening axially through a center thereof.

Also disclosed herein are embodiments of a robotic control system that can include a hub configured to adjust an axial position of an interventional device, a hub adapter configured to move distally or proximally in an axial direction based at least on an input provided by a user of the robotic control system, one or more magnets coupled with at least the hub, and a sensor coupled with the hub or the hub adapter and configured to measure a magnitude and a direction of a magnetic field of one of the one or more magnets so that the robotic control system can determine a magnitude and a direction of a displacement of the hub relative to the hub adapter. In some embodiments, the robotic control system or method of using the robotic control system can further include a drive magnet coupled with the hub adapter and a driven magnet coupled with the hub, wherein the drive magnet is configured to magnetically couple with the driven magnet in an operable state of the robotic control system such that the hub and the driven magnet move distally or proximally in the axial direction in response to a movement of the hub adapter and the drive magnet. In any embodiments disclosed herein, any of the magnets, including any of the magnets that are positioned and configured to be sensed by a sensor, can be a ring magnet.

Any embodiments of the devices, systems, and/or methods disclosed herein can include, in additional embodiments, one or more of the following steps, features, components, and/or details, in any combination with any of the other features, components, details, and/or steps of any other embodiments disclosed herein: wherein the robotic control system further includes a drive magnet coupled with the hub adapter and a driven magnet coupled with the hub, wherein the drive magnet is configured to magnetically couple with the driven magnet in an operable state of the robotic control system such that the hub and the driven magnet move distally or proximally in the axial direction in response to a movement of the hub adapter and the drive magnet; wherein the driven magnet biases the hub to remain in approximate alignment with the hub adapter in the axial direction when the hub adapter moves in the axial direction; wherein the one of the one or more magnets is a ring magnet; wherein the sensor is a magnetometer; wherein the sensor is an inductance sensor; wherein the robotic control system is configured to provide a warning when the magnitude of displacement reaches or exceeds a threshold value; wherein the robotic control system is configured to increase an intensity of the warning when the magnitude of displacement increases; wherein the robotic control system is configured to increase an intensity of the warning by increasing a size of a warning symbol displayed by the robotic control system, by increasing a color tone or an opacity of a warning symbol, by changing a color of the warning symbol, and/or by increasing a volume level or changing a pitch of an audible warning; wherein the robotic control system is configured to determine a magnitude of a net external force acting on the hub in at least one direction based on the measured magnitude of the magnetic field on the sensor; wherein the robotic control system is configured to provide an alert when the net external force in the at least one direction reaches or exceeds a threshold value; wherein the robotic control system is configured to automatically implement a correction action when the net external force in the at least one direction reaches or exceeds a threshold value; wherein the correction action comprises stopping any movement of the hub or the hub adapter, stopping any movement of the hub or the hub adapter in any direction which would increase the net external force, unloading one or more catheters or other devices to reduce the net external force on the hub, providing information to a user of the system to assist the user in reducing the net external force on the hub, and/or providing specific instructions to the user to instruct the user on maneuvers that would reduce the net external force on the hub; wherein the correction action comprises moving the hub adapter in a direction that reduces the net external force acting on the hub; wherein the hub is coupled to the hub adapter across a sterile barrier; and/or wherein the hub is positioned on a sterile side of the sterile barrier and the hub adapter is positioned on a nonsterile side of the sterile barrier; wherein the sensor is coupled with the hub adapter.

Any embodiments of the devices, systems, and/or methods disclosed herein can include, in additional embodiments, one or more of the following steps, features, components, and/or details, in any combination with any of the other features, components, details, and/or steps of any other embodiments disclosed herein: wherein the interventional device is a guide catheter, a procedure catheter, an access catheter, or a guidewire; wherein the interventional device is an aspiration catheter, an embolic deployment catheter, a stent deployment catheter, a flow diverter deployment catheter, a diagnostic angiographic catheter, a stent retriever catheter, a clot retriever, a balloon catheter, a catheter to facilitate percutaneous valve repair or replacement, or an ablation catheter; wherein the robotic control system further includes a microcontroller electronically coupled with the sensor; wherein the hub adapter is configured to move proximally or distally along a track in response to the input provided by a user of the robotic control system and/or based on commands automatically generated by a controller of the robotic control system; wherein the track is a linear straight gear rack and each hub adapter has a motor having a pinion gear configured to move along the linear straight gear rack; wherein the hub adapter is configured to move in the axial direction on a rack and pinion linear actuator in response to an input provided by a user of the robotic control system; wherein the robotic control system further includes a data processor configured to receive data from the sensor and determine a relative displacement of the hub relative to the hub adapter and/or a net eternal force acting on the hub based on the data from the sensor; wherein the system is configured to determine whether the hub has contacted and is pushing against another adjacent hub; For example, whether a hub has contacted and is pushing against another hub and/or whether a hub is magnetically tethered to a hub adapter may be determined based on a determined displacement between the hub and the hub adapter and/or a determined force acting on the hub; wherein the sensor is used to determine if the hub is correctly positioned and oriented relative to the hub adapter during an initial setup procedure of the robotic control system; wherein the robotic control system includes at least three hub adapters each having a magnet and three corresponding hubs, wherein each of the three hub adapters has a sensor configured to measure a magnitude of a magnetic field on the sensor from the magnet of each of the corresponding hubs; wherein the robotic control system includes at least four hub adapters each having a magnet and four corresponding hubs, wherein each of the four hub adapters has a sensor configured to measure a magnitude of a magnetic field on the sensor from the magnet of each of the corresponding hubs; wherein the robotic control system includes a controller configured to execute control functions to at least cause a movement of the hub adapter based on force patterns derived from data of the magnitude of the magnetic field generated by the sensor; wherein the robotic control system includes a controller configured to execute control functions to at least cause a movement of the hub adapter to reduce a net force acting on the hub based on force patterns derived from data of the magnitude of the magnetic field generated by the sensor; wherein the robotic control system is configured to determine that a range of motion of an anti-buckling component of the robotic control system is approaching an out-of-range position or that a range of motion of the anti-buckling component is being exceeded; and/or wherein the hub adapter is further configured to move in at least one direction based on a command automatically generated by a controller of the robotic control system.

In any embodiments disclosed herein, the robotic control system can further include a second hub configured to adjust an axial position of a second interventional device, a second driven magnet coupled to the second hub, a second hub adapter configured to move in the at least one direction based on an input provided by a user of the robotic control system, a second drive magnet coupled with the second hub adapter and configured to couple with the second driven magnet such that the second driven magnet moves in response to movement of the second drive magnet, and a second sensor coupled with the second hub or the second hub adapter and configured to measure a magnitude of a second magnetic field.

Also disclosed herein are embodiments of a robotic control system that includes a hub configured to adjust an axial position of an interventional device, a driven magnet coupled with the hub, a hub adapter configured to move distally or proximally in an axial direction based at least on an input provided by a user of the robotic control system, a drive magnet coupled with the hub adapter and configured to couple with the driven magnet coupled with the hub such that the driven magnet moves in response to a movement of the drive magnet, a sensor coupled with the hub adapter configured to measure a magnitude of a magnetic field from a magnet coupled with the hub, and a controller configured to determine a magnitude of a net external force acting on the hub in the axial direction based on the measured magnitude of the magnetic field on the sensor. In some embodiments, the robotic control system can be configured to output a warning to the user of the robotic control system when the magnitude of the net external force acting on the hub in the axial direction reaches a threshold value that is a predetermined percentage of a breakaway force.

Any embodiments of the devices, systems, and/or methods disclosed herein can include, in additional embodiments, one or more of the following steps, features, components, and/or details, in any combination with any of the other features, components, details, and/or steps of any other embodiments disclosed herein: wherein the drive magnet and driven magnet magnetically couple the hub adapter to the hub when the hub is within a predetermined distance of the hub adapter in the axial direction; wherein the driven magnet biases the hub to remain in approximate alignment with the hub adapter in the axial direction when the hub adapter moves in the axial direction; wherein the sensor is a magnetometer; wherein the robotic control system is configured to increase an intensity of the warning when the magnitude of the net external force acting on the hub in the axial direction increases; wherein the robotic control system is configured to increase an intensity of the warning by increasing a size of a warning symbol displayed by the robotic control system, by increasing a color tone or an opacity of a warning symbol, by changing a color of the warning symbol, and/or by increasing a volume level or changing a pitch of an audible warning; wherein the robotic control system is configured to automatically implement a correction action when the magnitude of the net external force acting on the hub in the axial direction reaches or exceeds the threshold value; wherein the correction action comprises stopping any movement of the hub or the hub adapter, stopping any movement of the hub or the hub adapter in any direction which would increase the net external force, unloading one or more catheters or other devices to reduce the net external force on the hub, providing information to a user of the system to assist the user in reducing the net external force on the hub, and/or providing specific instructions to the user to instruct the user on maneuvers that would reduce the net external force on the hub; wherein the correction action comprises moving the hub adapter in a direction that reduces the net external force acting on the hub; wherein the robotic control system is configured to impede the movement of the hub adapter in a direction that would increase the net external force acting on a hub if the net external force acting on the hub reaches the threshold value; wherein the threshold value is at least 70% of a breakaway force between the hub and the hub adapter; wherein the threshold value is at least 80% of a breakaway force between the hub and the hub adapter; wherein the hub is coupled to the hub adapter across a sterile barrier; and/or wherein the hub is positioned on a sterile side of the sterile barrier and the hub adapter is positioned on a nonsterile side of the sterile barrier.

In any embodiments disclosed herein, the robotic control system or method of using the robotic control system can further include a second hub configured to adjust an axial position of a second interventional device, a second driven magnet coupled to the second hub, a second hub adapter configured to move in the axial direction based on an input provided by a user of the robotic control system, a second drive magnet coupled with the second hub adapter and configured to couple with the second driven magnet such that the second driven magnet moves in response to movement of the second drive magnet, and a second sensor coupled with the second hub or the second hub adapter and configured to measure a magnitude of a second magnetic field.

Any embodiments of the devices, systems, and/or methods disclosed herein can include, in additional embodiments, one or more of the following steps, features, components, and/or details, in any combination with any of the other features, components, details, and/or steps of any other embodiments disclosed herein: wherein the hub adapter is configured to move proximally or distally along a track in response to the input provided by a user of the robotic control system and/or based on commands automatically generated by a controller of the robotic control system: wherein the track is a linear straight gear rack and each hub adapter has a motor having a pinion gear configured to move along the linear straight gear rack; wherein the system is configured to determine whether the hub has contacted and is pushing against another adjacent hub For example, whether a hub has contacted and is pushing against another hub and/or whether a hub is magnetically tethered to a hub adapter may be determined based on a determined displacement between the hub and the hub adapter and/or a determined force acting on the hub; wherein the robotic control system or method of using the robotic control system further include at least three hub adapters each having a magnet and three corresponding hubs, wherein each of the three hub adapters has a sensor configured to measure a magnitude of a magnetic field on the sensor from the magnet of each of the corresponding hubs; wherein the robotic control system or method of using the robotic control system further includes a controller configured to execute control functions to at least cause a movement of the hub adapter to reduce a net force acting on the hub based on force patterns derived from data of the magnitude of the magnetic field generated by the sensor; and/or wherein the driven magnet is a ring magnet having an opening axially through a center thereof and the drive magnet is a ring magnet having an opening axially through a center thereof.

Also disclosed herein are embodiments of a method of controlling a movement of an interventional device through a sterile barrier. In some embodiments, the method of controlling a movement of an interventional device through the sterile barrier can include magnetically coupling a hub on a sterile side of the sterile barrier with a hub adapter on a non-sterile side of the sterile barrier such that the hub moves in response to a movement of the hub adapter, moving the hub in at least one direction by moving the hub adapter in the at least one direction while the hub is biased to remain in alignment with the hub adapter in the at least one direction by the magnetic coupling, and determining a distance between the hub and the hub adapter and/or a net external force applied to the hub when the hub is offset in the at least one direction from the hub adapter based on measurement of a magnetic field from the magnetic coupling on a sensor coupled to the hub or the hub adapter.

Any embodiments of the methods, devices and systems disclosed herein can include, in additional embodiments, one or more of the following steps, features, components, and/or details, in any combination with any of the other steps, features, components, and/or details of any other embodiments disclosed herein: wherein the first interventional device is a guide catheter, a procedure catheter, an access catheter, or a guidewire; wherein determining the distance between the hub and the hub adapter when the hub is offset in the at least one direction with the hub adapter comprises measuring a magnitude in the at least one direction of the magnetic field on the sensor; wherein the robotic control system or method of using the robotic control system further includes determining a magnitude of an external force acting on the hub in the at least one direction when the hub is offset in the at least one direction from the hub adapter; wherein the sterile barrier does not include any discontinuities between the hub and the hub adapter; including comparing the distance between the hub and the hub adapter and the second distance between the second hub and the second hub adapter; wherein the robotic control system or method of using the robotic control system further includes comparing a magnitude and/or a direction of the second net external force applied to the second hub with a magnitude and/or a direction of the net external force applied to the hub; wherein the robotic control system or method of using the robotic control system further includes determining a condition of the hub and/or the second hub based on the comparison of the magnitude and/or the direction of the second net external force applied to the second hub with the magnitude and/or the direction of the net external force applied to the hub; and/or wherein the robotic control system or method of using the robotic control system further includes moving the hub adapter in the at least one direction along a linear rack gear.

Any embodiments of the method of controlling a movement of an interventional device through a sterile barrier disclosed herein can further include magnetically coupling a second hub on the sterile side of the sterile barrier with a second hub adapter on the non-sterile side of the sterile barrier such that the second hub moves in response to movement of the second hub adapter, moving the second hub in at least one direction by moving the second hub adapter in the at least one direction while the second hub is biased to remain in alignment with the second hub adapter in the at least one direction by the magnetic coupling between the second hub adapter and the second hub, and/or determining a second distance between the second hub and the second hub adapter and/or a second net external force applied to the second hub when the second hub is offset in the at least one direction from the second hub adapter based on measurement of a magnetic field from the magnetic coupling on a sensor coupled to the second hub or the second hub adapter.

Also disclosed herein are embodiments of a method of controlling a movement of an interventional device through a sterile barrier that can include magnetically coupling a hub on a sterile side of the sterile barrier with a hub adapter on a non-sterile side of the sterile barrier such that the hub moves in response to a movement of the hub adapter, moving the hub in at least one direction by moving the hub adapter in the at least one direction along a linear rack gear while the hub is biased to remain in alignment with the hub adapter in the at least one direction by the magnetic coupling, and determining a distance between the hub and the hub adapter and/or an external force applied to the hub when the hub is offset in the at least one direction from the hub adapter based on measurement of a magnetic field from the magnetic coupling on a sensor coupled to the hub or the hub adapter.

Any embodiments of the methods, devices and systems disclosed herein can include, in additional embodiments, one or more of the following steps, features, components, and/or details, in any combination with any of the other steps, features, components, and/or details of any other embodiments disclosed herein: wherein the robotic control system or method of using the robotic control system further includes outputting a warning when a magnitude of the net external force acting on the hub in the axial direction reaches a threshold value including a predetermined percentage of the breakaway force; wherein the threshold value is at least 70% of a breakaway force between the hub and the hub adapter; wherein the threshold value is at least 80% of a breakaway force between the hub and the hub adapter; wherein the robotic control system or method of using the robotic control system further includes impeding the movement of the hub adapter in a direction that would increase the net external force acting on a hub when the net external force acting on the hub reaches a threshold value; wherein the robotic control system or method of using the robotic control system further includes automatically implementing a correction action when the net external force in the at least one direction reaches or exceeds a threshold value; wherein the correction action comprises stopping any movement of the hub or the hub adapter, stopping any movement of the hub or the hub adapter in any direction which would increase the net external force, unloading one or more catheters or other devices to reduce the net external force on the hub, providing information to a user of the system to assist the user in reducing the net external force on the hub, and/or providing specific instructions to the user to instruct the user on maneuvers that would reduce the net external force on the hub; wherein the correction action comprises moving the hub adapter in a direction that reduces the net external force acting on the hub; wherein an interventional device is coupled with the hub, the interventional device being a guide catheter, a procedure catheter, an access catheter, or a guidewire; and/or wherein an interventional device is coupled with the hub, the interventional device being an aspiration catheter, an embolic deployment catheter, a stent deployment catheter, a flow diverter deployment catheter, a diagnostic angiographic catheter, a stent retriever catheter, a clot retriever, a balloon catheter, a catheter to facilitate percutaneous valve repair or replacement, or an ablation catheter.

Also disclosed herein are embodiments of a method of controlling a movement of an interventional device through a sterile barrier that can include magnetically coupling a hub on a sterile side of the sterile barrier with a hub adapter on a non-sterile side of the sterile barrier such that, in an operable state, the hub moves in response to a movement of the hub adapter, moving the hub in at least one direction by moving the hub adapter in the at least one direction while the hub is magnetically coupled with the hub adapter, measuring a magnitude of a magnetic field from a magnet coupled with the hub using a magnetic field sensor coupled with the hub adapter, determining a magnitude of a net external force acting on the hub from the magnitude of the magnetic field from the magnet coupled with the hub, and outputting a warning to the user of the robotic control system when the magnitude of the net external force acting on the hub in the at least one direction reaches a threshold value including a predetermined percentage of the breakaway force. In some embodiments, the method of controlling a movement of an interventional device through a sterile barrier can further include impeding the movement of the hub adapter in a direction that would increase the net external force acting on a hub when the net external force acting on the hub reaches the threshold value. In some embodiments, the method of controlling a movement of an interventional device through a sterile barrier can further include outputting a warning to the user of the robotic control system when the magnitude of the net external force acting on the hub in the at least one direction reaches the threshold value.

In any embodiments disclosed herein, the threshold value can be 70% or at least 70% of a breakaway force between the hub and the hub adapter. In any embodiments disclosed herein, the threshold value can be 80% or at least 80% of a breakaway force between the hub and the hub adapter, or 85% or at least 85%, or 90% or at least 90% of a breakaway force between the hub and the hub adapter, or any values in any of the foregoing ranges.

Also disclosed herein are embodiments of a method of performing a neurovascular procedure. In some embodiments, the embodiments of the method of performing a neurovascular procedure can include controlling the movement of the interventional device through the sterile barrier as described in any embodiments of the method of controlling a movement of an interventional device through a sterile barrier disclosed herein wherein the hub is an access catheter hub that has an access catheter. In some embodiments, the method of performing the neurovascular procedure can further include coupling the access catheter hub to the hub adapter, the hub adapter being movably carried by a support table, driving the access catheter in response to a movement of the hub adapter along the support table until the access catheter is positioned to achieve supra-aortic vessel access, removing the access catheter and the access catheter hub from the hub adapter, and coupling a procedure catheter hub having a procedure catheter to the hub adapter.

### BRIEF DESCRIPTION OF THE DRAWINGS

FIG. 1 is a schematic perspective view of an interventional setup having an imaging system, a patient support table, and a robotic drive system in accordance with the present disclosure.

FIG. 2 is a longitudinal cross section showing the concentric relationship between a guidewire having two degrees of freedom, an access catheter having 3 degrees of freedom and a guide catheter having one degree of freedom.

FIG. 3A is an exploded schematic view of interventional device hubs separated from a support table by a sterile barrier.

FIGS. 3B-3F show an alternate sterile barrier in the form of a shipping tray having one or more storage channels for carrying interventional devices.

FIGS. 3G-3K show embodiments of an alternate sterile barrier having a convex drive surface.

FIGS. 3L and 3M depict an example of a hub that may be used with the sterile barriers of FIGS. 3G-3K.

FIG. 4 is a schematic elevational cross section through a hub adapter having a drive magnet separated from an interventional device hub and driven magnet by a sterile barrier.

FIGS. 5A and 5B schematically illustrate a three interventional device and

a four interventional device assembly.

FIG. 6 is a perspective view of a support table.

FIG. 7 is a close-up view of the motor drive end of a support table.

FIG. 8 is an elevational cross section through a motor and belt drive assembly.

FIG. 9 is a close-up view of a pulley end of the support table.

FIG. 10 is an elevational cross section through a belt pully.

FIG. 11 is a side elevational cross-section through a distal portion of a catheter such as any of those shown in FIGS. 5A and 5B.

FIGS. 12A and 12B schematically illustrate a force sensor integrated into the sidewall of the catheter.

FIGS. 13A and 13B schematically illustrate a sensor for measuring elastic forces at the magnetic coupling between the hub and corresponding hub adapter.

FIG. 14 schematically illustrates a dual encoder torque sensor for use with a catheter of the present disclosure.

FIG. 15 illustrates a clot capture and visualization device that can be integrated into a hub and/or connected to an aspiration line.

FIGS. 16A-16C illustrate an example control mechanism for manipulating interventional devices driven by respective hubs.

FIG. 17 illustrates a side elevational schematic view of an interventional device assembly for supra-aortic access and neuro-interventional procedures.

FIGS. 18A-18E depict an example sequence of steps of introducing a catheter assembly configured to achieve supra-aortic access and neurovascular site access.

FIG. 19 schematically illustrates an embodiment of a mechanical coupling between a drive mechanism and a driven mechanism.

FIGS. 20A-20C depict an example sequence of steps of priming a catheter assembly in a stacked configuration.

FIGS. 21A-21B depict an example sequence of steps of priming a catheter assembly in a stacked configuration.

FIG. 22 depicts an example test system for the priming process depicted in FIGS. 21A-21B.

FIG. 23A shows an example of a catheter assembly.

FIG. 23B shows an example of a catheter assembly after a priming procedure.

FIG. 23C shows an example of a catheter assembly after a priming procedure including relative movement between adjacent catheters.

FIGS. 23D-23F illustrate the example catheter assembly of FIGS. 23A-23C.

FIG. 24 is a schematic representation of a hub and a hub adapter having one or magnets to magnetically couple the hub and hub adapter.

FIG. 25 shows a schematic representation of a hypothetical system having a spring positioned between a hub and a hub adapter.

FIG. 26A shows an example embodiment of a portion of a robotic control system having a plurality of magnetically coupled hub adapters and hubs, with interventional devices coupled with one or more of the hubs.

FIG. 26B illustrates an example embodiment of a magnetically coupled hub adapter and hub and also schematically illustrates a range of different forces that can be applied to the hub and the hub adapter.

FIG. 27 illustrates a wiring schematic that can be used with any embodiments of the robotic control system disclosed herein.

FIG. 28 depicts a schematic of a control system.

FIG. 29A shows an embodiment of a magnet that can be used with any embodiments of the robotic control system disclosed herein.

FIG. 29B shows another embodiment of a magnet that can be used with any embodiments of the robotic control system disclosed herein.

FIG. 30A shows a portion of an embodiment of a drive table having one or more rack and pinion actuators that can be used with any embodiments of the robotic control system disclosed herein.

FIG. 30B shows an enlarged view of an embodiment of a rack and pinion actuator of the embodiment of the drive table shown in FIG. 30A that can be used with any embodiments of the robotic control system disclosed herein.

### DETAILED DESCRIPTION

In certain embodiments, a system is provided for advancing a guide catheter from a femoral artery or radial artery access into the ostium of one of the great vessels at the top of the aortic arch, thereby achieving supra-aortic access. A surgeon can then take over and advance interventional devices into the cerebral vasculature via the robotically placed guide catheter.

In some implementations, the system may additionally be configured to robotically gain intra-cranial vascular access and to perform an aspiration thrombectomy or other neuro vascular procedure.

A drive table is positioned over or alongside the patient, and configured to axially advance, retract, and in some cases rotate and/or laterally deflect two or three or more different (e.g., concentrically or side by side oriented) intravascular devices. One or more hubs may act as instrument couplers that can couple to corresponding interventional devices. The hubs may be moveable along a path along the surface of the drive table to advance or retract the interventional device as desired. Each hub may also contain mechanisms to rotate or deflect the device as desired, and is connected to fluid delivery tubes (not shown) of the type conventionally attached to a catheter hub. Each hub can be in electrical communication with an electronic control system, either via hard wired connection, RF wireless connection or a combination of both.

Each hub is independently movable across the surface of a sterile field barrier membrane carried by the drive table. Each hub is releasably magnetically coupled to a unique hub adapter (also called a carriage or a drive carriage) on the table side of the sterile field barrier. The drive system independently moves each hub in a proximal or distal direction across the surface of the barrier, to move the corresponding interventional device proximally or distally within the patient's vasculature.

The hub adapters on the drive table, which magnetically couple with the hubs to provide linear motion actuation, are universal. Functionality of the catheters/guidewire are provided based on what is contained in the hub and the shaft designs. This allows flexibility to configure the system to do a wide range of procedures using a wide variety of interventional devices on the same drive table. Additionally, the interventional devices and methods disclosed herein can be readily adapted for use with any of a wide variety of other drive systems (e.g., any of a wide variety of robotic surgery drive systems).

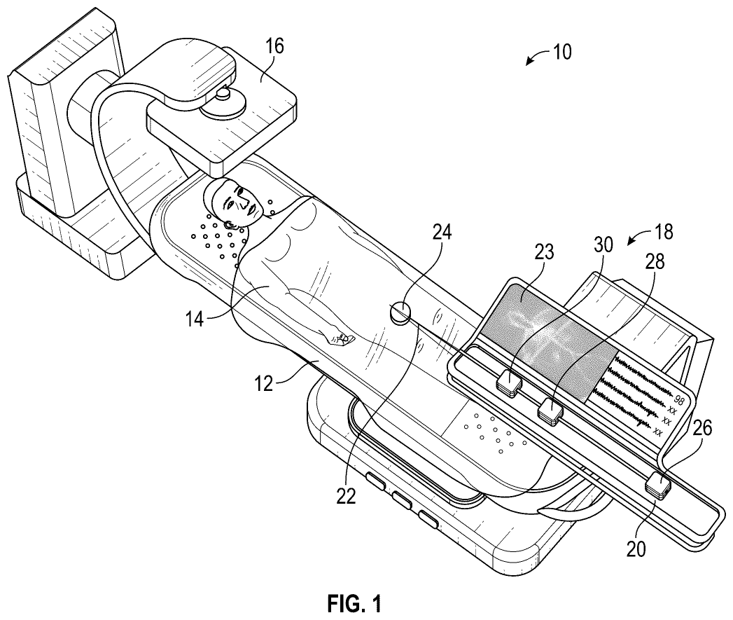

FIG. 1 is a schematic perspective view of an interventional setup 10 having a patient support table 12 for supporting a patient 14. An imaging system 16 may be provided, along with a robotic interventional device drive system 18 in accordance with the present disclosure.

The drive system 18 may include a support table 20 for supporting, for example, a guidewire hub 26, an access catheter hub 28 and a guide catheter hub 30. In the present context, the term ‘access’ catheter can be any catheter having a lumen with at least one distally facing or laterally facing distal opening, that may be utilized to aspirate thrombus, provide access for an additional device to be advanced therethrough or therealong, or to inject saline or contrast media or therapeutic agents.

More or fewer interventional device hubs may be provided depending upon the desired clinical procedure. For example, in certain embodiments, a diagnostic angiogram procedure may be performed using only a guidewire hub 26 and an access catheter hub 28 for driving a guidewire and an access catheter (in the form of a diagnostic angiographic catheter), respectively. Multiple interventional devices 22 extend between the support table 20 and (in the illustrated example) a femoral access point 24 on the patient 14. Depending upon the desired procedure, access may be achieved by percutaneous or cut down access to any of a variety of arteries or veins, such as the femoral artery or radial artery. Although disclosed herein primarily in the context of neuro vascular access and procedures, the robotic drive system and associated interventional devices can readily be configured for use in a wide variety of additional medical interventions, in the peripheral and coronary arterial and venous vasculature, gastrointestinal system, lymphatic system, cerebral spinal fluid lumens or spaces (such as the spinal canal, ventricles, and subarachnoid space), pulmonary airways, treatment sites reached via trans ureteral or urethral or fallopian tube navigation, or other hollow organs or structures in the body (for example, in intra-cardiac or structural heart applications, such as valve repair or replacement, or in any endoluminal procedures).

A display 23 such as for viewing fluoroscopic images, catheter data (e.g., fiber Bragg grating fiber optics sensor data or other force or shape sensing data) or other patient data may be carried by the support table 20 and or patient support 12. Alternatively, the physician input/output interface including display 23 may be remote from the patient, such as behind radiation shielding, in a different room from the patient, or in a different facility than the patient.

In the illustrated example, a guidewire hub 26 is carried by the support table 20 and is moveable along the table to advance a guidewire into and out of the patient 14. An access catheter hub 28 is also carried by the support table 20 and is movable along the table to advance the access catheter into and out of the patient 14. The access catheter hub may also be configured to rotate the access catheter in response to manipulation of a rotation control, and may also be configured to laterally deflect a deflectable portion of the access catheter, in response to manipulation of a deflection control.

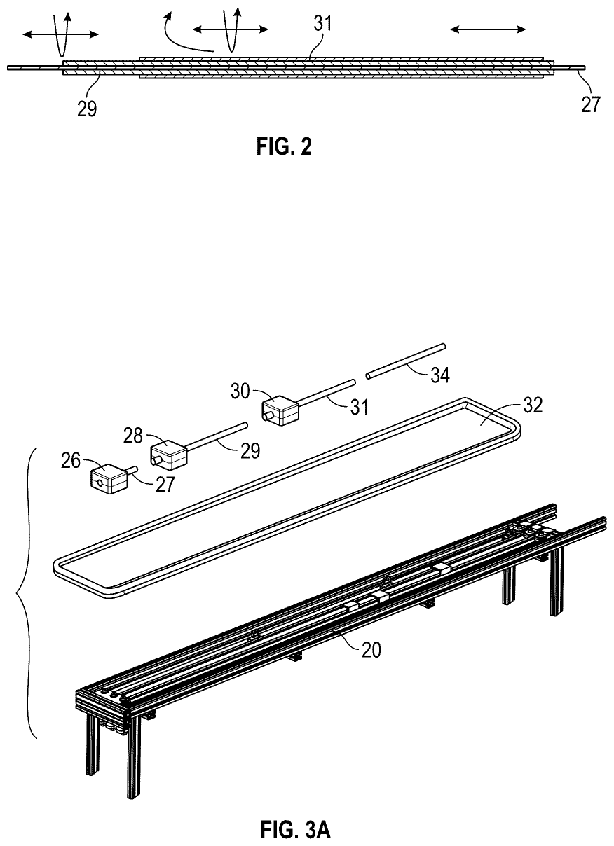

FIG. 2 is a longitudinal cross section schematically showing the motion relationship between a guidewire 27 having two degrees of freedom (axial and rotation), an access catheter 29 having three degrees of freedom (axial, rotational and lateral deflection) and a guide catheter 31, having one degree of freedom (axial).

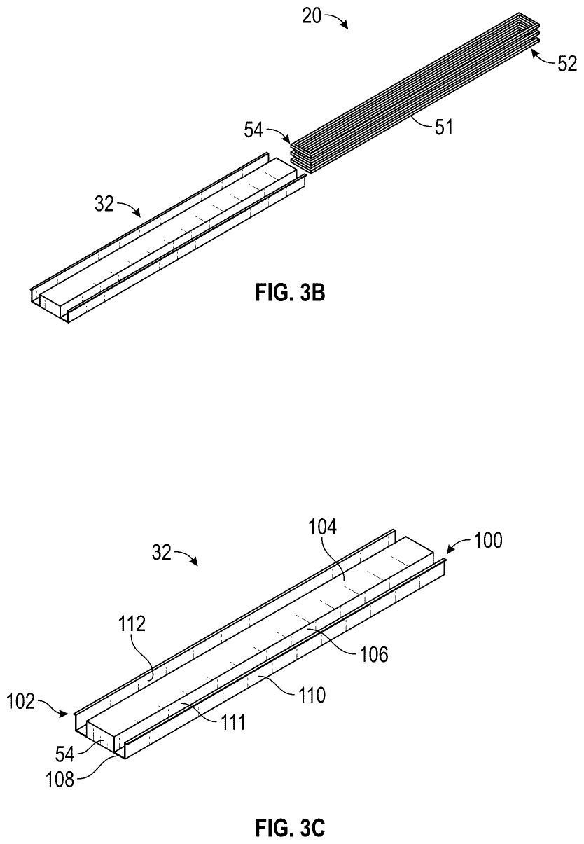

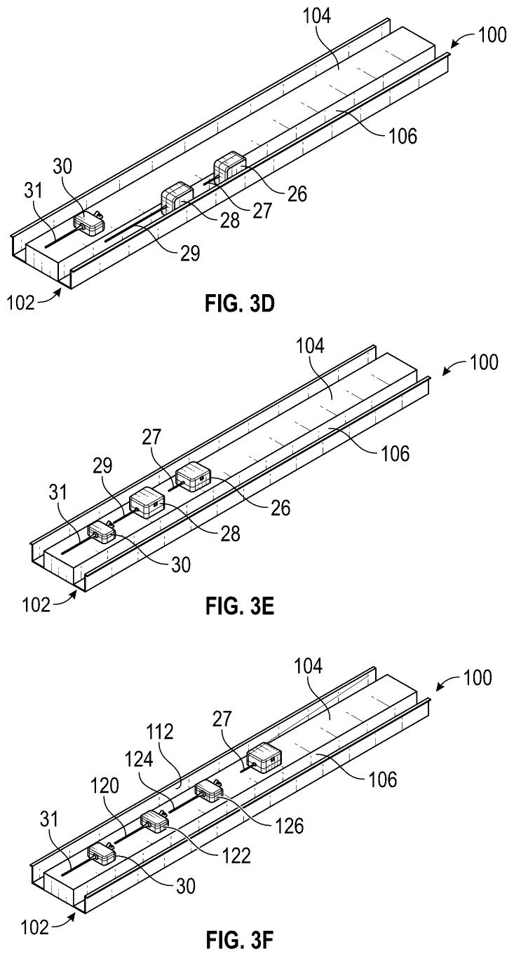

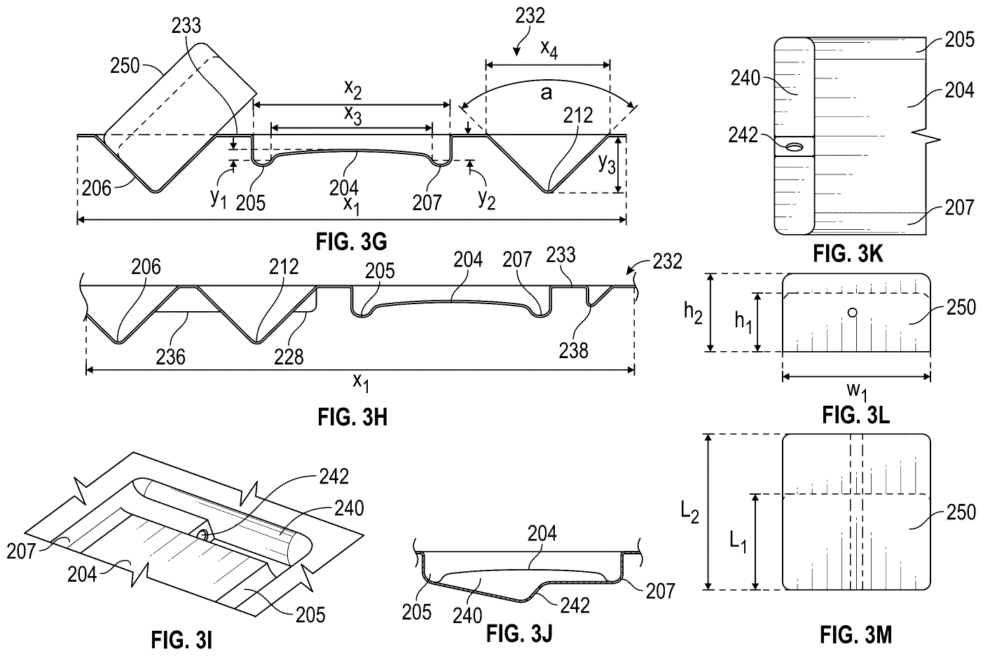

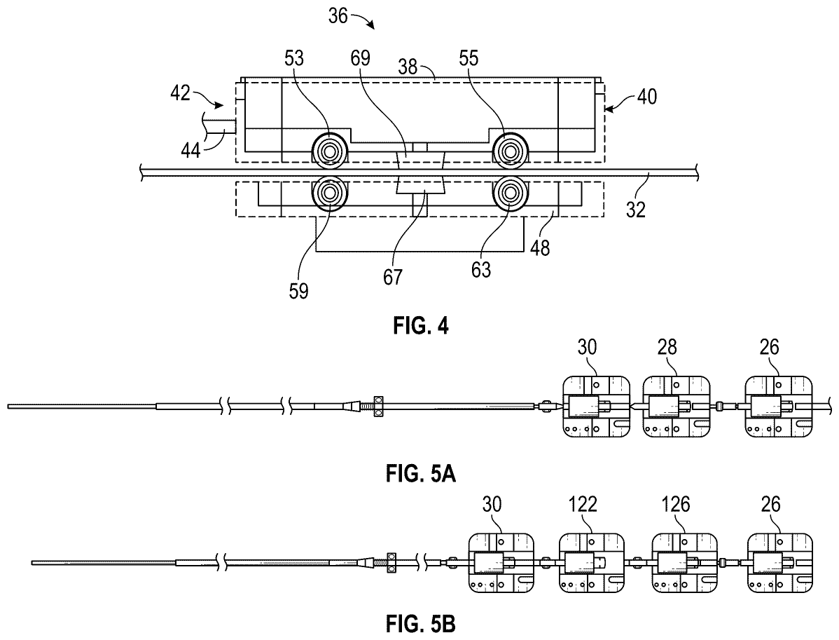

Referring to FIG. 3A, the support table 20 includes a drive mechanism described in greater detail below, to independently drive the guidewire hub 26, access catheter hub 28, and guide catheter hub 30. An anti-buckling feature 34 may be provided in a proximal anti-buckling zone for resisting buckling of the portion of the interventional devices spanning the distance between the support table 20 and the femoral artery access point 24. The anti-buckling feature 34 may comprise a plurality of concentric telescopically axially extendable and collapsible tubes through which the interventional devices extend.

Alternatively, a proximal segment of one or more of the device shafts may be configured with enhanced stiffness to reduce buckling under compression. For example, a proximal reinforced segment may extend distally from the hub through a distance of at least about 5 centimeters or 10 centimeters but typically no more than about 120 centimeters or 100 centimeters to support the device between the hub and the access point 24 on the patient. Reinforcement may be accomplished by using metal or polymer tubing or embedding at least one or two or more axially extending elements into the wall of the device shafts, such as elongate wires or ribbons. In some implementations, the extending element may be hollow and protect from abrasion, buckling, or damage at the inputs and outputs of the hubs. In some embodiments, the hollow extending element may be a hollow and flexible coating attached to a hub. The hollow, extending element (e.g., a hollow and flexible coating) may cover a portion of the device shaft when threaded through the hubs. In some embodiments in which the hollow extending element is a coating, the coating may be attached to a portion of a hub such that threading the catheter device through the hub 26, 28, or 30 threads the catheter device through the coating as well. In some implementations, an anti-buckling device may be installed on or about or surrounding a device shaft to avoid misalignment or insertion angle errors between hubs or between a hub and an insertion point. The anti-buckling device may be a laser cut hypotube, a spring, telescoping tubes, tensioned split tubing, or the like.

In some implementations, a number of deflection sensors may be placed along a catheter length to identify buckling. Identifying buckling may be performed by sensing that a hub is advancing distally, while the distal tip of the catheter or interventional device has not moved. In some implementations, the buckling may be detected by sensing that an energy load (e.g., due to friction) has occurred between catheter shafts.

Alternatively, thin tubular stiffening structures can be embedded within or carried over the outside of the device wall, such as a tubular polymeric extrusion or length of hypo-tube. Alternatively, a removable stiffening mandrel may be placed within a lumen in the proximal segment of the device, and proximally removed following distal advance of the hub towards the patient access site, to prevent buckling of the proximal shafts during distal advance of the hub. Alternatively, a proximal segment of one or more of the device shafts may be constructed as a tubular hypo tube, which may be machined (e.g., with a laser) so that its mechanical properties vary along its length. This proximal segment may be formed of stainless steel, nitinol, and/or cobalt chrome alloys, optionally in combination with polymer components which may provide for lubricity and hydraulic sealing. In some embodiments, this proximal segment may be formed of a polymer, such as polyether ether ketone (PEEK). Alternatively, the wall thickness or diameter of the interventional device can be increased in the anti-buckling zone.

In certain embodiments, a device shaft having advanced stiffness (e.g., axially and torsionally) may provide improved transmission of motion from the proximal end of the device shaft to the distal end of the device shaft. For example, the device shafts may be more responsive to motion applied at the proximal end. Such embodiments may be advantageous for robotic driving in the absence of haptic feedback to a user.

In some embodiments, a flexible coating can be applied to a device shaft and/or hub to reduce frictional forces between the device shaft and/or hub and a second device shaft when the second device shaft passes therethrough.

The interventional device hubs may be separated from the support table 20 by sterile barrier 32. Sterile barrier 32 may comprise a thin plastic membrane such as polyethylene terephthalate (PET), polyethylene terephthalate glycol (PETG), polyethylene terephthalate (PETE), high-density polyethylene (HDPE), polyvinyl chloride (PVC), low-density polyethylene (LDPE), polypropylene (PP), polystyrene (PS), or styrene. This allows the support table 20 and associated drive system to reside on a non-sterile (lower) side of sterile barrier 32. The guidewire hub 26, access catheter hub 28, guide catheter hub 30 and the associated interventional devices are all on a sterile (top) side of the sterile barrier 32. The sterile barrier is preferably waterproof and can also serve as a tray used in the packaging of the interventional devices, discussed further below. The interventional devices can be provided individually or as a coaxially preassembled kit that is shipped and stored in the tray and enclosed within a sterile packaging.

FIGS. 3B-3F schematically illustrate an alternate sterile barrier in the form of a dual function sterile barrier for placement on the support table during the interventional procedure, and shipping tray, having one or more storage channels for carrying sterile interventional devices. The sterile barrier may also act as a sterile work surface for preparation of catheters or other devices during a procedure.

Referring to FIGS. 3B and 3C, there is illustrated a sterile barrier 32 in the form of a pre-shaped tray, for fitting over an elongate support table 20. In use, the elongate support table 20 would be positioned below the sterile barrier 32. The sterile barrier 32 extends between a proximal end 100 and a distal end 102 and includes an upper support surface 104 for supporting the interventional device hubs. In one implementation, the support surface 104 has an axial length greater than the length of the intended interventional devices, in a linear drive configuration.

The length of support surface 104 will typically be at least about 100 centimeters and within the range of from about 100 centimeters to about 2.7 meters. Shorter lengths may be utilized in a system configured to advance the drive couplers along an arcuate path. In some embodiments, two or more support surfaces may be used instead of a single support surface 104. The two or more support surfaces may have a combined length between 100 centimeters to about 2.7 meters. The width of the linear drive table is preferably no more than about 30 to about 80 centimeters.

At least a first channel 106 may be provided, extending axially at least a portion of the length of the support table 20. In the illustrated implementation, first channel 106 extends the entire length of the support table 20. Preferably, the first channel 106 has a sufficient length to hold the interventional devices, and sufficient width and depth to hold the corresponding hubs (for example, by providing lateral support to prevent dislodgment of the hubs when forces are applied to the hubs). First channel 106 is defined within a floor 108, outer side wall 110 and inner side wall 111, forming an upwardly facing concavity. Optionally, a second channel 112 may be provided. Second channel 112 may be located on the same side or the opposite side of the upper support surface 104 from the first channel 106. Two or three or more additional recesses such as additional channels or wells may be provided, to hold additional medical devices or supplies that may be useful during the interventional procedure as well as to collect fluids and function as wash basins for catheters and related devices.

Referring to FIG. 3D, the guide catheter hub 30 is shown positioned on the upper support surface 104, and magnetically coupled to the corresponding coupler holding the drive magnets, positioned beneath the sterile barrier 32. The access catheter hub 28 and access catheter 29, and guidewire hub 26 and guidewire 27 are illustrated residing within the first channel 106 such as before introduction through the guide catheter 31 or following removal from the guide catheter 31.

The interventional devices may be positioned within the channel 106 and enclosed in a sterile barrier for shipping. At the clinical site, an upper panel of the sterile barrier may be removed, or a tubular sterile barrier packaging may be opened and axially removed from the support table 20 and sterile barrier 32 assembly, exposing the sterile top side of the sterile barrier tray and any included interventional devices. The interventional devices may be separately carried in the channel, or preassembled into an access assembly or procedure assembly, discussed in additional detail below.

FIGS. 3D-3F illustrate the support table with sterile barrier in place, and in FIG. 3E, the interventional devices configured in an access assembly for aortic access, following coupling of the access assembly to the corresponding hub adapters beneath the sterile barrier. The access assembly may be preassembled with the guidewire fully advanced through the access catheter which is in turn fully advanced through the guide catheter. In embodiments in which the access catheter or other catheters are pre-shaped (i.e., pre-curved or not straight), the guidewire and/or outer catheters may be positioned so that relatively stiff sections are not superimposed with curved stiffer sections of the pre-shaped catheter, for example, to avoid creep or straightening of the pre-shaped catheter and/or introduction of a curve into an otherwise straight catheter. This access assembly may be lifted out of the channel 106 and positioned on the support surface 104 for coupling to the respective drive magnets and introduction into the patient. The guide catheter hub 30 is the distal most hub. Access catheter hub 28 is positioned proximally of the guide catheter hub, so that the access catheter 29 can extend distally through the guide catheter. The guidewire hub 26 is positioned most proximally, in order to allow the guidewire 27 to advance through the access catheter 29 and guide catheter 31.

A procedure assembly is illustrated in FIG. 3F following introduction of the procedure assembly through the guide catheter 31 that was used to achieve supra-aortic access. In this implementation, guide catheter 31 remains the distal most of the interventional devices. A first procedure catheter 120 and corresponding hub 122 is illustrated extending through the guide catheter 31. An optional second procedure catheter 124 and corresponding hub 126 is illustrated extending through the first procedure catheter 120. The guidewire 27 extends through at least a portion of the second procedure catheter 124 in a rapid exchange version of second procedure catheter 124, or the entire length of second procedure catheter 124 in an over the wire implementation.

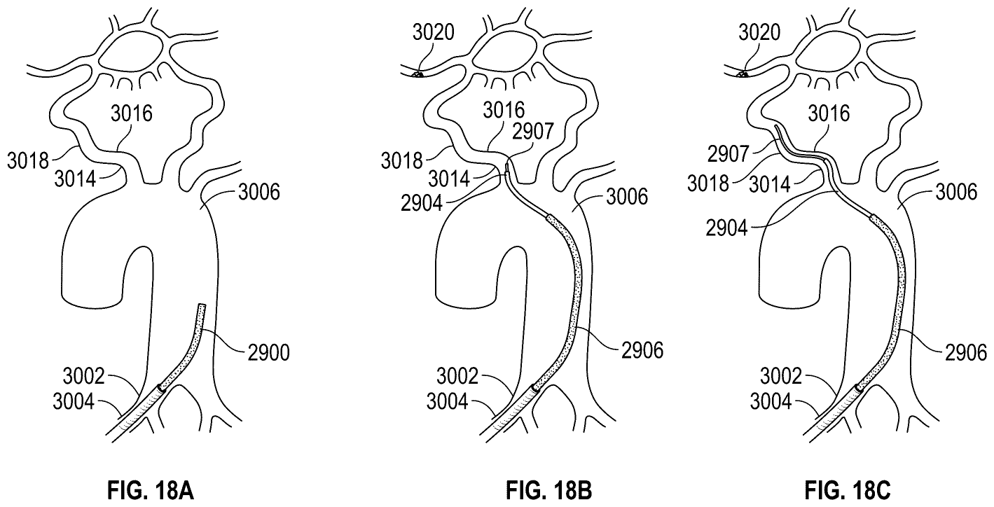

As is discussed in greater detail in connection with FIG. 17, the multi catheter stack may be utilized to achieve both access and the intravascular procedure without the need for catheter exchange. this may be accomplished in either a manual or a robotically driven procedure. In one example, the guide catheter 31 may comprise a catheter having an inner diameter of at least about 0.08 inches and in one implementation about 0.088 inches. The first procedure catheter 120 may comprise a catheter having an inner diameter within the range of from about 0.065 inches to about 0.075 inches and in one implementation catheter 120 has an inner diameter of about 0.071 inches. The second procedure catheter 124 may be an access catheter having an OD sized to permit advance through the first procedure catheter 120. The second procedure catheter maybe steerable, having a deflection control 2908 configured to laterally deflect a distal end of the catheter. The second procedure (access) catheter may also have an inner lumen sized to allow an appropriately sized guidewire to remain inside the second procedure catheter while performing contrast injections through the second procedure catheter.

In certain embodiments, the catheter 31 may be a ‘large bore’ access catheter or guide catheter having a diameter of at least about 0.075 or at least about 0.080 inches in diameter. The catheter 120 may be an aspiration catheter having a diameter within the range of from about 0.060 to about 0.075 inches. The catheter 124 may be a steerable catheter with a deflectable distal tip, having a diameter within the range of from about 0.025 to about 0.050 inches. The guidewire 27 may have a diameter within the range of from about 0.014 to about 0.020 inches. In one example, the catheter 31 may have a diameter of about 0.088 inches, the catheter 120 about 0.071 inches, the catheter 124 about 0.035 inches, and the guidewire 27 may have a diameter of about 0.018 inches.

In one commercial execution, a preassembled access assembly (guide catheter, access catheter and guidewire) may be carried within a first channel on the sterile barrier tray and a preassembled procedure assembly (one or two procedure catheters and a guidewirc) may be carried within the same or a different, second channel on the sterile barrier tray. One or two or more additional catheters or interventional tools may also be provided, depending upon potential needs during the interventional procedure.

FIGS. 3G-3K illustrate embodiments of an alternate sterile barrier having a convex drive surface (e.g., a convex, crowned road like drive surface). FIG. 3G is a cross-sectional view of a sterile barrier 232. The sterile barrier 232 includes a convex upper support surface 204. Fluid channels 205 and 207 are positioned laterally of and below the support surface 204 for self-clearing or draining of fluids from the support surface 204 (for example, during an interventional procedure). The fluid channels 205 and 207 may extend axially at least a portion of the length of the sterile barrier.

FIGS. 3I, 3J, and 3K illustrate a sectional perspective view, a cross-sectional view, and a top sectional view, respectively, of a proximal end of the sterile barrier 232. As shown, in FIGS. 3I-3K, the sterile barrier 232 can include a trough 240 in communication with the fluid channels 205 and 207. The trough 240 can receive fluids from the channels 205 and 207 (for example, during an interventional procedure). The trough 240 may be positioned at least partially below the fluid channels 205 and 207 so that fluid within the channels 205 and 207 flows into the trough 240. In certain embodiments, the fluid channels 205 and 207 may be angled relative to a horizontal plane (for example, may decline from an end of the channel furthest from the trough 240 to the trough 240) so that fluid within the channels 205 and 207 is directed to the trough 240. For example, the channels 205 and 207 may increase in depth from an end of the channels furthest from the trough 240 to the trough 240. Alternatively, the sterile barrier 232 and/or support table may be positioned at an angle relative to a horizontal plane, during part of or an entirety of an interventional procedure, such that the end of the channels 205 and 207 furthest from the trough 240 is positioned higher than the trough 240. For example, the sterile barrier 232 and/or support table may be constructed or arranged in an angled arrangement so that an end of the sterile barrier 232 and/or support table opposite the trough 240 is positioned higher than the trough 240. Alternatively or additionally, a drive mechanism may temporarily tilt the sterile barrier 232 and/or support table so that an end of the sterile barrier 232 and/or support table opposite the trough 240 is positioned higher than the trough 240 (for example, by lifting an end of the sterile barrier and/or support table opposite the trough 240 or lowering an end of the sterile barrier 232 and/or support table at which the trough 240 is positioned) so that fluids within the channels 205 and 207 flow into the trough 240.

The trough 240 can include a drain hole 242. The trough 240 can be shaped, dimensioned, and/or otherwise configured so that fluid within the trough 240 empties to the drain hole 242. The drain hole 242 can include tubing, a barb fitting, and/or an on-off valve for removal of fluids from the trough 240. As shown in FIGS. 3I-3K, the trough 240 can be positioned at the proximal end of the sterile barrier 232. In alternate embodiments, the trough 240 may be positioned at a distal end of the sterile barrier 232. In some embodiments, the sterile barrier 232 can include a first trough 240 at the proximal end and a second trough 240 at the distal end. In some embodiments, the trough 240 can also be used as a wash basin.

A first channel 206 may extend axially at least a portion of the length of the sterile barrier 232. The channel 206 can have a sufficient length to hold the interventional devices, and sufficient width and depth to hold the corresponding hubs (for example, by providing support to prevent dislodgement of the hubs when forces are applied to the hubs). Optionally, a second channel 212 may be provided. The second channel 212 may be located on the same side or the opposite side of the upper support surface 204 from the first channel 206. FIG. 3G illustrates the channel 212 located on the opposite side of the support surface 204 from the channel 206. FIG. 3H is a cross-sectional view illustrating an alternate embodiment of the sterile barrier 232 in which the channel 212 is on the same side of the support surface 204 as the channel 206.

As shown in FIGS. 3G and 3H, the channels 206 and 212 can have generally triangular, wedge-shaped, or otherwise angled cross-sections, so as to hold the hubs at an angle relative to a horizontal plane. Holding the hubs at an angle relative to the horizontal plane can allow for smaller width of the sterile barrier 232.

Two or three or more additional recesses such as additional channels or wells may be provided, to hold additional medical devices or supplies that may be useful during the interventional procedure as well as to collect fluids and function as wash basins for catheters and related devices.

In some embodiments, the sterile barrier 232 can include one or more structural ribs 236. The sterile barrier 232 can further include one or more frame support bosses 228 and 238.

In the embodiment of the sterile barrier 232 shown in FIG. 3G, a width x₁ can be 14 in, about 14 in, between 12 in and 16 in, between 10 in and 18 in, or any other suitable width. In the embodiment of the sterile barrier 232 shown in FIG. 3H, the width x₁ can be 15 in, about 15 in, between 13 in and 17 in, between 11 in and 19 in, or any other suitable width. A height y₁ of the support surface 204 can be 0.125 in, about 0.125 in, between 0.1 and 0.15 in, or any other suitable height. In some embodiments, the support surface 204 can be recessed from a top surface 233 of the sterile barrier 232. A height y₂ between a bottom of the support surface 204 and the top surface 233 can be 0.5 in, about 0.5 in, between 0.25 in and 0.75 in, or any other suitable height. A width x₂ from a lateral edge of the channel 205 to a lateral edge of the channel 207 can be 5 in, about 5 in, between 4 in and 6 in, or any other suitable width. A width x₃ of the support surface 204 can be 4 in, about 4 in, between 3 in and 5 in, or any other suitable width. A height y₃ of the channel 206 and/or channel 212 can be 1.5 in, about 1.5 in, between 1 in and 2 in, or any other suitable height. A width x₄ of the channel 206 and/or channel 212 can be 3 in, about 3 in, between 2 in and 4 in, or any other suitable width. The channel 206 and/or channel 212 can be defined by an arc angle α of 90°, about 90°, between 80° and 100°, or any other suitable angle, and a radius of curvature of 0.125 in, about 0.125 in, between 0.1 and 0.15 in, or any other suitable radius of curvature. In certain embodiments, an arc angle α of 90° or about 90° may be used to hold a hub having a rectangular or generally rectangular cross-section. The support surface 204 can be defined by a radius of curvature of 13 in, about 13 in, between 11 in and 15 in, or any other suitable radius of curvature. The channel 205 and/or channel 207 can be defined by a radius of curvature of 0.25 in, about 0.25 in, between 0.15 in and 0.35 in, or any other suitable radius of curvature.

FIGS. 3L and 3M depict example dimensions of a hub 250 that may be used with the sterile barrier 232 as shown in FIGS. 3G-3K. The hub 250 may be any of the hubs described herein. In certain embodiments, the hub 250 can have a width w₁ of 3.75 in, about 3.75 in, between 3.25 in and 4.25 in, or any other suitable width. The hub 250 can have a height h₁ of 1.5 in, about 1.5 in, between 1.25 in and 1.75 in, or any other suitable height. Alternatively, the hub 250 can have a height h₂ of 2 in, about 2 in, between 1.75 in and 2.25 in, or any other suitable height. In some embodiments, the hub 250 can have a length L₁ of 2.5 in, about 2.5 in, between 2 in and 3 in or any other suitable length. Alternatively, the hub 250 can have a length L₂ of 4 in, about 4 in, between 3.25 in and 4.75 in, or any other suitable length.

In some embodiments, a top surface of the support table can include surface features that generally correspond to those of the sterile barrier 232. For example, the support table can include a convex surface configured to correspond to the shape, size, and location of the support surface 204 and/or one or more recesses configured to correspond to the shape, size, and location of the channels 205 and 207.

In alternate embodiments, a planar support surface (for example, support surface 104 of sterile barrier 32) can be positioned at an angle to a horizontal plane to facilitate the draining of fluids. In some embodiments, the sterile barrier and/or support table may be positioned, during part of or the entirety of an interventional procedure, at an angle to a horizontal plane to facilitate the draining of fluids. For example, the sterile barrier and/or support table may be constructed or arranged in an angled arrangement (for example, so that one lateral side of the planar support surface is positioned higher than the other lateral side of the planar support surface, the proximal end is higher than the distal end, or the distal end is higher than the proximal end) to facilitate the drainage of fluids. Alternatively or additionally, a drive mechanism may temporarily tilt the sterile barrier and/or support table (for example, so that one lateral side of the planar support surface is positioned higher than the other lateral side of the planar support surface, the proximal end is higher than the distal end, or the distal end is higher than the proximal end) to facilitate the drainage of fluids. For example, the drive mechanism may raise or lower one lateral side of the sterile barrier and/or support table, the proximal end of the sterile barrier and/or support table, and/or the distal end of the sterile barrier and/or support table.

In certain embodiments, a support surface (for example, support surface 104 of sterile barrier 32) can be positioned in a vertical configuration instead in the horizontal configuration shown, for example, in FIGS. 3A-3F. For example, the support surface 104 can be positioned at about 90 degrees (or any other suitable angle) from a horizontal plane (e.g., rotated 90 degrees about a long axis of the support surface 104 relative to the embodiment shown in of FIGS. 3A-3F). A vertical configuration may provide for easier interaction with the drive system 18 by a physician. A vertical configuration may also provide for a lower axis of catheter travel closer to a patient without adding standoff height to the drive system 18.

In some embodiments, the drive system 18 may be positioned, during part of or the entirety of an interventional procedure, at an angle to a horizontal plane to facilitate the draining of fluids. For example, the drive system 18 may be constructed or arranged in an angled arrangement (for example, so that one lateral side of the planar support surface is positioned higher than the other lateral side of the planar support surface, the proximal end is higher than the distal end, or the distal end is higher than the proximal end) to facilitate the drainage of fluids. Alternatively or additionally, a drive mechanism may temporarily tilt the drive system 18 (for example, so that one lateral side of the drive system 18 is positioned higher than the other lateral side of the drive system 18, the proximal end is higher than the distal end, or the distal end is higher than the proximal end) to facilitate the drainage of fluids. For example, the drive mechanism may raise or lower one lateral side of the system 18, the proximal end of the drive system 18, and/or the distal end of the drive system 18. In some embodiments, the drive system 18 may be angled so that it extends at an angle away from axis point 24 (for example, so that the proximal end is higher than the distal end), for example, to allow for clearance of a patient's fect.

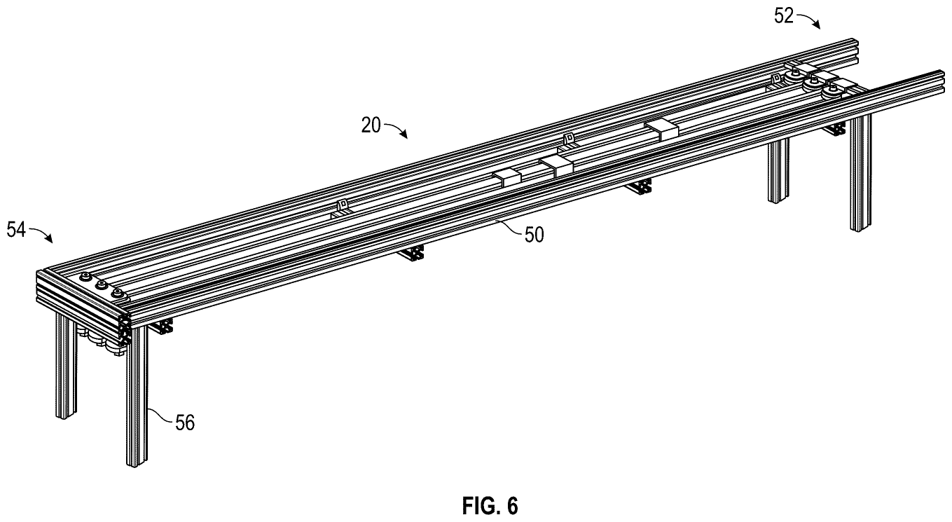

Referring to FIG. 4, hub 36 may represent any of the hubs previously described. Hub 36 includes a housing 38 which extends between a proximal end 40 and a distal end 42. An interventional device 44, which could be any of the interventional devices disclosed herein, extends distally from the hub 36 and into the patient 14 (not illustrated). A hub adapter 48 acts as a shuttle by advancing proximally or distally along a track in response to operator instructions or controller manipulations.

In any robotic control system embodiments disclosed herein, any of the embodiments of the hub adapters disclosed herein can be configured to move proximally or distally along a track (for example and without limitation, a linear track) in response to operator instructions or controller manipulations. For example and without limitation, any embodiments of the robotic control system and/or methods of controlling a movement of an interventional device through a sterile barrier disclosed herein can have one or more rack and pinion linear devices including any of the rack and pinion devices, components, and/or features disclosed in the following description.

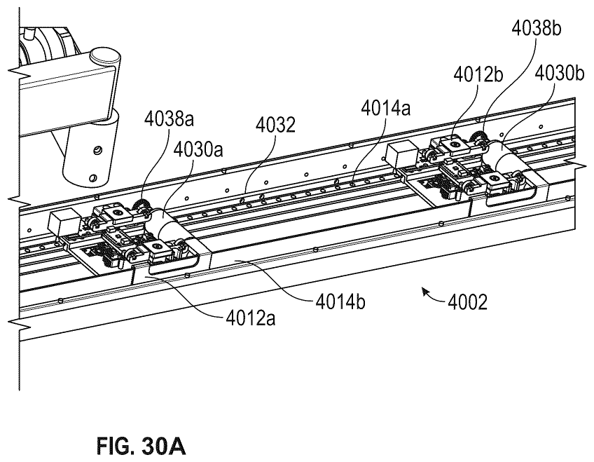

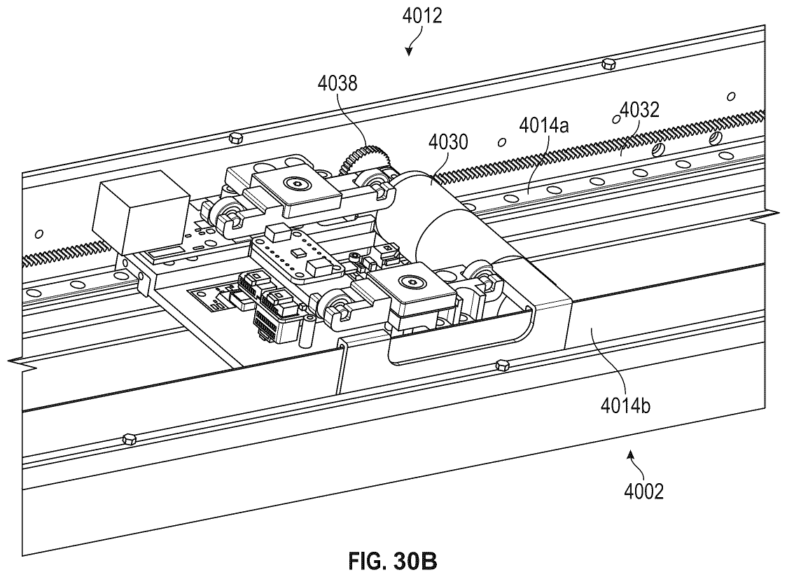

FIG. 30A shows a portion of an embodiment of a drive table having one or more rack and pinion actuators that can be used with any embodiments of the robotic control system disclosed herein. FIG. 30B shows an enlarged view of an embodiment of a rack and pinion actuator of the embodiment of the drive table shown in FIG. 30A that can be used with any embodiments of the robotic control system disclosed herein.

In some embodiments, one or more of a first hub adapter 4012a, a second hub adapter 4012b, a third hub adapter (not shown) and a fourth hub adapter (not shown) can be configured to move proximally and/or distally in the axial direction relative to the drive table 4002 via a linear actuator. In some embodiments, as in the illustrated embodiment, the linear actuator for one or more or all of the hub adapters can be or can include a rack and pinion linear actuator. In some embodiments, the one or more hub adapters can be configured to move in response to an input provided by a user of the robotic control system and/or in response to an automated command of the robotic control system.

FIG. 30B shows an embodiment of a hub adapter 4012, which may be any of the hub adapters described herein. With reference to FIG. 30B, some embodiments of the rack and pinion linear actuator can have a rack (or straight gear) 4032 and a pinion gear 4038 coupled to a shaft of the motor 4030 on the hub adapter 4012. For example and without limitations, with reference to FIG. 30A, the first hub adapter 4012, the second hub adapter 4012b, the third hub adapter (not shown), the fourth hub adapter (not shown), and/or any other hub adapter of the robotic control system or any combination of the foregoing hub adapters can have a motor and a pinion gear configured to engage the rack 4032. For example, the first hub adapter 4012a may have a pinon gear 4038a coupled to a shaft of a motor 4030a. The second hub adapter 4012b may have a pinion gear 4038b coupled to a shaft of a motor 4030b. In this configuration, any one or all of the hub adapters can have its own unique motor and pinion gear to allow for independent movement of each hub adapter.

With reference to FIG. 30A and FIG. 30B, in some embodiments of the robotic control system, the hub adapters can be coupled to and move axially along a rail or linear guide 4014a and/or a rail or linear guide 4014b. The linear guide 4014a and/or linear guide 4014b can guide and/or constrain the hub adapters to move axially (e.g., proximally and distally) along a linear path when moved by linear actuator. In any embodiments, the hub adapters (any combination of the hub adapters) can be configured to move in the axial direction relative to the drive table 4002 using a belt drive system, a plurality of wheels, a lead screw system, a ball screw system, a rack and pinion system, or any other suitable drive system or combination thereof.

Alternatively, in some embodiments, the hub adapter 4012 of FIG. 30B (which may be any of the hub adapters described herein) can have a plurality of wheels (e.g., low friction wheels) configured to move along one or more rails of a drive table 4002. In some embodiments, one side of the hub adapter 4012 can be spring loaded to facilitate coupling with the rails. In any embodiments disclosed herein, the table that supports the rails and/or the rails themselves can also be configured to move in a proximal or distal axial direction. Additionally, in some embodiments, the drive table 4002 may be a foldable drive table having two segments that are coupled at a hinge or joint that may be folded together.

In any embodiments disclosed herein, the first hub adapter 4012a can be distal to the second hub adapter 4012b, the second hub adapter 4012b can be distal to the third hub adapter, and the third hub adapter can be distal to the fourth hub adapter. In any embodiments, the second hub adapter 4012b can be axially aligned with the first hub adapter 4012a such that a first interventional device coupled with the first hub and a second interventional device coupled with the second hub are coaxially aligned when the first and second hubs are coupled with the first and second hub adapters 4012a, 4012b, respectively. Additionally, in some embodiments, the third hub adapter 4012 and the fourth hub adapter 4012 can be axially aligned with the first hub adapter 4012a such that a first interventional device coupled with the first hub, a second interventional device coupled with the second hub, a third interventional device coupled with the third hub, and a fourth interventional device coupled with the fourth hub are coaxially aligned with the first interventional device coupled with the first hub when the first, second, third, and fourth hubs are coupled (e.g., magnetically coupled) with the first, second, third, and fourth hub adapters, respectively. As described herein, the first interventional device, second interventional device, third interventional device, and fourth interventional device can be arranged in a concentric stack.

In addition to what is described above, any embodiments of the robotic control system and/or methods of controlling a movement of an interventional device disclosed herein can have any of the rack and pinion systems, components, and/or any features or details thereof disclosed in U.S. patent application Ser. No. 18/524,879 titled ROTATABLE DRIVE TABLE, filed on Nov. 30, 2023, which application and all of the rack and pinion systems, components, features, and/or details are hereby incorporated by reference in their entirety as if fully and explicitly set forth herein.

Any embodiments of the robotic control system and/or methods of controlling a movement of an interventional device disclosed herein can have any combination of rack and pinion actuator systems or devices, belt drive systems or devices, or other linear movement systems or devices configured to move one or more hub adapters and/or hubs proximally or distally along a linear track in response to operator instructions or controller manipulations. Additionally and without limitation, any and all embodiments of the drive table with or without the systems, components, and features related to the rack and pinion drive system disclosed in U.S. patent application Ser. No. 18/524,879 titled ROTATABLE DRIVE TABLE, filed on Nov. 30, 2023 can be used with and included in any of the embodiments of the robotic control system disclosed herein and are hereby incorporated by reference in their entirety as if fully and explicitly set forth herein.

In some embodiments, the hub adapter 48 can include at least one drive magnet 67 configured to couple with a driven magnet 69 carried by the hub 36. This provides a magnetic coupling between the drive magnet 67 and driven magnet 69 through the sterile barrier such that the hub 36 is moved across the top of the sterile barrier 32 in response to movement of the hub adapter 48 outside of the sterile field. Movement of the hub adapter is driven by a drive system carried by the support table and described in additional detail below. The hub adapter may act as a robotic drive for an interventional device coupled thereto.

To reduce friction in the system, the hub 36 may be provided with at least a first roller 53 and a second roller 55 which may be in the form of wheels or rotatable balls or drums. The rollers space the sterile barrier apart from the surface of the driven magnet 69 by at least about 0.02 centimeters (about 0.008 inches) and generally no more than about 0.08 centimeters (about 0.03 inches). In some implementations, the space is within the range of from about 0.03 centimeters (about 0.010 inches) and about 0.041 centimeters (about 0.016 inches). The space between the drive magnet 67 and driven magnet 69 is generally no more than about 0.38 centimeters (about 0.15 inches) and in some implementations is no more than about 0.254 centimeters (about 0.10 inches) such as within the range of from about 0.216 centimeters (about 0.085 inches) to about 0.229 centimeters (about 0.090 inches). The hub adapter 48 may similarly be provided with at least a first hub adapter roller 59 and the second hub adapter roller 63, which may be positioned opposite the respective first roller 53 and second roller 55 as illustrated in FIG. 4.

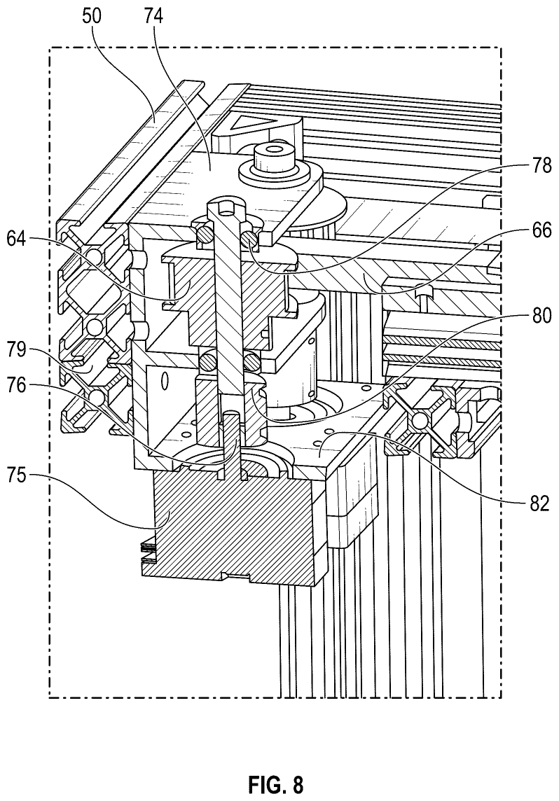

Referring to FIG. 6, there is illustrated one example of a low-profile linear drive support table 20. Support table 20 comprises an elongated frame 51 extending between a proximal end 52 and a distal end 54. At least one support table support 56 is provided to stabilize the support table 20 with respect to the patient (not illustrated). Support 56 may comprise one or more legs or preferably an articulating arm configured to allow movement and positioning of the frame 51 over or adjacent to the patient.

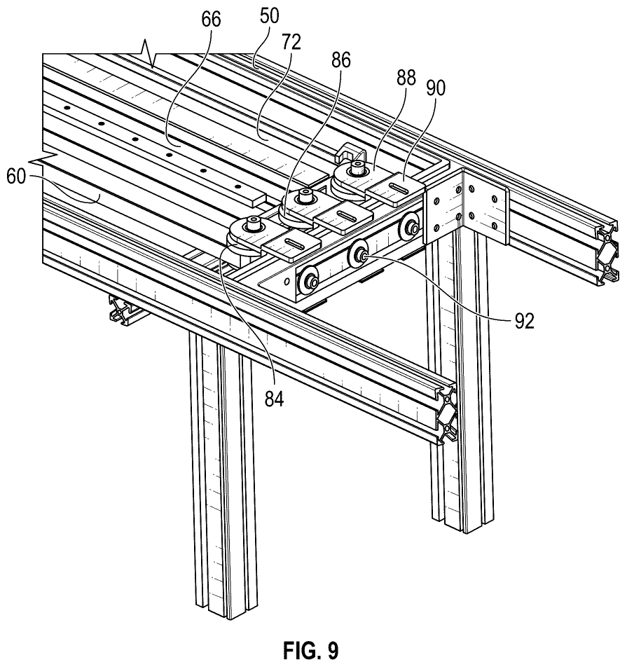

One example of a linear drive table 20 illustrated in FIG. 7 includes three distinct drives. However, two drives or four or more drives (e.g., up to eight drives) may be included depending upon the desired clinical performance. A first drive pulley 58 engages a first drive belt 60. A first hub adapter bracket 61 is secured to the first drive belt 60 such that rotation of the first drive pulley 58 causes rotation of the first drive belt 60 through an elongate closed loop path. The first hub adapter bracket 61 may be advanced in a proximal or distal direction along the longitudinal axis of the support table 20 depending upon the direction of rotation of the drive pully 58. In the illustrated implementation, the drive pulley 58 is provided with surface structures such as a plurality of drive pulley teeth 62 for engaging complementary teeth on the first drive belt 60.

A second drive pulley 64 may engage a second drive belt 66 configured to axially move a second hub adapter bracket 68 along an axial path on the support table 20. A third drive pulley 70 may be configured to drive a third drive belt 72, to advance a third hub adapter bracket 73 axially along the support table 20. Each of the hub adapter brackets may be provided with a drive magnet assembly discussed previously but not illustrated in FIG. 7, to form couplers for magnetically coupling to a corresponding driven magnet within the hub of an interventional device as has been discussed.

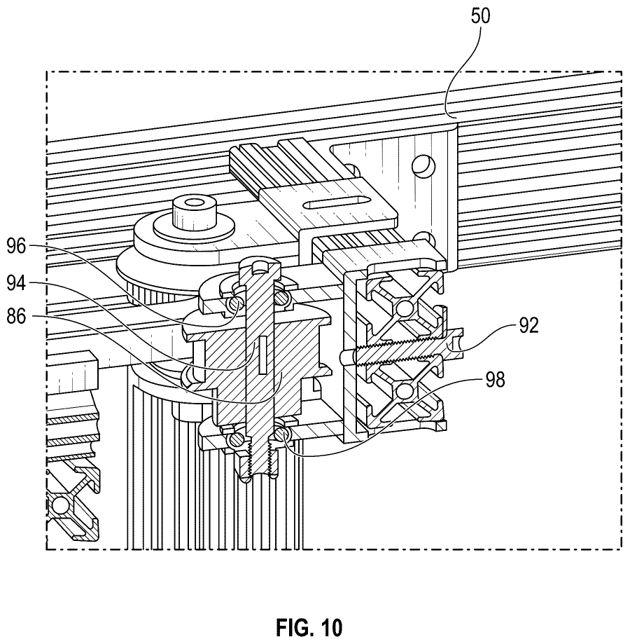

A detailed view of an embodiment of a drive system is shown in FIG. 8. A drive support 74 may be carried by the frame 51 for supporting the drive assembly. The second drive pulley 64 is shown in elevational cross section as rotationally driven by a motor 75 via a rotatable shaft 76. The rotatable shaft 76 may be rotatably carried by the support 74 via a first bearing 78, a shaft coupling 80 and second bearing 79. Motor 75 may be stabilized by a motor bracket 82 connected to the drive support 74 and or the frame 51. The belt drive assemblies for the first drive belt 60 and third drive belt 72 maybe similarly constructed and are not further detailed herein. In some embodiments, the drive systems described herein may be a rack and pinion drive table system that is foldable. In such embodiments, motors 75 may be attached to and move with the carriages.

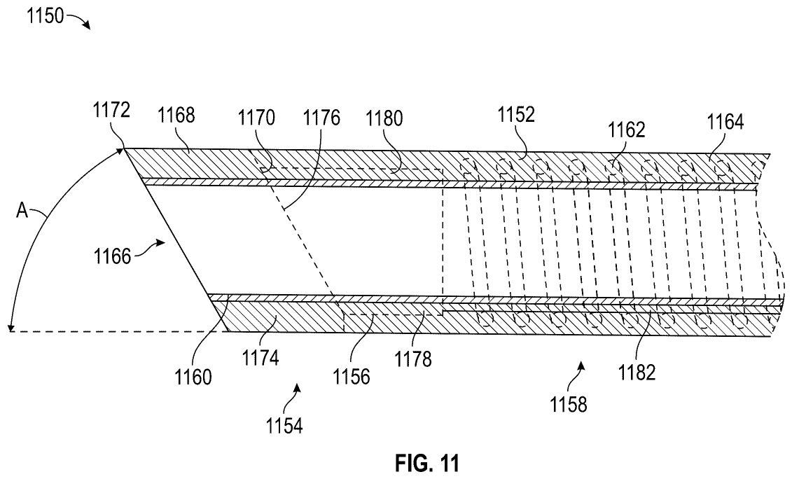

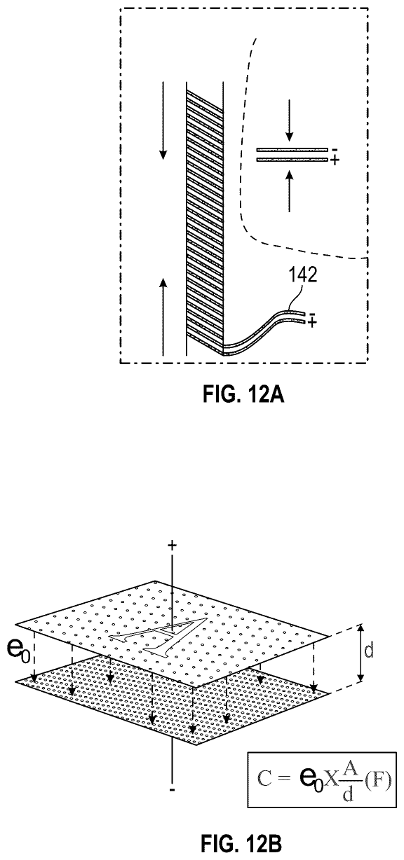

Referring to FIGS. 9 and 10, each of the first second and third drive belts extends around a corresponding first idler pulley 84, second idler pulley 86, and third idler pulley 88. Each idler pulley may be provided with a corresponding tensioning bracket 90, configured to adjust the idler pulleys in a proximal or distal direction in order to adjust the tension of the respective belt. Each tensioning bracket 90 is therefore provided with a tensioning adjustment 92 such as a rotatable screw.

As seen in FIG. 10, the second idler pulley 86, for example, may be carried by a rotatable shaft 94, rotatably secured with respect to the mounting bracket by a first bearing 96 and second bearing 98.

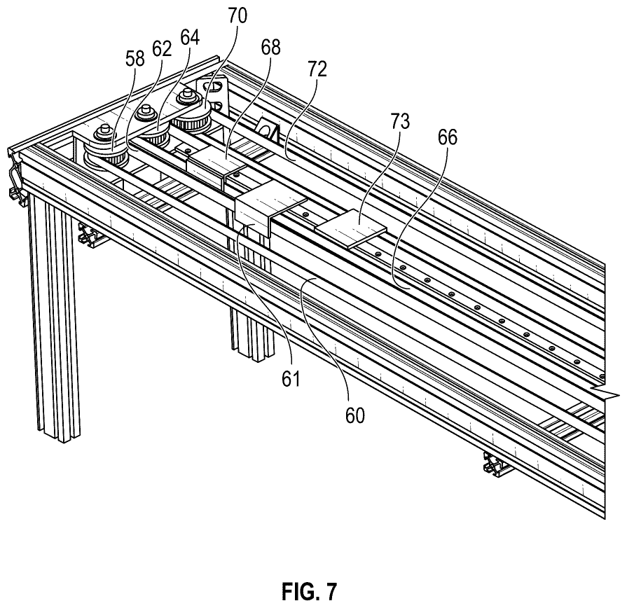

Any of the catheters illustrated, for example, in FIG. 5A, 5B or 11 generally comprise an elongate tubular body extending between a proximal end and a distal functional end. The length and diameter of the tubular body depends upon the desired application. For example, lengths in the area of from about 90 centimeters to about 195 centimeters or more are typical for use in femoral access percutaneous transluminal coronary applications. Intracranial or other applications may call for a different catheter shaft length depending upon the vascular access site.

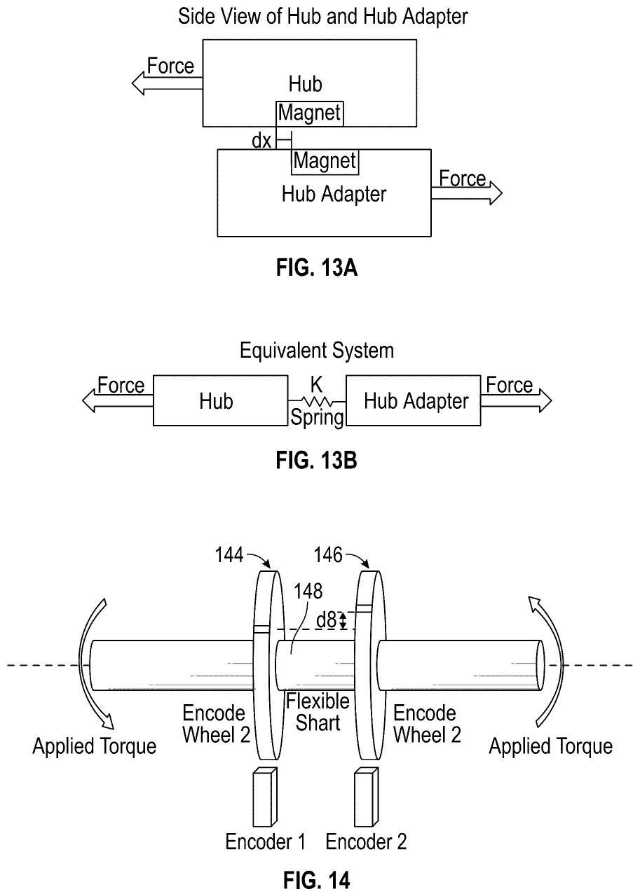

Any of the catheters disclosed herein may be provided with an inclined distal tip. Referring to FIG. 11, distal catheter tip 1150 comprises a tubular body 1152 which includes an advance segment 1154, a marker band 1156 and a proximal segment 1158. An inner tubular liner 1160 may extend throughout the length of the distal catheter tip 1150, and may comprise dip coated or extruded PTFE or other lubricious material.

A reinforcing element 1162 such as a braid and/or spring coil is embedded in an outer jacket 1164 which may extend the entire length of the catheter.

The advance segment 1154 terminates distally in an angled face 1166, to provide a leading side wall portion 1168 having a length measured between the distal end 130 of the marker band 1156 and a distal tip 1172. In some embodiments, the entire distal tip may be shaped to avoid snagging the tip in areas of arterial bifurcation. A trailing side wall portion 1174 of the advance segment 1154, has an axial length in the illustrated embodiment of approximately equal to the axial length of the leading side wall portion 1168 as measured at approximately 180 degrees around the catheter from the leading side wall portion 1168. The leading side wall portion 1168 may have an axial length within the range of from about 0.1 millimeters to about 5 millimeters and generally within the range of from about 1 to 3 millimeters. The trailing side wall portion 1174 may be equal to or at least about 0.1 or 0.5 or 1 millimeter or 2 millimeters or more shorter than the axial length of the leading side wall portion 1168, depending upon the desired performance.

The angled face 1166 inclines at an angle A within the range of from about 45 degrees to about 80 degrees from the longitudinal axis of the catheter. For certain implementations, the angle is within the range of from about 55 degrees to about 65 degrees from the longitudinal axis of the catheter. In one implementation, the angle A is about 60 degrees. One consequence of an angle A of less than 90 degrees is an elongation of a major axis of the area of the distal port which increases the surface area of the port and may enhance clot aspiration or retention. Compared to the surface area of the circular port (angle A is 90 degrees), the area of the angled port is generally at least about 105 percent, and no more than about 130 percent, in some implementations within the range of from about 110 percent and about 125 percent, and in one example is about 115 percent of the area of the corresponding circular port (angle A is 90 degrees).

In the illustrated embodiment, the axial length of the advance segment is substantially constant around the circumference of the catheter, so that the angled face 1166 is approximately parallel to the distal surface 1176 of the marker band 1156. The marker band 1156 has a proximal surface approximately transverse to the longitudinal axis of the catheter, producing a marker band 1156 having a right trapezoid configuration inside elevational view. A short sidewall 1178 is rotationally aligned with the trailing side wall portion 1174, and has an axial length within the range of from about 0.2 millimeters to about 4 millimeters, and typically from about 0.5 millimeters to about 2 millimeters. An opposing long sidewall 1180 is rotationally aligned with the leading side wall portion 1168. Long sidewall 1180 of the marker band 1156 is generally at least about 10 percent or 20 percent longer than short sidewall 1178 and may be at least about 50 percent or 70 percent or 90 percent or more longer than short sidewall 1178, depending upon desired performance. Generally, the long sidewall 1180 will have a length of at least about 0.5 millimeters or 1 millimeter and less than about 5 millimeters or 4 millimeters.

The marker band may be a continuous annular structure, or may have at least one and optionally two or three or more axially extending slits throughout its length. The slit may be located on the short sidewall 1178 or the long sidewall 1180 or in between, depending upon desired bending characteristics. The marker band may comprise any of a variety of radiopaque materials, such as a platinum/iridium alloy, with a wall thickness preferably no more than about 0.003 inches and in one implementation is about 0.001 inches.

The fluoroscopic appearance of the marker bands may be unique or distinct for each catheter size or type when a plurality of catheters is utilized so that the marker bands can be distinguishable from one another by a software algorithm. Distinguishing the marker bands of a plurality of catheters may be advantageous when the multiple catheters are used together, for example, in a multi catheter assembly or stack as described herein. In some embodiments, the marker band of a catheter may be configured so that a software algorithm can detect motion of the catheter tip.

The marker band zone of the assembled catheter may have a relatively high bending stiffness and high crush strength, such as at least about 50 percent or at least about 100 percent less than proximal segment 18 but generally no more than about 200 percent less than proximal segment 1158. The high crush strength may provide radial support to the adjacent advance segment 1154 and particularly to the leading side wall portion 1168, to facilitate the functioning of distal tip 1172 as an atraumatic bumper during transluminal advance and to resist collapse under vacuum. The proximal segment 1158 preferably has a lower bending stiffness than the marker band zone, and the advance segment 1154 preferably has even a lower bending stiffness and crush strength than the proximal segment 1158.

The advance segment 1154 may comprise a distal extension of the outer tubular jacket 1164 and optionally the inner liner 1160, without other internal supporting structures distally of the marker band 1156. Outer jacket 1164 may comprise extruded polyurethane, such as Tecothane®. The advance segment 1154 may have a bending stiffness and radial crush stiffness that is no more than about 50 percent, and in some implementations no more than about 25 percent or 15 percent or 5 percent or less than the corresponding value for the proximal segment 1158.

The catheter may further comprise an axial tension element or support such as a ribbon or one or more filaments or fibers for increasing the tension resistance and/or influencing the bending characteristics in the distal zone. The tension support may comprise one or more axially extending mono strand or multi strand filaments. The one or more tension element 1182 may be axially placed inside the catheter wall near the distal end of the catheter. The one or more tension element 1182 may serve as a tension support and resist tip detachment or elongation of the catheter wall under tension (e.g., when the catheter is being proximally retracted through a kinked outer catheter or tortuous or narrowed vasculature).

At least one of the one or more tension element 1182 may proximally extend along the length of the catheter wall from within about 1.0 centimeters from the distal end of the catheter to less than about 10 centimeters from the distal end of the catheter, less than about 20 centimeters from the distal end of the catheter, less than about 30 centimeters from the distal end of the catheter, less than about 40 centimeters from the distal end of the catheter, or less than about 50 centimeters from the distal end of the catheter.

The one or more tension element 1182 may have a length greater than or equal to about 40 centimeters, greater than or equal to about 30 centimeters, greater than or equal to about 20 centimeters, greater than or equal to about 10 centimeters, or greater than or equal to about 5 centimeters.

At least one of the one or more tension element 1182 may extend at least about the most distal 50 centimeters of the length of the catheter, at least about the most distal 40 centimeters of the length of the catheter, at least about the most distal 30 centimeters or 20 centimeters or 10 centimeters of the length of the catheter.

In some implementations, the tension element extends proximally from the distal end of the catheter along the length of the coil 24 and ends proximally within about 5 centimeters or 2 centimeters or less either side of a transition between a distal coil and a proximal braid. The tension element may end at the transition without overlapping with the braid.

The one or more tension element 1182 may be placed near or radially outside the inner liner 1160. The one or more tension element 1182 may be placed near or radially inside the braid and/or the coil. The one or more tension element 1182 may be carried between the inner liner 1160 and the helical coil, and may be secured to the inner liner or other underlying surface by an adhesive prior to addition of the next outer adjacent layer such as the coil. Preferably, the tension element 1182 is secured to the marker band 1156 such as by adhesives or by mechanical interference. In one implementation, the tension element 1182 extends distally beyond the marker band on a first (e.g., inside) surface of the marker band, then wraps around the distal end of the marker band and extends along a second (e.g., outside) surface in either or both a proximal inclined or circumferential direction to wrap completely around the marker band.

When more than one tension element 1182 or filament bundles are spaced circumferentially apart in the catheter wall, the tension elements 1182 may be placed in a radially symmetrical manner. For example, the angle between two tension elements 1182 with respect to the radial center of the catheter may be about 180 degrees. Alternatively, depending on desired clinical performances (e.g., flexibility, trackability), the tension elements 1182 may be placed in a radially asymmetrical manner. The angle between any two tension elements 1182 with respect to the radial center of the catheter may be less than about 180 degrees, less than or equal to about 165 degrees, less than or equal to about 135 degrees, less than or equal to about 120 degrees, less than or equal to about 90 degrees, less than or equal to about 45 degrees or, less than or equal to about 15 degrees.

The one or more tension element 1182 may comprise materials such as Vectran®, Kevlar®, Polyester®, Spectra®, Dyneema®, Meta-Para-Aramide®, or any combinations thereof. At least one of the one or more tension element 1182 may comprise a single fiber or a multi-fiber bundle, and the fiber or bundle may have a round or rectangular (e.g., ribbon) cross section. The terms fiber or filament do not convey composition, and they may comprise any of a variety of high tensile strength polymers, metals or alloys depending upon design considerations such as the desired tensile failure limit and wall thickness. The cross-sectional dimension of the one or more tension element 1182, as measured in the radial direction, may be no more than about 2 percent, 5 percent, 8 percent, 15 percent, or 20 percent of that of the catheter 10.

The cross-sectional dimension of the one or more tension element 1182, as measured in the radial direction, may be no more than about 0.03 millimeters (about 0.001 inches), no more than about 0.0508 millimeters (about 0.002 inches), no more than about 0.1 millimeters (about 0.004 inches), no more than about 0.15 millimeters (about 0.006 inches), no more than about 0.2 millimeters (about 0.008 inches), or about 0.38 millimeters (about 0.015 inches).

The one or more tension element 1182 may increase the tensile strength of the distal zone of the catheter before failure under tension (e.g., marker band detachment) to at least about 1 pound, at least about 2 pounds, at least about 3 pounds, at least about 4 pounds, at least about 5 pounds, at least about 6 pounds, at least about 7 pounds, at least about 8 pounds, or at least about 10 pounds or more.

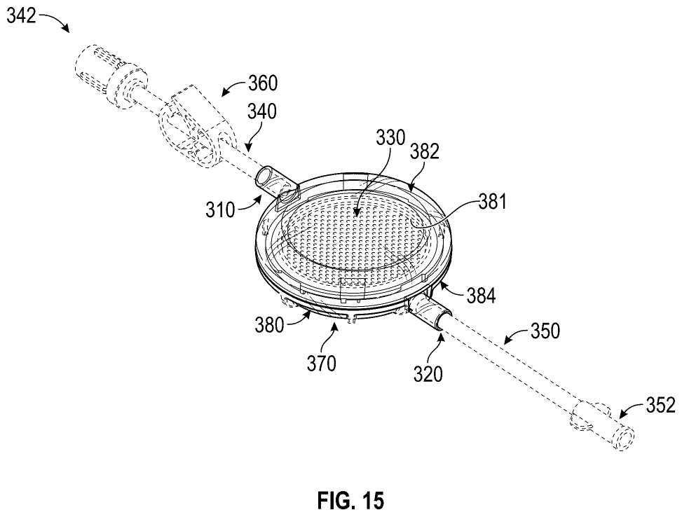

Any of a variety of sensors may be provided on any of the catheters, hubs, hub adapters, or table, depending upon the desired data. For example, in some implementations, it may be desirable to measure axial tension or compression force applied to the catheter such as along a force sensing zone. The distal end of the catheter would be built with a similar construction as illustrated in FIG. 11, with a helical coil distal section. But instead of using a single helical coil of nitinol wire, a first conductor 140 and second conductor 142 are wrapped into intertwined helical coils and electrically isolated from each other such as by the plastic/resin of the tubular body. See FIG. 12A. Each coil is in electrical communication with the proximal hub by a unique electrical conductor such as a conductive trace or proximal extension of the wire.

This construction of double, electrically isolated helical coils creates a capacitor. This is roughly equivalent to two plates of nitinol with a plastic layer between them, illustrated in FIG. 12B. The capacitance is inversely proportional to the distance between wires. The only variable that would be changing would be d, the distance between the plates. If an axial compressive force is applied to the catheter, the wires (e.g., conductor 140 and conductor 142) will move closer together, thus increasing the capacitance. If an axial tensile force is applied, the wires will get further apart, decreasing the capacitance. This capacitance can be measured at the proximal end of the catheter, giving a measurement of the force at the helical capacitor. Although referred to as a capacitor, this sensor is measuring the electrical interaction between the two coils of wire. There may be a measurable change in inductance or other resulting change due to applied axial forces.

At least a first helical capacitor may have at least one or five or ten or more complete revolutions of each wire. A capacitor may be located within the distal most 5 or 10 or 20 centimeters of the catheter body to sense forces experienced at the distal end. At least a second capacitor may be provided within the proximal most 5 or 10 or 20 centimeters of the catheter body, to sense forces experienced at the proximal end of the catheter.

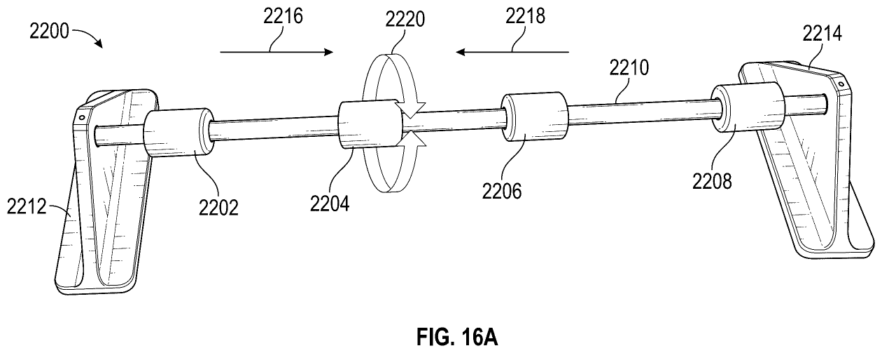

It may also be desirable to measure clastic forces across the magnetic coupling between the hub and corresponding hub adapter, using the natural springiness (compliance) of the magnetic coupling to measure the force applied to the hub. The magnetic coupling between the hubs and hub adapters creates a spring. When a force is applied to the hub, the hub will move a small amount relative to the hub adapter. See FIG. 13A. In robotics, this is called a series elastic actuator. This property can be used to measure the force applied from the hub adapter to the hub. To measure the force, the relative distance between the hub and the hub adapter (dx shown in FIG. 13A) is determined and characterize some effective spring constant k between the two components. See FIG. 13B.

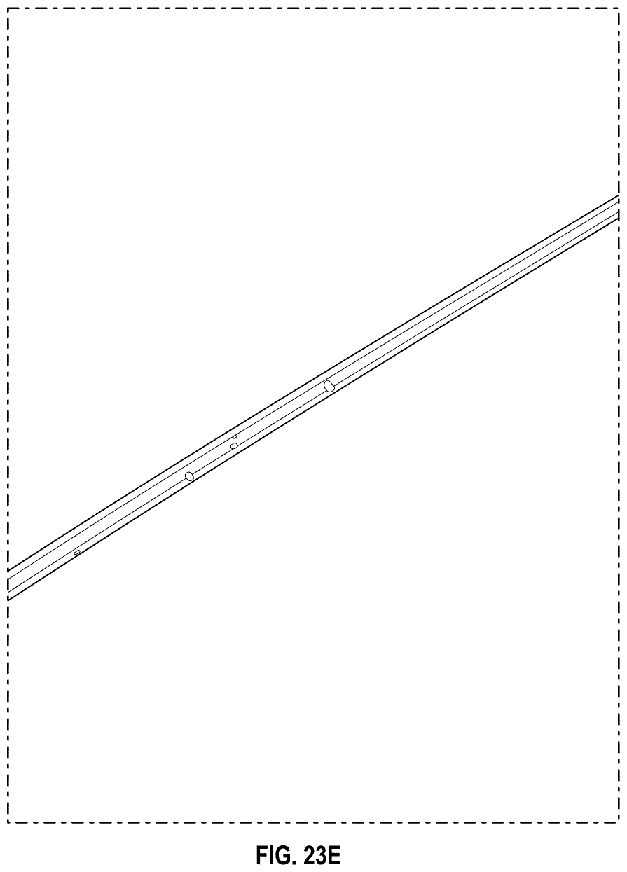

The relative distance could be measured in multiple different ways. One method for measuring the relative distance between the hub and hub adapter is a magnetic sensor (e.g., a Hall effect Sensor between hub and hub adapter). A magnet is mounted to either the hub or hub adapter, and a corresponding magnetic sensor is mounted on the other device (hub adapter or hub). The magnetic sensor might be a hall effect sensor, a magnetoresistive sensor, or another type of magnetic field sensor. Generally, multiple sensors may be used to increase the reliability of the measurement. This reduces noise and reduces interference from external magnetic fields. Additional details regarding magnetic sensors are described with respect to FIGS. 24-27.

Other non-contact distance sensors can also be used. These include optical sensors, inductance sensors, and capacitance sensors. Optical sensors would preferably be configured in a manner that avoids accumulation of blood or other fluid in the interface between the hubs and hub adapters. In some implementations, wireless (i.e., inductive) power may be used to translate movement and/or transfer information across the sterile barrier between a hub adapter and a hub, for example.

In some embodiments, the magnetic coupling between the hub and the hub adapter has a shear or axial break away threshold which may be about 300 grams or 1000 or 1300 grams or more. The processor can be configured to compare the axial force applied to the catheter to a preset axial trigger force which if applied to the catheter is perceived to create a risk to the patient. If the trigger force is reached, the processor may be configured to generate a response such as a visual, auditory or tactile feedback to the physician, and/or intervene and shut down further advance of the catheter until a reset is accomplished. An override feature may be provided so the physician can elect to continue to advance the catheter at forces higher than the trigger force, in a situation where the physician believes the incremental force is warranted.

Force and or torque sensing fiber optics (e.g., Fiber Bragg Grating (FBG) sensors) may be built into the catheter side wall to measure the force and/or torque at various locations along the shaft of a catheter or alternatively may be integrated into a guidewire. The fiber measures axial strain, which can be converted into axial force or torque (when wound helically). At least a first FBG sensor can be integrated into a distal sensing zone, proximal sensing zone and/or intermediate sensing zone on the catheter or guidewire, to measure force and or torque in the vicinity of the sensor.

It may also be desirable to understand the three-dimensional configuration of the catheter or guidewire during and/or following transvascular placement. Shape sensing fiber optics such as an array of FBG fibers to sense the shape of catheters and guidewires. By using multiple force sensing fibers that are a known distance from each other, the shape along the length of the catheter/guidewire can be determined.

A resistive strain gauge may be integrated into the body of the catheter or guidewire to measure force or torque. Such as at the distal tip and/or proximal end of the device.

Measurements of force and/or torque applied to the catheter or guidewire shafts can be used to determine applied force and/or torque above a safety threshold. When an applied force and/or torque exceeds a safety threshold, a warning may be provided to a user. Applied force and/or torque measurements may also be used to provide feedback related to better catheter manipulation and control. Applied force and/or torque measurements may also be used with processed fluoroscopic imaging information to determine or characterize distal tip motion.

In some embodiments, force and/or torque sensors can be incorporated into the hubs to indicate the force or torque being applied at the proximal end of the catheter or guidewire shafts. In some embodiments, the sensors may be multi-axis force/torque sensors (e.g., six-axis force/torque transducers). In some embodiments, the configurations described in Rafii-Tari et al., Objective Assessment of Endovascular Navigation Skills with Force Sensing, Annals of Biomedical Engineering 2017, the entirety of which is hereby incorporated by referenced herein, can be incorporated into the hubs and/or hub adapters.

Absolute position of the hubs (and corresponding catheters) along the length of the table may be determined in a variety of ways. For example, a non-contact magnetic sensor may be configured to directly measure the position of the hubs through the sterile barrier. The same type of sensor can also be configured to measure the position of the hub adapters. Each hub may have at least one magnet attached to it. In some embodiments, the robotic table can have a linear array of corresponding magnetic sensors going the entire length of the table. A processor can be configured to determine the location of the magnet along the length of the linear sensor array, and display axial position information to the physician.

The foregoing may alternatively be accomplished using a non-contact inductive sensor to directly measure the position of the hubs through the sterile barrier. Each hub or hub adapter may be provided with an inductive “target” in it. The robotic table may be provided with an inductive sensing array over the entire working length of the table. As a further alternative, an absolute linear encoder may be used to directly measure the linear position of the hubs or hub adapters. The encoder could use any of a variety of different technologies, including optical, magnetic, inductive, and capacitive methods.

In one implementation, a passive (no electrical connections) target coil may be carried by each hub. A linear printed circuit board (PCB) may run the entire working length of the table (e.g., at least about 1.5 meters to about 1.9 meters) configured to ping an interrogator signal which stimulates a return signal from the passive coil. The PCB is configured to identify the return signal and its location.

Axial position of the hub adapters may be determined using a multi-turn rotary encoder to measure the rotational position of the pulley, which directly correlates to the linear position of the hub adapter. Direct measurement of the location of the hub adapter may alternatively be accomplished by recording the number of steps commanded to the stepper motor to measure the rotational position of the pulley, which directly correlates to the linear position of the hub adapter.

The location of the catheters and guidewires within the anatomy may also be determined by processing the fluoroscopic image with machine vision, such as to determine the distal tip position, distal tip orientation, and/or guidewire shape. Comparing distal tip position or movement or lack thereof to commanded or actual proximal catheter or guidewire movement at the hub, may be used to detect a loss of relative motion, which may be indicative of a device shaft buckling, prolapse, kinking, or a similar outcome (for example, along the device shaft length inside the body (e.g., in the aorta) or outside the body between hubs. The processing may be done in real time to provide position/orientation data at up to 30 Hertz, although this technique would only provide data while the fluoroscopic imaging is turned on. In some embodiments, machine vision algorithms can be used to generate and suggest optimal catheter manipulations to access or reach anatomical landmarks, similar to driver assist. The machine vision algorithms may utilize data to automatically drive the catheters depending on the anatomy presented by fluoroscopy.

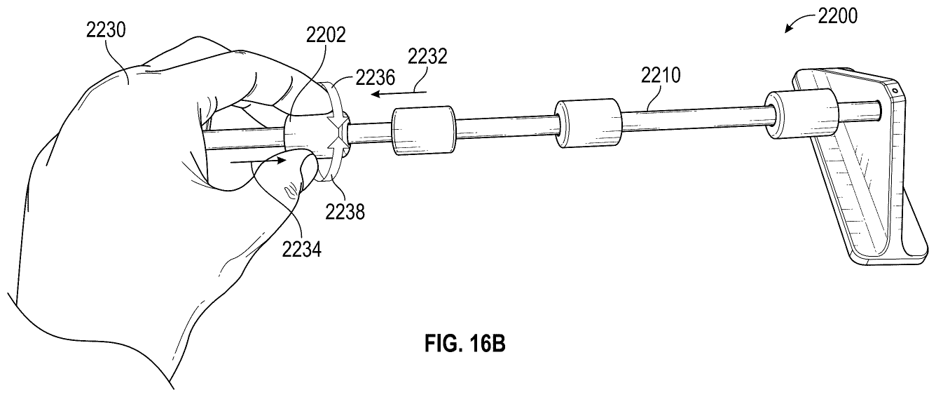

Proximal torque applied to the catheter or guidewire shaft may be determined using a dual encoder torque sensor. Referring to FIG. 14, a first encoder 144 and a second encoder 146 may be spaced axially apart along the shaft 148, for measuring the difference in angle over a length of flexible catheter/tube. The difference in angle is interpolated as a torque, since the catheter/tube has a known torsional stiffness. As torque is applied to the shaft, the slightly flexible portion of the shaft will twist. The difference between the angles measured by the encoders (de) tells us the torque. T=k*dθ, where k is the torsional stiffness.

Confirming the absence of bubbles in fluid lines may also be accomplished using bubble sensors, particularly where the physician is remote from the patient. This may be accomplished using a non-contact ultrasonic sensor that measures the intensity and doppler shift of the reflected ultrasound through the sidewall of fluid tubing to detect bubbles and measure fluid flow rate or fluid level. An ultrasonic or optical sensor may be positioned adjacent an incoming fluid flow path within the hub, or in a supply line leading to the hub. To detect the presence of air bubbles in the infusion line (that is formed of ultrasonically or optically transmissive material) the sensor may include a signal source on a first side of the flow path and a receiver on a second side of the flow path to measure transmission through the liquid passing through the tube to detect bubbles. Alternatively, a reflected ultrasound signal may be detected from the same side of the flow path as the source due to the relatively high echogenicity of bubbles.

Preferably, a bubble removal system is automatically activated upon detection of in line bubbles. A processor may be configured to activate a valve positioned in the flow path downstream of the bubble detector, upon the detection of bubbles. The valve diverts a column of fluid out of the flow path to the patient and into a reservoir. Once bubbles are no longer detected in the flow path and after the volume of fluid in the flow path between the detector and the valve has passed through the valve, the valve may be activated to reconnect the source of fluid with the patient through the flow path. In other embodiments, the bubble removal system can include a pump and control system upstream of the bubble detector for removal of in line bubbles. A processor may be configured to activate the pump upon detection of bubbles to reverse the fluid flow and clear the bubbles into a waste reservoir before reestablishing bubble free forward flow.

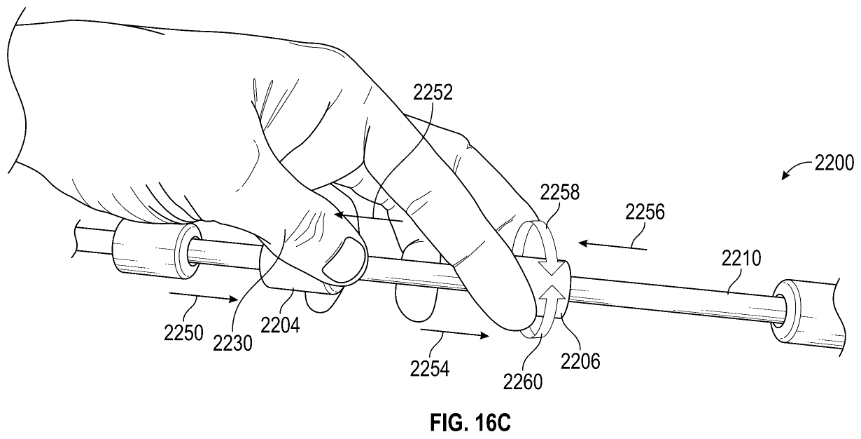

It may additionally be desirable for the physician to be able to view aspirated clot at a location within the sterile field and preferably as close to the patient as practical for fluid management purposes. This may be accomplished by providing a clot retrieval device mounted on the hub, or in an aspiration line leading away from the hub in the direction of the pump. Referring to FIG. 15, one example of a clot retrieval device 370 can include a body 380 enclosing a chamber 381 which communicates with a first port 310 and a second port 320.

In some embodiments, the body 380 includes a housing having a top portion 382 and a bottom portion 384. The body 380 may include a filter 330 positioned in the chamber 381 between the top portion 382, and the bottom portion 384. In some examples, the first port 310 is configured to connect to a first end of a first tube 340 that is fluidly connected to a proximal end of an aspiration catheter.

In an embodiment that is configured to be connected downstream from the hub, the first tube 340 includes a connector 342 positioned at a second end of the first tube 340 that is configured to engage or mate with a corresponding connector on or in communication with the hub. The first port 310 directly communicates with the chamber on the upstream (e.g., top side) of the filter, and the second port 320 directly communicates with the chamber on the downstream (e.g., bottom side) of the filter to facilitate direct visualization of material caught on the upstream side of the filter.

In an implementation configured for remote operation, any of a variety of sensors may be provided to detect clot passing through the aspiration line and/or trapped in the filter, such as an optical sensor, pressure sensor, flow rate sensor, ultrasound sensor or others known in the art.

In some embodiments, the second port 320 is configured to connect to a first end of a second tube 350 that is fluidly connected to an aspiration source (e.g., a pump). In some embodiments, the second tube 350 includes a connector 352 positioned at a second end of the second tube 350 that is configured to engage or mate with a corresponding connector on the pump.

In some examples, the system 300 can include an on-off valve 360 such as a clamp 360. The clamp 360 can be positioned in between the filter 330 and the patient, such as over the first tube 340 to allow the user to engage the clamp and provide flow control by isolating the patient from the clot retrieval device 370. Closing the valve 360 and operating the remote vacuum pump (not illustrated) causes the canister associated with the vacuum pump and the chamber 381 to reach the same low pressure. Due to the short distance and small line volume of the lumen between the chamber 381 end the distal end of the catheter, a sharp negative pressure spike is experienced at the distal end of the catheter rapidly following opening of the valve 360. Additional details are disclosed in U.S. Pat. No. 11,259,821 issued Mar. 1, 2022 to Buck et al., entitled Aspiration System with Accelerated Response, the entirety of which is hereby expressly incorporated by reference herein. In some embodiments, a vacuum may be cycled against a clot to retrieve the clot. The vacuum may be automatically and robotically controlled to remove the clot.

The body 380 can have a top surface spaced apart from a bottom surface by a tubular side wall. In the illustrated implementation, the top and bottom surfaces are substantially circular, and spaced apart by a cylindrical side wall. The top surface may have a diameter that is at least about three times, or five times or more than the axial length (transverse to the top and bottom surfaces) of the side wall, to produce a generally disc shaped housing. Preferably at least a portion of the top wall is optically transparent to improve clot visualization once it is trapped in the clot retrieval device 370. Additional details may be found in U.S. Patent Application No. 63/256,743, the entirety of which is hereby incorporated by reference herein.

In some examples, the body 380 can include a flush port (not illustrated) that is configured to allow the injection of an optically transparent media such as air, saline or other fluid into the chamber 381 to clear an optical path between the window and the filter to improve clot visualization once it is trapped in the filter 330.

The foregoing represents certain specific implementations of a drive table and associated components and catheters. A wide variety of different drive table constructions can be made, for supporting and axially advancing and retracting two or three or four or more drive magnet assemblies to robotically drive interventional devices, fluid elements, and electrical umbilical elements for communicating electrical signals and fluids to the catheter hubs, as will be appreciated by those of skill in the art in view of the disclosure herein. Additional details may be found in U.S. patent application Ser. No. 17/527,393, the entirety of which is hereby incorporated by reference herein.

While the foregoing describes robotically driven interventional devices and manually driven interventional devices, the devices may be manually driven, robotically driven, or a combination of both manually and robotically driven interventional devices, as will be appreciated by those of skill in the art in view of the disclosure herein.

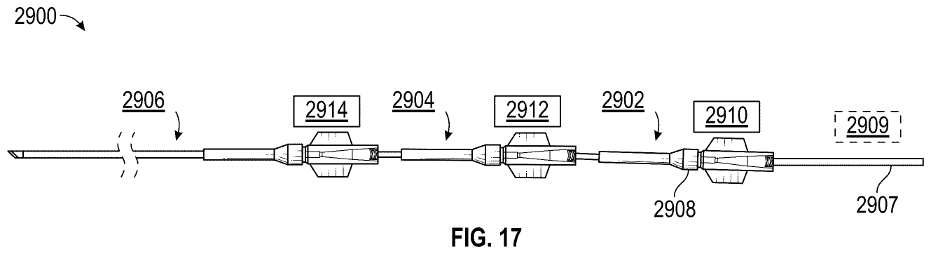

FIGS. 16A-16C illustrate an example control mechanism 2200 for manipulating interventional devices driven by (or otherwise associated with) respective hubs. For example, each hub may be manipulated and/or otherwise moved using at least one control installed in control mechanism 2200. Each control may be adapted to move a unique hub and associated interventional device during an interventional procedure.

As shown in FIG. 16A, the control mechanism 2200 include a first control 2202, a second control 2204, a third control 2206, and a fourth control 2208. More or fewer controls may be provided, depending upon the intended interventional devices configuration. Each control 2202-2208 is movably carried on a shaft 2210 that is coupled to a distal bracket 2212 and to a proximal bracket 2214. The controls 2202-2208 may advance distally or retract proximally on the shaft 2210, as indicated by arrow 2218 and arrow 2216. In addition, each control 2202-2208 may also be rotated about the shaft 2210, as indicated by arrow 2220. Each control movement may trigger a responsive movement in a corresponding hub adapter on the support table, which may in turn drive movement of a corresponding hub as has been discussed.

The control mechanism 2200 may be positioned on or near to a patient support table having a set of hubs and catheters/interventional devices. In some implementations, the control mechanism 2200 may be positioned remote from the support table such as behind a radiation shield or in a different room or different geographical location in a telemedicine implementation.

Each control 2202-2208 may correspond to and drive movement of a hub and/or a hub and interventional device combination. For example, the control 2202 may be configured to drive hub 30 (FIG. 3F) to move an interventional device such as an 0.088 inch guide catheter corresponding to the hub 30. Similarly, the control 2204 may be configured to drive hub 28 (122) to move an interventional device such as an 0.071 inch procedure catheter. The control 2206 may be configured to drive hub 126 to move an interventional device such as a steerable access catheter. The control 2208 may be configured to drive hub 26 to axially and rotationally move an interventional device such as a guidewire.

FIG. 16B illustrates an example of manually manipulating the control 2202 on control mechanism 2200. In operation, if the user 2230 moves the control 2202 axially along shaft 2210 and distally, as shown by arrow 2232, a corresponding coupled hub and/or interventional device may move responsively in the same direction by a same or scaled amount. If the user 2230 rotates the control 2202 about the shaft 2210 and advances the control proximally, as shown by arrow 2234, a corresponding coupled interventional device will responsively move rotationally and proximally by a same or scaled amount. If the user 2230 moves the control 2202 rotationally about the shaft 2210, as shown by arrow 2236 or arrow 2238, a corresponding coupled hub will drive the corresponding interventional device rotationally in the same direction and/or by a same or scaled amount.

Other axes and degrees of freedom may be defined to enable control 2202 to perform movements that may be translated to movement of hubs and/or interventional devices. For example, the control mechanism may be provided with one or more deflection controls configured to initiate a lateral deflection in a deflection zone on the corresponding interventional device.

Axial movement of a control may be configured to move the coupled hub on a 1:1 basis, or on a non 1:1 scaled basis. For example, if the user 2230 advances the control 2022 about 5 millimeters distally along the shaft 2210, then the corresponding hub may responsively move 5 millimeters in the distal direction.

If the user 2230 rotates the control 2022 about its rotational axis by 5 degrees, the coupled hub will cause the corresponding interventional device to rotate on a 1:1 basis or on a non 1:1 scaled basis. The scaled amount may be selected to reduce or increase the amount of distance and rotation that a hub and/or interventional device moves in accordance with the control movement.

In some implementations, the scaled amount described herein may be determined using a scale factor. The scale factor may apply to one or both translational and rotational movement. In some implementations, a first scale factor is selected for translational movement and a second scale factor, different than the first scale factor, is selected for rotational movement. The axial scaling factor may drive proximal catheter movement at a faster speed than distal catheter movement for a given proximal or distal manipulation of the control.

The rotational scale factor may be 1:1 while the axial scale factor may move the hub by a greater distance than movement of the control such that hub travel to control travel is at least about 2:1 or 5:1 or 10:1 or more depending upon the desired axial length of the control assembly.

The control mechanism 2200 may be configured to enable the clinician to adjust the scale factor for different parts of the procedure. For example, distal advance of the procedure catheter and access catheter through the guide catheter and up to the selected ostium may desirably be accomplished in a ‘fast’ mode. But more distal travel into the neuro vasculature may desirably be accomplished in a relatively slow mode by actuation of a speed control.

In another implementation, one or more controls may be configured to progressively drive advance or retraction speeds of the corresponding hub and associated catheter. For example, distal control 2202 may drive the guide catheter. A slight distal movement of the control 2202 may advance the guide catheter distally at a slow speed, while advancing the control 2202 by a greater distance distally increases the rate of distal travel of the guide catheter.

Controlling the speed of the corresponding hubs either axially or both axially and rotationally may enhance the overall speed of the procedure. For example, advance of the various devices from the femoral access point up to the aortic arch may desirably be accomplished at a faster rate than more distal navigation closer to the treatment site. Also proximal retraction of the various devices, particularly the guidewire, access catheter and procedure catheter may be desirably accomplished at a relatively higher speeds than distal advance.

FIG. 16C illustrates another example of manually manipulating a control on the control mechanism 2200 to move hubs and/or other interventional devices. In some implementations, two or more controls 2202-2208 may be moved in combination to trigger movement of one or more hubs and/or related interventional devices. In the depicted example, the user 2230 moves control 2204 and control 2206 in combination (e.g., sequentially, simultaneously) such as to simultaneously move the 0.088 guide catheter and the 0.071 aspiration catheter as a unit. Example movement of control 2204 may include axial proximal movement in the directions shown by arrows 2250. Sequentially or simultaneously, the user 2230 may move control 2206 axially in either of the directions shown by arrows 2254 and 2256 while also moving control 2206 rotationally in either of the directions shown by arrows 2258 and 2260.

In some implementations, each control mechanism and/or additional controls (not shown) may be color coded, shaped coded, tactile coded, or other coding to indicate to the user 2230 which color is configured to move which hub or interventional device. In some implementations, the control color coding may also be applied to the hubs and/or interventional devices such that a user may visually match a particular hub/device with a particular control.

In some implementations, other control operations beyond translational movement and rotational movement may be carried out using controls 2202-2208. For example, controls 2202-2208 may be configured to drive a shape change and/or stiffness change of a corresponding interventional device. Controls 2202-2208 may be toggled between different operating modes. For example, controls 2202-2208 may be toggled between movement driven by acceleration and velocity to movement that reflects actual linear displacement or rotation.

In some implementations, the control mechanism 2200 may be provided with a visual display or other indicator of the relative positions of the controls which may correspond the relative positions of the interventional devices. Such displays may depict any or all movement directions, instructions, percentage of movements performed, and/or hub and/or catheter indicators to indicate which device is controlled by a particular control. In some implementations, the display may depict applied force or resistance encountered by the catheter or other measurement being detected or observed by a particular hub or interventional component.

In some implementations, the control mechanism 2200 may include haptic components to provide haptic feedback to a user operating the controls. For example, if the control 2202 is triggering movement of a catheter and the catheter detects a large force at the tip, the control 2202 may generate haptic feedback to indicate to the user to stop or reverse a performed movement. In some implementations, haptic feedback may be generated at the control to indicate to the user to slow or speed a movement using the control. In some implementations, haptics may provide feedback on a large torsional strain buildup that might precede an abrupt rotation, or a large axial force buildup that may be a prelude to buckling of the catheter.

The systems described herein may compare an actual fluoroscopic image position to an input displacement from the controller. A static fluoroscopic image of the patient may be captured in which the patient's vasculature is indexed relative to bony landmarks or one or more implanted soft tissue fiducial markers. Then a real time fluoroscopic image may be displayed as an overlay, aligned with the static image by registration of the fiducial markers. Visual observation of conformance of the real time movement with the static image, assisted by detected force data can help confirm proper navigation of the associated catheter or guidewire. The systems described herein can also display a comparison of an input proximal mechanical translation of a catheter or guidewire and a resulting distal tip output motion or lack thereof. A loss of relative motion at the distal tip may indicate shaft buckling, prolapse, kinking, or a similar outcome, either inside or outside the body. Such a comparison may be beneficial when the shaft buckling, prolapse, kinking, or similar outcome occurs outside of a current fluoroscopic view.

FIG. 17 illustrates a side elevational schematic view of a multi catheter interventional device assembly 2900 for combined supra-aortic access and/or neurovascular site access and procedure (e.g., aspiration), as described herein. The multi catheter assembly 2900 may be configured for either a manual or a robotic procedure.

The interventional device assembly 2900 includes an insert or access catheter 2902, a procedure catheter 2904, and a guide catheter 2906. Other components are possible including, but not limited to, one or more guidewires (e.g., optional guidewire 2907), one or more guide catheters, an access sheath and/or one or more other procedure catheters and/or associated catheter (control) hubs. In some embodiments, the assembly 2900 may also be configured with an optional deflection control 2908 for controlling deflection of one or more catheters of assembly 2900.

In operation, the multi-catheter assembly 2900 may be used without having to exchange hub components. For example, in the two stage procedure disclosed previously, a first stage for achieving supra-aortic access, includes mounting an access catheter, guide catheter and guidewire to the support table. Upon gaining supra aortic access, the access catheter and guidewire were typically removed from the guide catheter. Then, a second catheter assembly is introduced through the guide catheter after attaching a new guidewire hub and a procedure catheter hub to the corresponding drive hub adapter on the support table.

The single multi catheter assembly 2900 of FIG. 17 is configured to be operated without having to remove hubs and catheters and without the addition of additional assemblies and/or hubs. Thus, the multicomponent access and procedure configuration of assembly 2900 may utilize a guidewire 2907 manufactured to function as an access guidewire and a navigation guidewire to allow for sufficient access and support, and navigation to the particular distal treatment site. In a non-limiting example configured for robotic implementation, a catheter assembly may include a guidewire hub (e.g., guidewire hub 2909 or guidewire hub 26 positioned on a drive table and to the right of catheter 2902), an insert or access catheter hub 2910, a procedure catheter hub 2912, a guide catheter hub 2914 and corresponding catheters. In certain embodiments, one or more of the hubs may include or be coupled to a hemostasis valve (e.g., a rotating hemostasis valve) to accommodate introduction of interventional devices therethrough. Additional details regarding hemostasis valves are included in U.S. patent application Ser. No. 17/879,614, entitled Multi Catheter System With Integrated Fluidics Management, filed Aug. 2, 2022, which is hereby expressly incorporated in its entirety herein

Once access above the aortic arch has been achieved, the insert or access catheter 2902 (associated with insert catheter hub 2910) may be parked in the vicinity of a carotid artery ostia and the remainder or a subset of the catheter assembly may be guided more distally toward a particular site (e.g., a clot site, a surgical site, a procedure site, etc.).

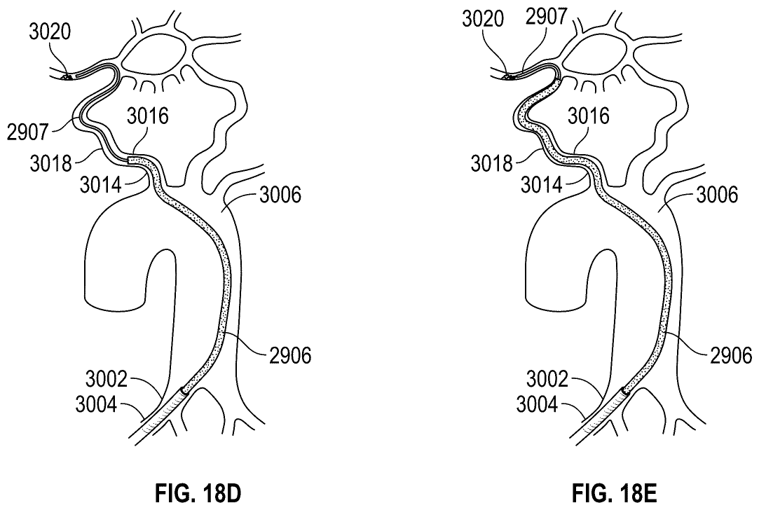

In some embodiments, other smaller procedure catheters may also be added and used at the site. As used herein for catheter assembly 2900, in a robotic configuration of assembly 2900, the catheter 2906 may function as a guide catheter. The catheter 2904 may function as a procedure (e.g., aspiration) catheter. In some embodiments, the catheter 2906 may function to perform aspiration in addition to functioning as a guide catheter, either instead of or in addition to the catheter 2904. The access catheter 2902 may have a distal deflection zone and can function to access a desired ostium. One of skill in the art will appreciate from FIGS. 18A-18E that either manual manipulation or robotic manipulation of the multi catheter stack are contemplated herein.

In some embodiments, the catheter assembly 2900 (or other combined catheter assemblies described herein) may be driven as a unit to a location. However, each catheter (or guidewire) component may instead be operated and driven independent of one another to the same or different locations.

In a non-limiting example, the catheter assembly 2900 may be used for a diagnostic angiogram procedure. In some embodiments, the assembly 2900 may include only the guidewire 2907 and access catheter 2902 (in the form of a diagnostic angiographic catheter) for performing the diagnostic angiogram procedure or only the guidewire 2907 and the access catheter 2902 may be utilized during the procedure. Alternatively, the guide catheter 2906 and procedure catheter 2904 may be retracted proximally to expose the distal end of the access catheter 2902 (e.g., a few centimeters of the distal end of the access catheter) to perform the diagnostic angiography.

As shown in FIG. 17, the guide catheter 2906, procedure catheter 2904, access catheter 2902, and guidewire 2907 can be arranged concentrically. In certain embodiments, the guide catheter 2906 may be a ‘large bore’ guide catheter or access catheter having a diameter of at least about 0.075 or at least about 0.080 inches in diameter. The procedure catheter 2904 may be an aspiration catheter having a diameter within the range of from about 0.060 to about 0.075 inches. The access catheter 2902 may be a steerable catheter with a deflectable distal tip, having a diameter within the range of from about 0.025 to about 0.050 inches. The guidewire 2907 may have a diameter within the range of from about 0.014 to about 0.020 inches. In one example, the guide catheter 2906 may have a diameter of about 0.088 inches, the procedure catheter 2904 about 0.071 inches, the access catheter 2902 about 0.035 inches, and the guidewire 2907 may have a diameter of about 0.018 inches.

FIGS. 18A-18E depict an example sequence of steps of introducing a multi-catheter assembly configured to achieve access all the way to the clot, either manually or robotically. FIGS. 18A-18E may be described using the interventional device assembly of FIG. 17. Other combinations of catheters may be substituted for the interventional device assembly, as will be appreciated by those of skill in the art in view of the disclosure herein.

Referring to FIG. 18A, the three catheter interventional device assembly 2900 is shown driven through an introducer sheath 3002, up through the iliac artery 3004 and into the descending aorta. Next, the access catheter 2902, the procedure catheter 2904 (e.g., 0.071 inch) and the guide catheter 2906 (e.g., 0.088 inch) are tracked up to the aortic arch 3006, as shown in FIG. 18B. Here, the distal end of the guide catheter 2906 may be parked below the aortic arch 3006 and the procedure catheter 2904, access catheter 2902 (positioned within the procedure catheter 2904 and not visible in FIG. 18B), and a guidewire 2907 can be driven into the ostium (e.g., simultaneously or separately). In some embodiments, the access catheter 2902 is advanced out of the procedure catheter 2904 and the guide catheter 2906 to engage the ostium first. After the distal end of the access catheter 2902 is positioned within the desired ostium, the guidewire 2907 can be advanced distally into the ostium to secure access. After the access catheter 2902 and guidewire 2907 are positioned within the desired ostium, the procedure catheter 2904 and/or guide catheter 2906 can be advanced into the ostium (and, in some embodiments, beyond), while using the support of the access catheter 2902 and/or guidewire 2907 to maneuver through the aorta and into the ostium. In the embodiment shown in FIG. 18B, the procedure catheter 2904 has been advanced into the ostium while the guide catheter 2906 has remained parked below the aortic arch 3006.

Referring to FIG. 18C, the guidewire 2907 may be distally advanced and the radiopacity of the guidewire 2907 may be used to confirm under fluoroscopic imaging that access through the desired ostia has been attained. The guidewire 2907 engages the origin of the brachiocephalic artery 3014. The guidewire 2907 is then advanced up to the petrous segment 3018 of the internal carotid artery 3016.

Referring to FIG. 18D, the guide catheter 2906 and the procedure catheter 2904 (positioned within the guide catheter 2906 and not visible in FIG. 18D) are both advanced (e.g., simultaneously or sequentially) over the guidewire 2907 and over the insert or access catheter 2902 (positioned within the procedure catheter 2904 and not visible in FIG. 18D) while the access catheter 2902 remains at the ostium for support. The guidewire 2907 may be further advanced past the petrous segment 3018 to the site of the clot 3020, such as the. Ml segment.

Referring to FIG. 18E, the guide catheter 2906 and the procedure catheter 2904 (positioned within the guide catheter 2906 and not visible in FIG. 18E) are advanced (e.g., simultaneously or sequentially) to position the distal tip of the procedure catheter 2904 at the procedure site, for example on the face of the clot 3020. The guidewire 2907 and access catheter 2902 (positioned within the procedure catheter 2904 and not visible in FIG. 18E) are removed, and aspiration of the clot 3020 commences through the procedure catheter 2904. That is, the guidewire 2907 and the access catheter 2902 are proximally retracted to allow aspiration through the procedure catheter 2904. After aspiration of the clot, the procedure catheter 2904 and guide catheter 2906 can be removed (e.g., simultaneously or sequentially). For example, in some embodiments, the procure catheter 2904 may be removed before removing the guide catheter 2906.

The catheter assembly 2900 may be used to perform a neurovascular procedure, as described in FIGS. 18A-18E. For example, the neurovascular procedure may be a neurovascular thrombectomy. The steps of the procedure may include providing an assembly that includes at least a guidewire, an access catheter, a guide catheter, and a procedure catheter. For example, the catheter assembly 2900 includes a guidewire 2907, an access (e.g., insert) catheter 2902, a guide catheter 2906, and at least one procedure catheter 2904. The procedure catheter 2904 may include an aspiration catheter, an embolic deployment catheter, a stent deployment catheter, a flow diverter deployment catheter, a diagnostic angiographic catheter, a stent retriever catheter, a clot retriever catheter, a balloon catheter, a catheter to facilitate percutaneous valve repair or replacement, an ablation catheter, and/or an RF ablation catheter or guidewire.

The neurovascular procedure may further include steps of coupling the assembly to a non-robotic or a robotic drive system, and driving the assembly to achieve supra-aortic access. The steps may further include driving a subset of the assembly to a neurovascular site, and performing the neurovascular procedure using a subset of the assembly. The subset of the assembly may include the guidewire, the guide catheter, and the procedure catheter.

Each of the guidewire 2907, the access catheter 2902, the guide catheter 2906, and the procedure catheter 2904 is configured to be adjusted by a respective hub. For example, the guidewire 2907 may include (or be coupled to) a hub installed on one of the tray assemblies described herein. Similarly, the access catheter 2902 may be coupled to catheter hub 2910. The guide catheter 2906 may be coupled to the guide catheter hub 2914. The procedure catheter 2904 may be coupled to the procedure catheter hub 2912.

In general coupling of the assembly may include magnetically coupling a first hub 2909 on the guidewire 2907 to a first drive magnet, magnetically coupling a second hub 2910 on the access catheter 2902 to a second drive magnet, magnetically coupling a third hub 2912 on the procedure catheter 2904 to a third drive magnet, and magnetically coupling a fourth hub 2914 on the guide catheter 2906 to a fourth drive magnet. In general, the first drive magnet, the second drive magnet, the third drive magnet, and the fourth drive magnet are each independently movably carried by a drive table, as described with respect to tray assemblies and controls described herein. In some embodiments, the first drive magnet, the second drive magnet, the third drive magnet, and the fourth drive magnet are coupled (e.g., to their respective catheter hubs) through a sterile barrier (e.g., a sterile and fluid barrier) and independently movably carried by a drive table having a plurality of driven magnets. In some embodiments, two or more drive magnets can be tethered or otherwise coupled together to move as a unit in response to commands from a single controller tethered or otherwise coupled to one of the drive magnets.

In some implementations, the steps of performing the neurovascular procedure may include driving the assembly in response to movement of each of the hub adapters along a support table until the assembly is positioned to achieve supra-aortic vessel access. The hub adapters may include, for example, a coupler/carriage that acts as a shuttle by advancing proximally or distally along a track in response to operator instructions. The hub adapters described herein may each include at least one drive magnet configured to couple with a driven magnet carried by the respective hub. This provides a magnetic coupling between the drive magnet and driven magnet through the sterile barrier such that the respective hub is moved across the top of the sterile barrier in response to movement of the hub adapter outside of the sterile field (as described in detail in FIG. 4). Movement of the hub adapter is driven by a drive system carried by the support table in which the guidewire hub 2909, the guide catheter hub 2914, the procedure catheter hub 2912, and the access catheter hub 2910 are installed upon.

The steps may further include driving a subset of the assembly in response to movement of each of the hub adapters along the support table until the subset of the assembly is positioned to perform a neurovascular procedure at a neurovascular treatment site. The subset of the assembly may include the guidewire 2907, the guide catheter 2906, and the procedure catheter 2904.

In some embodiments, the guidewire 2907, the guide catheter 2906 and the procedure catheter 2904 are advanced as a unit through (with respect to the guidewire 2907) and over (with respect to the guide catheter 2906 and the procedure catheter 2904) at least a portion of a length of the access (e.g., insert) catheter 2902 after supra-aortic access is achieved.

In some embodiments, the catheter assembly 2900 may be part of a robotic control system for achieving supra-aortic access and neurovascular treatment site access, as described in FIGS. 18A-18E. In some embodiments, the catheter assembly 2900 may be part of a manual control system for achieving supra-aortic access and neurovascular treatment site access. In some embodiments, the catheter assembly 2900 may be part of a hybrid control system (with manual and robotic components) for achieving supra-aortic access and neurovascular treatment site access. For example, in such hybrid systems, supra-aortic access may be robotically driven while neurovascular site access and embolectomy or other procedures may be manual. Alternatively, in such hybrid systems, supra-aortic access may be manual while neurovascular site access may be robotically achieved. Still further, in such hybrid systems, any one or more of: the guidewire, access catheter, guide catheter, or procedure catheter may be robotically driven or manually manipulated.

An example robotic control system may include at least a guidewire hub (e.g., guidewire hub 2909) configured to adjust each of an axial position and a rotational position of a guidewire 2907. The robotic control system may also include an access catheter hub 2910 configured to adjust axial and rotational movement of an access catheter 2902. The robotic control system may also include a guide catheter hub 2914 configured to control axial movement of a guide catheter 2906. The robotic control system may also include a procedure catheter hub 2912 configured to adjust an axial position and a rotational position of a procedure catheter 2904.

In some embodiments, the procedure catheter hub 2912 is further configured to laterally deflect a distal deflection zone of the procedure catheter 2904.

In some embodiments, the guidewire hub 2909 is configured to couple to a guidewire hub adapter by magnetically coupling the guidewire hub to a first drive magnet. The access catheter hub 2910 is configured to couple to an access catheter hub adapter by magnetically coupling the access catheter hub 2910 to a second drive magnet. The procedure catheter hub 2912 is configured to couple to a procedure catheter hub adapter by magnetically coupling the procedure catheter hub 2912 to a third drive magnet. The guide catheter hub 2914 is configured to couple to a guide catheter hub adapter by magnetically coupling the guide catheter hub 2914 to a fourth drive magnet. In some embodiments, the first drive magnet, the second drive magnet, the third drive magnet, and the fourth drive magnet are independently movably carried by a drive table.

In some embodiments, the robotic control system includes a first driven magnet on the guidewire hub 2909. The first driven magnet may be configured to cooperate with the first drive magnet such that the first driven magnet is configured to move in response to movement of the first drive magnet. In some embodiments, the first drive magnet is configured to move outside of a sterile field separated from the first driven magnet by a barrier while the first driven magnet is within the sterile field. In some embodiments, a position of the first driven magnet is movable in response to manipulation of a procedure drive control on a control console associated with the drive table. Drive magnets and driven magnet interactions are described in detail with respect to FIG. 4 above.

In some embodiments, the robotic control system includes a second driven magnet on the access catheter hub 2910. The second driven magnet may be configured to cooperate with the second drive magnet such that the second driven magnet is configured to move in response to movement of the second drive magnet. In some embodiments, the second drive magnet is configured to move outside of a sterile field separated from the second driven magnet by a barrier while the second driven magnet is within the sterile field.

In some embodiments, the robotic control system includes a third driven magnet on the procedure catheter hub 2912. The third driven magnet may be configured to cooperate with the third drive magnet such that the third driven magnet is configured to move in response to movement of the third drive magnet. In some embodiments, the third drive magnet is configured to move outside of a sterile field separated from the third driven magnet by a barrier while the third driven magnet is within the sterile field.

In some embodiments, the robotic control system includes a fourth driven magnet on the guide catheter hub 2914. The fourth driven magnet may be configured to cooperate with the fourth drive magnet such that the fourth driven magnet is configured to move in response to movement of the fourth drive magnet. In some embodiments, the fourth drive magnet is configured to move outside of a sterile field separated from the fourth driven magnet by a barrier while the fourth driven magnet is within the sterile field. In some embodiments, there may be more than four driven magnets and corresponding catheter hubs for control of additional catheters.

In some embodiments, devices (e.g., hubs, hub adapters, interventional devices, and/or trays) described herein may be used during a robotically driven procedure. For example, in a robotically driven procedure, one or more of the interventional devices may be driven through vasculature and to a procedure site. Robotically driving such devices may include engaging electromechanical components that are controlled by user input. In some implementations, users may provide the input at a control system that interfaces with one or more hubs and hub adapters.

In some embodiments, the hubs, hub adapters, interventional devices, and trays described herein may be used during a non-robotic (e.g., manually driven) procedure. Manually driving such devices may include engaging manually with the hubs to affect movement of the interventional devices.

In some embodiments, the devices described herein may be used to carry out a method of performing an intracranial procedure at an intracranial site. The method of performing the intracranial procedure may include any of the same steps as described herein for performing a neurovascular procedure. The procedure may be robotically performed, manually performed, or a hybridized combination of both.

While the foregoing describes magnetic coupling of hubs to drive magnets, in other embodiments, any of the interventional devices and/or hubs may be mechanically coupled to a drive system. Any of the methods described herein may include steps of mechanically coupling one or more interventional devices (e.g., the guidewire 2907, the access catheter 2902, the procedure catheter 2904, and/or the guide catheter 2906) and/or one or more hubs (e.g., the guidewire hub 2909, the access catheter hub 2910, the procedure catheter hub 2912, and/or the guide catheter hub 2914) with one or more drive mechanisms.

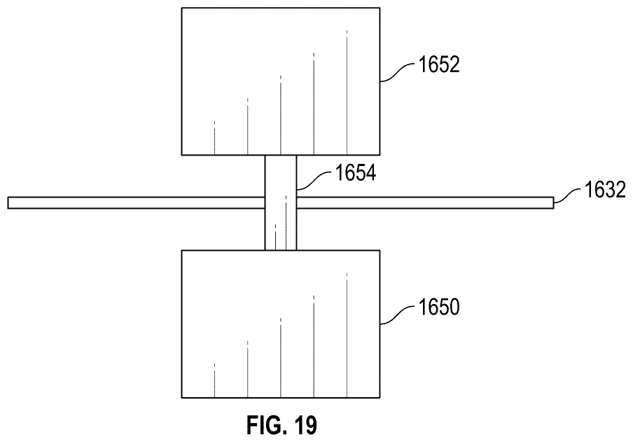

FIG. 19 illustrates a mechanical coupling mechanism 1654 between a drive mechanism 1650 and a driven mechanism 1652. Drive mechanism 1650 and driven mechanism 1652 may have any of the same or similar features or functions as the drive magnet 67 and driven magnet 69, respectively, except as otherwise described herein. The drive mechanism 1650 may be part of or coupled to a hub adapter (e.g., the hub adapter 48). The driven mechanism 1652 may be part of or coupled to a hub (e.g., the hub 36, the guidewire hub 2909, the access catheter hub 2910, the procedure catheter hub 2912, or the guide catheter hub 2914). In some instances, the mechanical coupling mechanism 1654 may comprise a structural support (e.g., a support rod or support strut) extending transversely through a seal in a sterile barrier 1632. The seal may permit the structural support to be advanced along a length of the sterile barrier 1632, while still maintaining a seal with the structural support to maintain the sterile field, as the drive mechanism 1650 and driven mechanism 1652 are advanced and/or retracted as described herein. For example, the seal may comprise a tongue and groove closure mechanism along the sterile barrier 1632 that is configured to close on either side of the structural support while permitting passage of the structural support through the sterile barrier 1632 and maintaining a seal against the structural support as the structural support is advanced along the length of the sterile barrier 1632.

In some embodiments, the structural support can extend through an elongate self closing seal between two adjacent coaptive edges of flexible material (e.g., similar in shape to a duckbill valve) that extends along an axis. As the structural support advances along the axis between the coaptive edges, the coaptive edges may permit the structural support to advance, and then may be biased back into a sealing engagement with each other as the structural support passes any given point along the axis.

In some embodiments, the drive mechanism may be a splined drive shaft (e.g., a non-sterile splined drive shaft). The mechanical coupling 1654 can include a pulley within a plate that serves as the sterile barrier 1632 and a sterile splined shaft configured to couple to the driven mechanism 1652. The driven mechanism 1652 can be a sterile pulley that receives the sterile splined shaft from the sterile barrier. In some embodiments, one or more splined drive shafts can engage and turn corresponding pulleys in the plate that serves as the sterile barrier. Each hub can have a sterile pulley that is configured to receive a sterile splined shaft from the sterile barrier plate. Rotation of the splined drive shaft can turn the pulley in the sterile barrier plate which can in turn turn the sterile pulley in the hub via the sterile splined shaft.

It will be understood by one having skill in the art that any embodiment as described herein may be modified to incorporate a mechanical coupling mechanism, for example, as shown in FIG. 19.

The interventional devices described herein may be provided individually or at least some of the interventional devices can be provided in a preassembled (e.g., nested or stacked) configuration. For example, the interventional devices may be provided in the form of an interventional device assembly, such as interventional device assembly 2900, in a concentric nested or stacked configuration. If provided individually, each catheter (and in some embodiments, each corresponding catheter hub) can be unpackaged and primed to remove air from its inner lumen, for example, by flushing the catheter (and in some embodiments, the corresponding catheter hub) to remove air by displacing it with a fluid, such as saline, contrast media, or a mixture of saline and contrast media. After priming, the interventional devices can be manually assembled into a stacked configuration so that they are ready for introduction into the body for a surgical procedure, for example, via an introducer sheath.

Assembling the devices into a stacked configuration can include individually inserting interventional devices into one another by order of size. For example, an interventional device having a second largest diameter can be inserted into the lumen of an interventional device having a largest diameter. An interventional device having a third largest diameter can then be inserted into the interventional device having the second largest diameter and so on.

For example, with respect to FIG. 17, assembly can be performed by first inserting a distal end of the catheter 2904 through the hub 2914 and into the catheter 2906. The catheter 2904 can be advanced through the catheter 2906 until the distal tip of the catheter 2904 is flush with or extends beyond the distal tip of the catheter 2906, and/or until the catheter 2904 cannot be inserted any further. Then, the distal end of the catheter 2902 can be inserted through the hub 2912 and into the catheter 2904. The catheter 2902 can be advanced through the catheter 2904 until the distal tip of the catheter 2902 is flush with or extends beyond the distal tip of the catheter 2904, and/or until the catheter 2902 cannot be inserted any further. Then, the distal end of the guidewire 2907 can be inserted through the hub 2910 and into the catheter 2902. The guidewire 2907 can be advanced through the catheter 2902 until the distal tip of the guidewire 2907 is flush with or extends beyond the distal tip of the catheter 2902, and/or until the guidewire 2907 cannot be inserted any further.

Embodiments in which two or more of the interventional devices are packaged together as a single unit in an assembled (e.g., nested or stacked) configuration may provide efficient unpackaging and preparation prior to use and efficient assembly within a robotic control system. The interventional devices may be pre-mounted to their respective hubs prior to packaging. In certain embodiments, two or three or more interventional devices may be packaged in a fully nested (i.e., fully axially inserted) configuration or nearly fully nested configuration. In a fully nested configuration, each interventional device is inserted as far as possible into an adjacent distal hub and interventional device. Such a fully nested configuration may minimize a total length of the interventional device assembly and minimize the size of the packaging required to house the interventional device assembly.

In some embodiments, the interventional devices may also be sterilized prior to packaging while in the assembled configuration, for example, using ethylene oxide gas. In some embodiments, the interventional devices may be packaged while in the assembled configuration before sterilization with ethylene oxide gas. For interventional devices in a nested or stacked configuration, ethylene oxide gas can be provided in a space between adjacent interventional devices (for example, an annular lumen between an outer diameter of a first interventional device nested within a second interventional device and the inner diameter of the second interventional device) for sterilization. In some embodiments, the interventional device assembly can be packaged in a thermoformed tray and sealed with an HDPE (e.g., Tyvek®) lid. The interventional device assembly can be unpackaged by removal (e.g., opening or peeling off) of the lid by a user in a non-sterile field. A user in the sterile field can then remove the interventional device assembly and place it on the sterile work surface, for example, of a robotic drive table, as described herein.

Packaging the interventional devices in an assembled configuration and sterilized state can reduce the time associated with unpackaging and assembly of individual interventional devices and facilitate efficient connection to a robotic drive system. Each interventional device and hub combination may further be packaged with a fluidics connection for coupling to a fluid source and/or a vacuum source. In some embodiments, each hub or a hemostasis valve coupled to the hub may include the fluidics connection.

After the interventional device assembly is unpackaged (e.g., after the interventional device assembly is positioned on the robotic drive table), priming can be performed while the devices are concentrically nested or stacked. This is preferably accomplished in each fluid lumen, such as, for example, the annular lumen between the catheter 2906 and the catheter 2904 and in between each of the additional concentric interventional devices in the concentric stack. In certain embodiments, the fluid lumen can include a lumen between a distal hub and a proximal interventional device, such as, for example, the lumen between the hub 2914 and the catheter 2904. In certain embodiments, priming can be performed while the devices are still in the sterile packaging.

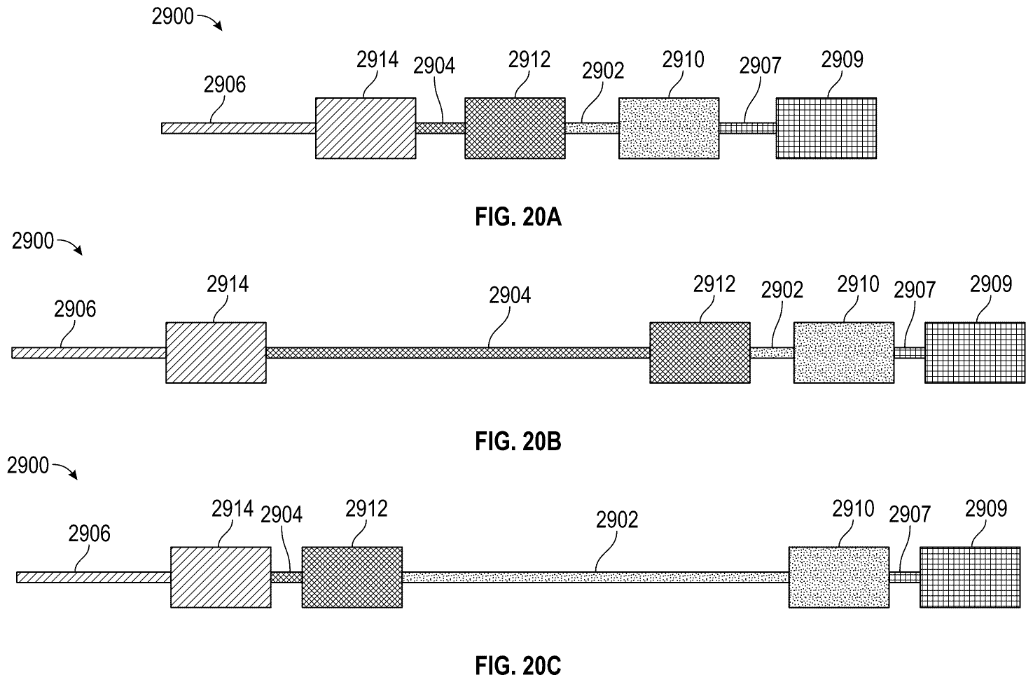

The fluidics connections can be connected to a fluidics system for delivering saline and contrast media to the catheters and providing aspiration. In some embodiments, the fluidics connections may be passed outside the sterile field for connection to the fluidics system. Once connected, the fluidics system can perform a priming sequence to flush each catheter of the interventional device assembly with fluid (e.g., saline, contrast media, or a mixture of saline and contrast media). The priming sequence may also include flushing each corresponding catheter hub with fluid. The fluid may be de-aired or de-gassed by the fluidics system prior to priming. In some embodiments, a vacuum source of the fluidics system can also be used to evacuate air from each catheter while flushing with fluid. In certain embodiments, a tip of the catheter can be placed into a container of fluid, such as saline, contrast media, or a mixture of saline and contrast media, during priming so that the fluid in the container, and not air, is aspirated through the tip of the catheter when the vacuum source is applied. In other embodiments, the tip of the catheter may be blocked (for example, using a plug) so that air is not aspirated from the tip of the catheter when the vacuum source is applied. In certain embodiments, the priming process may be automated such that a user can provide a single command and each catheter (and in some embodiments, each corresponding catheter hub) can be primed, sequentially (for example, as described with respect to FIGS. 20A-20C) or simultaneously.

Additional details regarding fluidics systems are disclosed in U.S. patent application Ser. No. 17/879,614, entitled Multi Catheter System With Integrated Fluidics Management, filed Aug. 2, 2022, which is hereby expressly incorporated in its entirety herein.

Fluid resistance within a lumen may be greater when there is a reduction in cross sectional luminal area for flow, for example, when a second interventional device (e.g., a catheter or guidewire) extends within the lumen of a first interventional device. The amount of fluid resistance can be affected by the length of the cross sectional narrowing, for example, due to a depth of axial insertion of the second interventional device within the first interventional device. A second interventional device extending partially through the lumen of a first interventional device will provide a smaller length of cross-sectional narrowing, and accordingly may result in a lower fluid resistance within the lumen of the first catheter, than if the second interventional device were to extend entirely through the lumen of the first interventional device. Thus, fluid resistance can be lowered by at least partially decreasing a depth of axial insertion (i.e., axial overlap) of a second interventional device into the lumen through which fluid is to be injected (e.g., a length of the second interventional device into its concentrically adjacent lumen).

In some embodiments, over certain depths of insertion of a second interventional device within a first interventional device (for example, when the second interventional device is at or near a maximum insertion depth within the first interventional device), the size of the fluid channel between the devices (e.g., the annular lumen between the first interventional device and the second interventional device) can lead to higher than desirable amounts of fluid resistance during a priming procedure. In some embodiments, the depth of insertion of the second interventional device within the first interventional device can be decreased to reduce the pressure needed to prime the catheter and reduce internal interference.

In some embodiments, a catheter in the interventional device assembly can be separated from the other interventional devices for priming to reduce the pressure needed to prime the catheter and reduce internal interference. The catheter being primed may be separated from the interventional devices within the lumen of the catheter by proximally retracting the interventional devices within the lumen of the catheter. For example, the interventional devices within the lumen of the catheter being primed can be proximally retracted from the catheter being primed as far as possible while still maintaining a nested or stacked relationship (e.g., at least about 2 cm or 5 cm or more axial overlap) in order to minimize the pressure needed to prime the catheter and minimize internal interference. In other words, a catheter can be separated from more proximal interventional devices for priming while a distal tip of an adjacent proximal interventional device is still positioned within the lumen of the catheter. Maintaining at least some of the distal tip of an adjacent proximal interventional device within the lumen of the catheter may allow for easier reinsertion and advancement of the proximal interventional device after priming.

In some embodiments, the axial overlap may be between about 2 cm and about 20 cm, between about 2 cm and 10 cm, between about 2 cm and 5 cm, between about 5 cm and 20 cm, between about 5 cm and 10 cm, or any other suitable range. In some embodiments, the axial overlap may be at least about 2 cm, at least about 5 cm, at least about 10 cm, at least about 20 cm, no more than 2 cm, no more than 5 cm, no more than 10 cm, no more than 20 cm, about 2 cm, about 5 cm, about 10 cm, about 20 cm, or any other suitable amount.

In some embodiments, the robotic drive table can be programed to proximally retract the inner interventional device(s) from the catheter being primed as much as possible while still maintaining a nested or stacked relationship. In other embodiments, the robotic drive table can be programmed to separate inner devices from the catheter being primed to a distance sufficient to optimize the length of the unobstructed lumen and result in an amount of fluid resistance lower than a threshold value. After the catheter being primed is separated from the other interventional devices, the catheter can be primed by flushing the catheter with fluid, such as saline, contrast media, or a mixture of saline and contrast media.

After the catheter is primed, it may be returned to an initial position and a next catheter of the interventional device assembly can be separated from the other interventional devices within its lumen for priming. This sequence can be repeated for each catheter of the interventional device assembly. In other embodiments, after a catheter is primed, it may be advanced to a ready or drive position to begin insertion into the patient. While the foregoing describes separating catheters to be primed by retraction of inner interventional devices, an outer catheter may also be separated from inner interventional devices by distally axially advancing the outer catheter relative to the inner interventional devices. An example of a priming process is described with respect to FIGS. 20A-20C.

FIG. 20A depicts the interventional device assembly 2900 assembled in a concentric stack and axially compressed configuration. As shown in FIG. 20A, the interventional devices can be fully nested within each other. This may be the configuration following unpackaging of the device assembly 2900 and placement onto the robotic drive table. A priming sequence may begin by distally axially advancing the catheter 2906 and hub 2914 relative to the catheter 2904, hub 2912, catheter 2902, hub 2910, guidewire 2907, and hub 2909, for example, as far as possible while maintaining a distal tip of the catheter 2904 within the lumen of the catheter 2906, as shown in FIG. 20B, or to a distance that will result in a desirable amount of fluid resistance for priming. In some embodiments, the catheter 2906 is advanced in response to a control signal from a control system. The catheter 2906 can then be primed by introducing priming fluid using the fluidics system. In some embodiments, priming fluid is introduced in response to a control signal from a control system. Priming the catheter 2906 can include priming the hub 2914. For example, in certain embodiments, the hub 2914 or a hemostasis valve coupled thereto can include fluidics connections to receive priming fluid from the fluidics system. After priming, the catheter 2906 can be returned to its initial position (e.g., the fully axially compressed configuration) as shown in FIG. 20A. In some embodiments, the catheter 2906 is returned to its initial position in response to a control signal from a control system.

After the catheter 2906 is primed and returned to its initial position, the catheter 2904 and hub 2912 can be distally axially advanced relative to the catheter 2902, hub 2910, guidewire 2907 and hub 2909 (also distally axially advancing the catheter 2906 and hub 2914 without changing or minimally changing their relative position with respect to catheter 2904), for example, as far as possible while maintaining a distal tip of the catheter 2902 within the lumen of the catheter 2904, as shown in FIG. 20C, or to a distance that will result in a desirable amount of fluid resistance for priming. In some embodiments, the catheter 2904 and the catheter 2906 are advanced in response to a control signal from a control system. The catheter 2904 can then be primed by introducing priming fluid using the fluidics system. In some embodiments, priming fluid is introduced in response to a control signal from a control system. Priming the catheter 2904 can include priming the hub 2912. For example, in certain embodiments, the hub 2912 or a hemostasis valve coupled thereto can include fluidics connections to receive priming fluid from the fluidics system. After priming, the catheter 2904 and catheter 2906 can be returned to their initial positions (e.g., the fully axially compressed configuration) as shown in FIG. 20A. In some embodiments, the catheter 2904 and the catheter 2906 are returned to their initial position in response to a control signal from a control system.

After the catheter 2904 is primed and returned to its initial position, the catheter 2902 and hub 2910 can be distally axially advanced relative to the guidewire 2907 and hub 2909 (also distally axially advancing the catheter 2906, hub 2914, catheter 2904, and hub 2912 without changing or minimally changing their relative positions with respect to the catheter 2902), for example, as far as possible while maintaining a distal tip of the guidewire 2907 within the lumen of the catheter 2902, or to a distance that will result in a desirable amount of fluid resistance for priming. In some embodiments, the catheter 2902, the catheter 2904, and the catheter 2906 are advanced in response to a control signal from a control system. The catheter 2902 can then be primed by introducing priming fluid using the fluidics system. In some embodiments, priming fluid is introduced in response to a control signal from a control system. Priming the catheter 2902 can include priming the hub 2910. For example, in certain embodiments, the hub 2910 or a hemostasis valve coupled thereto can include fluidics connections to receive priming fluid from the fluidics system. After priming, the catheter 2902 and catheters 2904 and 2906 can be returned to their initial positions (e.g., the fully axially compressed configuration) shown in FIG. 20A. In some embodiments, the catheter 2902, the catheter 2904, and the catheter 2906 are returned to their initial position in response to a control signal from a control system.

In some embodiments, the priming procedure described with respect to FIGS. 20A-20C may be performed in response to a single control signal from a control system. In other embodiments, various steps of the priming procedure may be performed in response to unique control signals. In some embodiments, priming of each unique interventional device can be performed in response to a unique control signal.

In alternative embodiments, each of the catheters can be distally separated from one another simultaneously for priming. For example, the catheter 2902 can be distally separated from the guidewire 2907 while maintaining the distal tip of the guidewire 2907 in the lumen of the catheter 2902, the catheter 2904 can be distally separated from the catheter 2902 while maintaining the distal tip of the catheter 2902 in the lumen of the catheter 2904, and the catheter 2906 can be distally separated from the catheter 2904 while maintaining the distal tip of the catheter 2904 in the lumen of the catheter 2906 simultaneously. However, an embodiment in which only one set of adjacent hubs is separated at a time, as described with respect to FIGS. 20A-20C, can provide a smaller overall length of the assembly at any particular time, which can allow for use with a smaller robotic drive system. While separation of outer catheters from their inner interventional devices is described as distally axially advancing the catheters relative to their inner interventional devices, separation can include proximally retracting the inner interventional devices from the outer catheters.

In alternative embodiments, one or more of the catheter 2902, the catheter 2904, and the catheter 2906 can be advanced to a ready or drive position to begin insertion into the patient after priming (e.g., prior to priming a subsequent catheter). In such embodiments, the catheters may advance to the ready or drive position without returning to their initial position after priming.

As described above, in some embodiments, the catheters 2902, 2904, and 2906 may be assembled into the concentric stack orientation illustrated in FIG. 17 prior to flushing the catheters to remove air by displacing it with a fluid such as saline contrast media, or a mixture of saline and contrast media. This is preferably accomplished in each fluid lumen, such as, for example, the annular lumen between the catheter 2906 and the catheter 2904 and in between each of the additional concentric interventional devices in the concentric stack. Infusing fluid (e.g., saline, contrast media, or a mixture of saline and contrast media) under pressure may displace substantially all of the air but some small bubbles may remain, adhering to the inside wall of an outer catheter (e.g., the guide catheter 2906), the outside wall of an inner catheter (e.g., the procedure catheter 2904), or both.

While fluid is being introduced under pressure into the proximal end of the annular lumen (e.g., into a hub of the outer catheter or a hemostasis valve coupled thereto), the inner catheter may be moved with respect to the outer catheter, to disrupt the holding forces between the microbubbles and adjacent wall and allow the bubbles to be carried downstream and out through the distal opening of the lumen or removed via aspiration. The catheters may be moved axially, rotationally or both with respect to each other. In certain embodiments, the catheters may be reciprocated axially, rotationally, or both with respect to each other. In some embodiments, the catheters may be moved intermittently axially, rotationally, or both. In other embodiments, the catheters may be rotated continuously or in a constant direction.

In some implementations, a first catheter is moved reciprocally with respect to an adjacent catheter or guidewire such as axially over a stroke length in a range of from about 1 mm to about 250 mm, from about 10 mm to about 250 mm, from about 5 mm to about 125 mm, from about 25 mm to about 125 mm, from about 10 mm to about 50 mm, from about 15 mm to about 30 mm, from about 5 mm to about 30 mm, from about 15 mm to about 25 mm, from about 20 mm to about 40 mm, or any other suitable range. In some implementations, a first catheter is moved reciprocally with respect to an adjacent catheter or guidewire such as axially over a stroke length of at least 5 mm, at least 10 mm, at least 15 mm, at least 20 mm, at least 25 mm, at least 30 mm, at least 50 mm, no more than 10 mm, no more than 20 mm, no more than 25 mm, no more than 30 mm, no more than 50 mm, no more than 125 mm, no more than 150 mm, about 5 mm, about 10 mm, about 15 mm, about 20 mm, about 25 mm, about 30 mm, about 50 mm, or any other suitable stroke length.

In some implementations, a first catheter is moved reciprocally with respect to an adjacent catheter or guidewire such as axially at a reciprocation frequency in a range of from about 0.5 Hz to about 1 Hz, from about 1 Hz to about 5 Hz, from about 1 Hz to about 10 Hz, from about 1 Hz to about 25 Hz, from about 5 Hz to about 10 Hz, from about 10 Hz to about 25 Hz, or any other suitable range of frequencies. In some implementations, the first catheter is moved reciprocally with respect to an adjacent catheter or guidewire such as axially at a reciprocation frequency of at least 0.5 Hz, at least 1 Hz, at least 2 Hz, at least 5 Hz, at least 10 Hz, at least 25 Hz, no more than 0.5 Hz, no more than 1 Hz, no more than 2 Hz, no more than 5 Hz, no more than 10 Hz, no more than 25 Hz, about 0.5 Hz, about 1 Hz, about 2 Hz, about 5 Hz, about 10 Hz, about 25 Hz or any other suitable frequency.

In one implementation, a first catheter is moved reciprocally with respect to the adjacent catheter or guidewire such as axially over a stroke length in a range of from about 0.5 inches to about 10 inches, or from about one inch to about 5 inches at a reciprocation frequency of no more than about 5 cycles per second or two cycles per second or less.

In some implementations, a first catheter is moved reciprocally with respect to an adjacent catheter or guidewire such as rotationally over an angle of rotation per stroke in a range of from about 5 degrees to about 180 degrees, from about 5 degrees to about 360 degrees, from about 15 degrees to about 180 degrees, from about 15 degrees to about 150 degrees, from about 15 degrees to about 120 degrees, from about 15 degrees to about 90 degrees, form about 15 degrees to about 60 degrees, from about 15 degrees to about 30 degrees, from about 30 degrees to about 180 degrees, from about 30 degrees to about 150 degrees, from about 30 degrees to about 120 degrees, from about 30 degrees to about 90 degrees, form about 30 degrees to about 60 degrees, from about 60 degrees to about 180 degrees, from about 60 degrees to about 150 degrees, from about 60 degrees to about 120 degrees, from about 60 degrees to about 90 degrees, from about 90 degrees to about 180 degrees, from about 90 degrees to about 150 degrees, from about 90 degrees to about 120 degrees, from about 120 degrees to about 180 degrees, from about 120 degrees to about 150 degrees, from about 150 degrees to about 180 degrees or any other suitable range. In some implementations, a first catheter is moved reciprocally with respect to an adjacent catheter or guidewire such as rotationally over an angle of rotation per stroke of at least 5 degrees, at least 15 degrees, at least 30 degrees, at least 60 degrees, at least 90 degrees, at least 120 degrees, at least 150 degrees, at least 180 degrees, at least 360 degrees, no more than 5 degrees, no more than 15 degrees, no more than 30 degrees, no more than 60 degrees, no more than 90 degrees, no more than 120 degrees, no more than 150 degrees, no more than 180 degrees, no more than 360 degrees, about 5 degrees, about 15 degrees, about 30 degrees, about 60 degrees, about 90 degrees, about 120 degrees, about 150 degrees, about 180 degrees, about 360 degrees, or any other suitable angle.

In some implementations, a first catheter is moved reciprocally with respect to an adjacent catheter or guidewire such as rotationally at a reciprocation frequency in a range of from about 0.5 Hz to about 1 Hz, from about 1 Hz to about 5 Hz, from about 1 Hz to about 10 Hz, from about 1 Hz to about 25 Hz, from about 5 Hz to about 10 Hz, from about 10 Hz to about 25 Hz, or any other suitable range of frequencies. In some implementations, the first catheter is moved reciprocally with respect to an adjacent catheter or guidewire such as rotationally at a reciprocation frequency of at least 0.5 Hz, at least 1 Hz, at least 2 Hz, at least 5 Hz, at least 10 Hz, at least 25 Hz, no more than 0.5 Hz, no more than 1 Hz, no more than 2 Hz, no more than 5 Hz, no more than 10 Hz, no more than 25 Hz, about 0.5 Hz, about 1 Hz, about 2 Hz, about 5 Hz, about 10 Hz, about 25 Hz or any other suitable frequency.

In some implementations, a first catheter is moved reciprocally with respect to an adjacent catheter or guidewire for a number of reciprocations between 1 and 200, between 1 and 100, between 1 and 50, between 1 and 25, between 1 and 15, between 1 and 10, between 1 and 5, between 5 and 25, between 5 and 15, between 5 and 10, or any other suitable range. In some implementations, a first catheter is moved reciprocally with respect to an adjacent catheter or guidewire for at least 1 reciprocation, at least 2 reciprocations, at least 5 reciprocations, at least 10 reciprocations, at least 15 reciprocations, at least 25 reciprocations, at least 50 reciprocations, no more than 5 reciprocations, no more than 10 reciprocations, no more than 15 reciprocations, no more than 25 reciprocations, no more 50 than reciprocations, no more than 100 reciprocations, no more than 200 reciprocations, about 1 reciprocation, about 2 reciprocations, about 5 reciprocations, about 10 reciprocations, about 25 reciprocations, about 50 reciprocations, about 100 reciprocations, about 200 reciprocations, or any other suitable number. One reciprocation can include a movement (axially or rotationally) from a first position to a second position followed by a return from the second position to the first position.

In some implementations, a first catheter is moved reciprocally with respect to an adjacent catheter or guidewire over a length of time in a range of from 1 about second to about 60 seconds, from about 1 second to about 45 seconds, from about 1 second to about 30 seconds, from about 1 second to about 20 seconds, from about 1 second to about 15 seconds, from about 1 second to about 10 seconds, from about 5 seconds to about 45 seconds, from about 5 seconds to about 30 seconds, from about 5 seconds to about 20 seconds, from about 5 seconds to about 15 seconds, from about 5 seconds to about 10 seconds, from about 10 seconds to about 30 seconds, form about 10 seconds to about 20 seconds, or any other suitable range. In some implementations, a first catheter is moved reciprocally with respect to an adjacent catheter or guidewire over a length of time of at least 1 second, at least 5 seconds, at least 10 seconds, at least 15 seconds, at least 20 seconds, at least 30 seconds, at least 45 seconds, at least 60 seconds, no more than 5 seconds, no more than 10 seconds, no more than 15 seconds, no more than 20 seconds, no more than 30 seconds, no more than 45 seconds, no more than 60 seconds, about 5 seconds, about 10 seconds, about 15 seconds, about 20 seconds, about 30 seconds, about 45 seconds, about 60 seconds, or any other suitable length of time.

Reciprocation of adjacent catheters to disrupt microbubbles may be accomplished manually by grasping the corresponding catheter hubs and manually moving the catheters axially or rotationally with respect to each other while delivering pressurized fluid (e.g., saline, contrast media, or a mixture of saline and contrast media). Alternatively, such as in a robotically driven system, a processor may be configured to robotically drive at least one of two adjacent catheter hubs (for example, at least one of hub 2914 and hub 2912) to achieve relative movement between the adjacent catheters thereby disrupting and expelling microbubbles, such as in response to user activation of a flush control. For example, in certain embodiments, two adjacent interventional devices may be moved relative to one another in response to a control signal from a control system. In certain embodiments, delivery of pressurized fluid may be performed in response to a control signal from a control system.

The reciprocation of adjacent catheters may generate shear forces that dislodge the air bubbles. For example, relative movement of the inner and outer surfaces of adjacent catheters may increase the fluid shear rate between the adjacent catheters during priming in comparison to static surfaces. In some embodiments, the shear force can be increased by increasing the flow rate of the solution (e.g., saline, contrast media, or a mixture of saline and contrast media) being provided by the fluidics system. In certain embodiments, both flow rate and relative movement between adjacent catheters are controlled to dislodge air bubbles.

In some embodiments, after each catheter is primed by the fluidics system, an ultrasound bubble detector may be used to confirm that the catheters are substantially free of air bubbles. For example, an ultrasound chip (such as mounted within a hub adjacent a catheter receiving lumen) may be run along the length of the catheters to confirm that no air bubbles remain in the system.

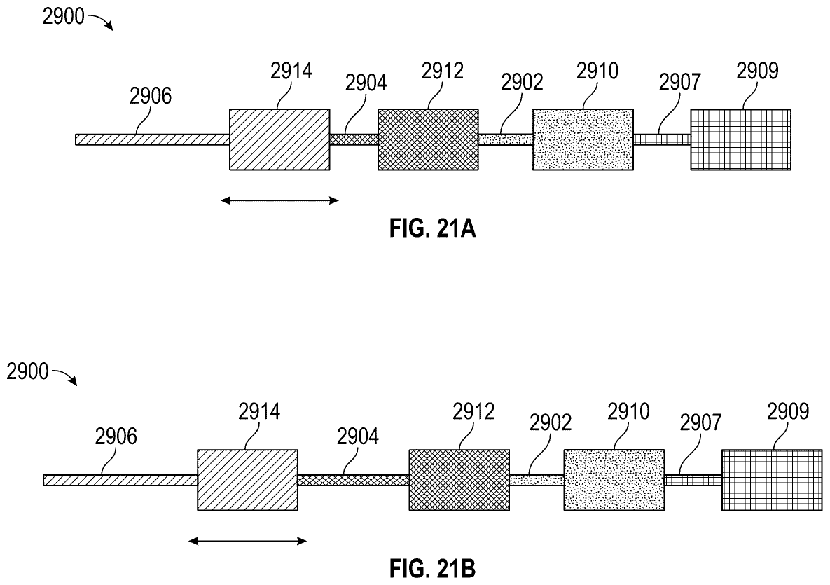

An example of a priming process including reciprocal movement of adjacent catheters is described with respect to FIGS. 21A-21B.

FIG. 21A depicts the interventional device assembly 2900 assembled in a concentric stack configuration. As shown in FIG. 21A, the interventional devices can be fully nested within each other. This may be the configuration following unpackaging of the device assembly 2900 and placement onto the robotic drive table. Alternatively, individual interventional devices of the device assembly 2900 can be assembled into the device assembly 2900 on the drive table.

A priming sequence may begin by priming the catheter 2906. In some embodiments, the catheter 2906 can be primed by introducing fluid (e.g., saline, contrast media, or a mixture of saline and contrast media) under pressure into the lumen of the catheter 2906 while generating reciprocal movement of catheter 2906 and/or hub 2914, axially, rotationally or both, relative to the catheter 2904. Priming the catheter 2906 can include priming the hub 2914. For example, in certain embodiments, the hub 2914 or a hemostasis valve coupled thereto can include fluidics connections to receive priming fluid from the fluidics system. In certain embodiments, the catheter 2906 and/or hub 2914 can be axially agitated back and forth along a longitudinal axis of the catheter 2906 (e.g., between the position of FIG. 21A and the position of FIG. 21B). Axial and/or rotational reciprocal motion of the catheter 2906 and/or hub 2914 can be performed manually or by a robotic drive table. Reciprocal movement may be generated in response to a control signal from a control system. Introducing fluid under pressure may be performed in response to a control signal from a control system.

In some embodiments, priming of the catheter 2906 may be performed by introducing fluid (e.g., saline, contrast media, or a mixture of saline and contrast media) under pressure into the lumen of the catheter 2906 while generating reciprocal movement of the catheter 2904 and/or hub 2912, axially, rotationally or both, relative to the catheter 2906. Axial and/or rotational reciprocal motion of the catheter 2904 and/or hub 2912 can be performed manually or by a robotic drive table. Reciprocal movement may be generated in response to a control signal from a control system. Introducing fluid under pressure may be performed in response to a control signal from a control system.

In some embodiments, priming of the catheter 2906 may be performed by introducing fluid (e.g., saline, contrast media, or a mixture of saline and contrast media) under pressure into the lumen of the catheter 2906 while generating reciprocal movement of both the catheter 2906 (and/or hub 2914) and the catheter 2904 (and/or hub 2912), axially, rotationally or both, relative to one another. Reciprocal movement may be generated in response to a control signal from a control system. Introducing fluid under pressure may be performed in response to a control signal from a control system.

In some embodiments, after priming the catheter 2906, the catheter 2906 can be returned to an initial position as shown in FIG. 21A. In other embodiments, after priming the catheter 2906, the catheter 2906 can be advanced to a ready or drive position to begin insertion into the patient.

In some embodiments, after the catheter 2906 is primed, the catheter 2904 can be primed. Priming the catheter 2904 can include priming the hub 2912. For example, in certain embodiments, the hub 2912 or a hemostasis valve coupled thereto can include fluidics connections to receive priming fluid from the fluidics system. In some embodiments, the catheter 2904 can be primed by introducing fluid (e.g., saline, contrast media, or a mixture of saline and contrast media) under pressure into the lumen of the catheter 2904 while generating reciprocal movement of the catheter 2904 and/or hub 2912, axially, rotationally or both, relative to the catheter 2902. Reciprocal movement may be generated in response to a control signal from a control system. Introducing fluid under pressure may be performed in response to a control signal from a control system.

In some embodiments, priming of the catheter 2904 may be performed by introducing fluid (e.g., saline, contrast media, or a mixture of saline and contrast media) under pressure into the lumen of the catheter 2904 while generating reciprocal movement of the catheter 2902 and/or hub 2910, axially, rotationally or both, relative to the catheter 2904. Axial and/or rotational reciprocal motion of the catheter 2902 and/or hub 2910 can be performed manually or by a robotic drive table. Reciprocal movement may be generated in response to a control signal from a control system. Introducing fluid under pressure may be performed in response to a control signal from a control system.

In some embodiments, priming of the catheter 2904 may be performed by introducing fluid (e.g., saline, contrast media, or a mixture of saline and contrast media) under pressure into the lumen of the catheter 2904 while generating reciprocal movement of both the catheter 2904 (and/or hub 2912) and the catheter 2902 (and/or hub 2910), axially, rotationally or both, relative to one another. Reciprocal movement may be generated in response to a control signal from a control system. Introducing fluid under pressure may be performed in response to a control signal from a control system.

In some embodiments, after priming the catheter 2904, the catheter 2904 can be returned to an initial position as shown in FIG. 21A. In some embodiments, after priming the catheter 2904, the catheter 2904 can be advanced to a ready or drive position to begin insertion into the patient.

In some embodiments, after the catheter 2904 is primed, the catheter 2902 can be primed. Priming the catheter 2902 can include priming the hub 2910. For example, in certain embodiments, the hub 2910 or a hemostasis valve coupled thereto can include fluidics connections to receive priming fluid from the fluidics system. In some embodiments, the catheter 2902 can be primed by introducing fluid (e.g., saline, contrast media, or a mixture of saline and contrast media) under pressure into the lumen of the catheter 2902 while generating reciprocal movement of the catheter 2902 and/or hub 2910, axially, rotationally or both, relative to the guidewire 2907. Reciprocal movement may be generated in response to a control signal from a control system. Introducing fluid under pressure may be performed in response to a control signal from a control system.

In some embodiments, priming of the catheter 2902 may be performed by introducing fluid (e.g., saline, contrast media, or a mixture of saline and contrast media) under pressure into the lumen of the catheter 2902 while generating reciprocal movement of the guidewire 2907 and/or hub 2909, axially, rotationally or both, relative to the catheter 2902. Axial and/or rotational reciprocal motion of the guidewire 2907 and/or hub 2909 can be performed manually or by a robotic drive table. Reciprocal movement may be generated in response to a control signal from a control system. Introducing fluid under pressure may be performed in response to a control signal from a control system.

In some embodiments, priming of the catheter 2902 may be performed by introducing fluid (e.g., saline, contrast media, or a mixture of saline and contrast media) under pressure into the lumen of the catheter 2902 while generating reciprocal movement of both the catheter 2902 (and/or hub 2910) and the guidewire 2907 (and/or hub 2909), axially, rotationally or both, relative to one another. Reciprocal movement may be generated in response to a control signal from a control system. Introducing fluid under pressure may be performed in response to a control signal from a control system.

In some embodiments, after priming the catheter 2902, the catheter 2902 can be returned to an initial position as shown in FIG. 21A. In other embodiments, after priming the catheter 2902, the catheter 2902 can be advanced to a ready or drive position to begin insertion into the patient.

In some embodiments, the priming procedure described with respect to FIGS. 21A and 21B may be performed in response to a single control signal from a control system. In other embodiments, various steps of the priming procedure may be performed in response to unique control signals. In some embodiments, priming of each unique interventional device can be performed in response to a unique control signal.

In the priming sequence described herein with respect to FIGS. 21A and 21B, the catheters are primed in order starting with the catheter 2906, followed by the catheter 2904, and then followed by the catheter 2902. However, it is contemplated that the catheters may be primed in any order. The catheters may be primed in series as described above with respect to FIGS. 21A and 21B. Alternatively, two or more of the catheters or each of the catheters may be primed in parallel.

In certain embodiments, priming the catheters can include decreasing a depth of axial insertion (i.e., axial overlap) of a second interventional device into the lumen of a first interventional device through which fluid is to be injected (e.g., a length of the second interventional device into its concentrically adjacent lumen), as described with respect. to FIGS. 20A-20C, and also generating relative reciprocal movement, axially, rotationally or both, between first interventional device and the second interventional device during priming, as discussed with respect to FIGS. 21A and 21B.

In some implementations, priming of a catheter can include vibrating at least a portion of the catheter and/or its associated hub when included. Vibration can be induced, for example, by an electric motor incorporated into a hub of the catheter, or by a separate electric motor or source of vibration put against the catheter when priming. In some implementations, at least a portion of the support table on which the catheters and/or their associated hubs are placed upon can vibrate during priming of any one or more catheters to aid in removal of air and/or microbubbles of air. Such vibration can be performed by an electric motor.

### EXAMPLES

Additional embodiments are disclosed in further detail in the following examples, which are not in any way intended to limit the scope of the claims.

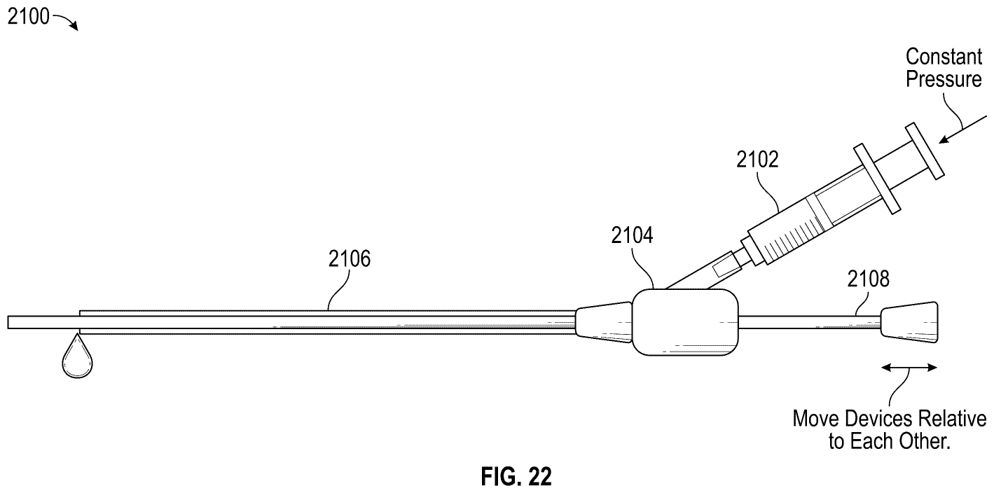

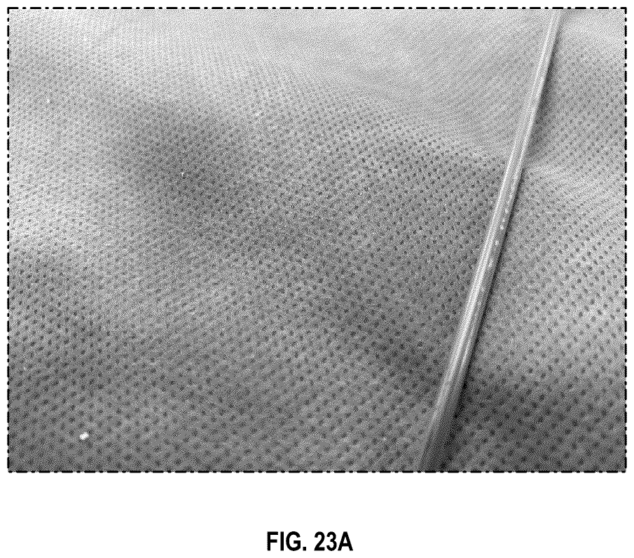

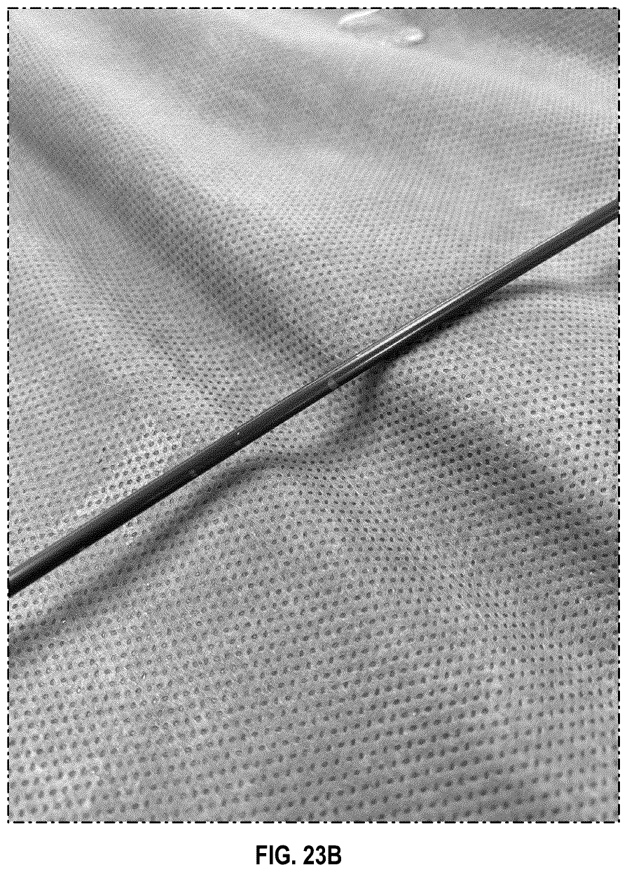

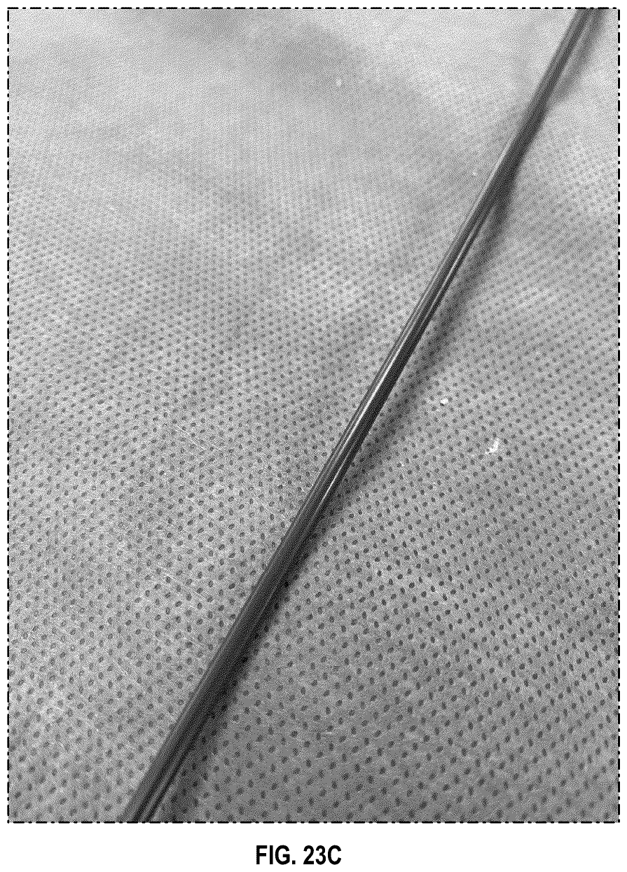

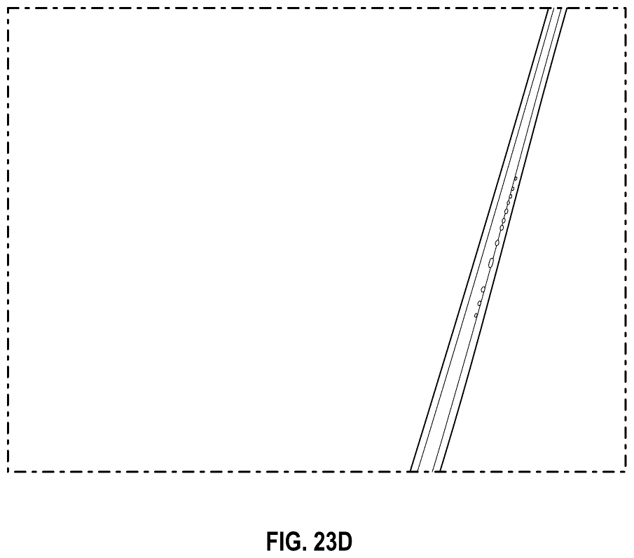

FIG. 22 is a diagram of a test system that was used for detecting the removal of air bubbles between concentrically stacked catheters. The test system included an inner catheter 2108 positioned within an interior lumen of an outer catheter 2106 in a concentric stack. The outer catheter 2106 was coupled to a rotating hemostasis valve 2104. The hemostasis valve 2104 was coupled to a syringe 2102 so that fluid injected using the syringe would flow through the lumen between the inner catheter 2108 and the outer catheter 2016. In the test system, the inner catheter 2108 had a diameter of about 0.071 inches. The outer catheter 2106 had a diameter of about 0.088 inches. The outer catheter 2106 was transparent to permit visualization of bubbles within the lumen. A distal end of the outer catheter 2108 allowed for small volumes of fluid to exit the outer catheter. FIG. 23A is a photograph showing the catheter 2106 and catheter 2108 in a concentric stack, prior to injection of fluid. FIG. 23D is an illustration thereof.

### Example 1

In a first example, the syringe 2102 was used to inject water at a constant pressure of about 150 psi through the hemostasis valve 2104 without moving the catheter 2106 or the catheter 2108. FIG. 23B is a photograph showing the catheter 2106 and catheter 2108 following the injection of water. FIG. 23E is an illustration thereof. As shown in FIG. 23B, bubbles are present within the lumen between the catheter 2106 and the catheter 2108.

### Example 2

In a second example, the syringe 2102 was used to inject water at a constant pressure of about 150 psi through the hemostasis valve 2104. Shortly after beginning to inject water, axial reciprocal movement of the inner catheter 2108 was performed for about 10 seconds. The reciprocal movement was performed at a frequency of about 1 Hz (or less) and a stroke length of about 20 mm (or more). FIG. 23C is a photograph showing the catheter 2106 and the catheter 2108 following the axial reciprocal movement. FIG. 23F is an illustration thereof. As shown in FIG. 23C, the lumen between the catheter 2106 and the catheter 2108 was substantially free of bubbles.

### Example 3

In a third example, an outer catheter having a diameter of about 0.071 inches and an inner catheter having a diameter of about 0.035 inches were used in the test system 2100 instead of the outer catheter 2106 and the inner catheter 2108 described with respect to Examples 1 and 2. A syringe 2102 was used to inject water at a constant pressure of about 150 psi through a hemostasis valve 2104 coupled to the outer catheter. Shortly after beginning to inject water, axial reciprocal movement of the inner catheter was performed for about 10 seconds. The reciprocal movement was performed at a frequency of about 1 Hz (or less) and a stroke length of about 20 mm (or more). Following the axial reciprocal movement, the lumen between the outer and inner catheters was found to be substantially free of bubbles by visual inspection.

### Control System

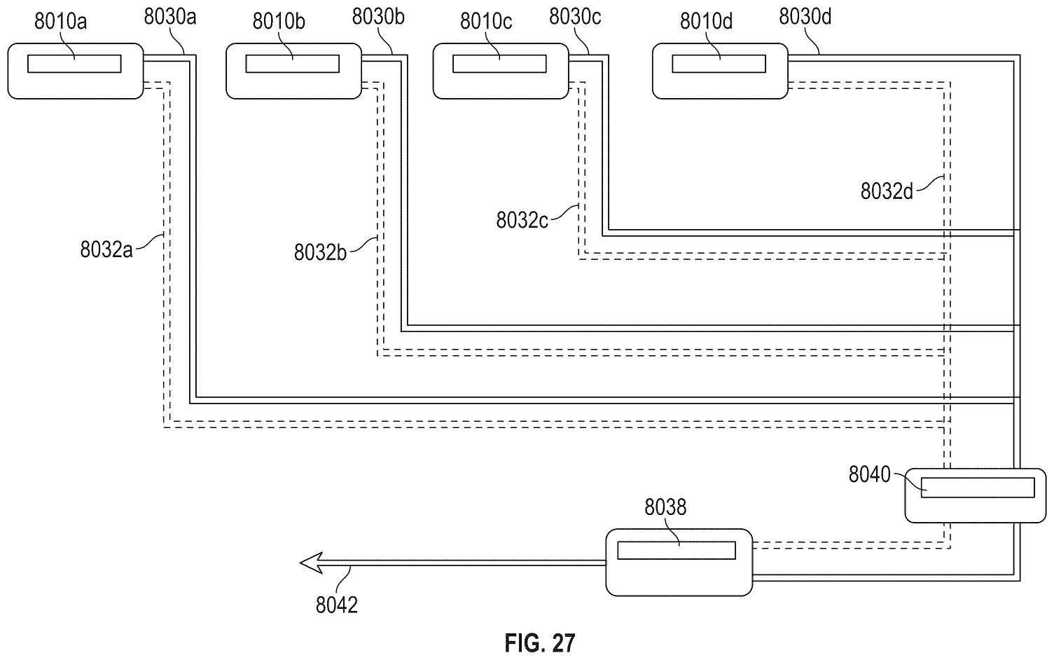

FIG. 28 illustrates a schematic view of an example of a control system 4000 that may be used to electronically control the systems and components described herein and/or perform the methods described herein. The control system 4000 may be configured to automatically adjust various motors, hub adapters, hubs, interventional devices, fluidics components (e.g., valves, pumps, etc.), and/or any other components described herein in response to commands input by an operator such as a physician. In response to command inputs by an operator, the control system 4000 may cause a series of responsive events to automatically occur.

In certain embodiments, the control system 4000 can include one or more processors 4002. The one or more processors 4002 can be configured to automatically adjust the various system components described herein in response to commands input by an operator, for example, using one or more controls 4004 of the control system 4000. A single control 4004 is shown in FIG. 28. However, any suitable number of controls may be provided to correspond to various functions of the systems described herein. For example, in certain embodiments, each interventional device may have its own unique control 4004 or set of controls 4004 that can control various functions of the interventional device (e.g., axial movement, rotational movement, supply of fluids (e.g., saline, contrast, etc.), aspiration, etc.).

In certain embodiments, one or more controls 4004 may control priming functions for one or more interventional devices. For example, one or more controls 4004 can be operated to cause the interventional devices to perform a priming procedure, as described for example, with reference to FIGS. 20A-C. For example, one or more controls 4004 can be operated to cause axial movement of one or more interventional devices relative to one or more other interventional devices (e.g., by causing axial movement of corresponding hubs and/or hub adapters). One or more controls 4004 can be operated to cause introduction of fluid into the lumen of an interventional device to prime the interventional device.

In certain embodiments, one or more controls 4004 may be operated to cause the interventional devices to perform a priming procedure, as described for example, with reference to FIGS. 21A-21B. For example, one or more controls 4004 can be operated to cause reciprocal movement (e.g., axial and/or rotational reciprocal movement) of one or more interventional devices relative to one or more other interventional devices (e.g., by causing reciprocal movement of corresponding hubs and/or hub adapters). One or more controls 4004 can be operated to cause introduction of fluid into the lumen of an interventional device to prime the interventional device (e.g., during relative reciprocal movement).

The processor 4002 may receive signals from the one or more controls 4004 and in response, initiate corresponding actions in the components of the systems described herein. For example, the processor 4002 may be configured to generate output signals that cause responsive actions to be performed by the components of the described herein.

While the foregoing describes robotically driven interventional devices and manually driven interventional devices, the devices may be manually driven, robotically driven, or any combination of manually and robotically driven interventional devices, as will be appreciated by those of skill in the art in view of the disclosure herein.

The foregoing represents one specific implementation of a robotic control system. A wide variety of different robotic control system constructions can be made, for supporting and axially advancing and retracting two or three or four or more assemblies to robotically drive interventional devices, as will be appreciated by those of skill in the art in view of the disclosure herein.

While the foregoing describes interventional devices that are driven by a drive table, other suitable robotic drive systems or mechanisms may be used to drive the interventional devices, as will be appreciated by those of skill in the art in view of the disclosure herein.

Various systems and methods are described herein primarily in the context of a neurovascular access or procedure (e.g., neurothrombectomy). However, the catheters, systems (e.g., drive systems), and methods disclosed herein can be readily adapted for any of a wide variety of other diagnostic and therapeutic applications throughout the body, including particularly intravascular procedures such as in the peripheral vasculature (e.g., deep venous thrombosis), central vasculature (pulmonary embolism), and coronary vasculature, as well as procedures in other hollow organs or tubular structures in the body.

In any embodiments disclosed herein, a robotic drive system can further include a magnet (also referred to herein as a driven magnet) on each of one or more of the hubs, including without limitation, on a guidewire hub, an access catheter hub, and a guide catheter hub. Each of the driven magnets can be configured to cooperate with a corresponding drive magnet positioned on each of one or more of the hub adapters. In some embodiments, the system can be configured such that the driven magnet moves in response to a movement of the corresponding drive magnet. In some embodiments, the system can be configured to have a magnet on one of the hub and the hub adapter and a ferrous object on the other of the hub and the hub adapter. In some embodiments, the drive magnets can each be independently axially movably carried by a support table. In some embodiments, without limitation, the drive magnets can be located outside of the sterile field, separated from the driven magnets by a barrier, and the driven magnets may be located within the sterile field. The barrier may comprise a tray made from a thin polymer membrane, or any membrane of non-ferromagnetic material, or otherwise.

In any embodiments disclosed herein, one or more of or each of the magnets can be permanent magnets, rare earth magnets, or neodymium magnets. In any embodiments disclosed herein, one or more of or each of the magnets can be rare earth magnets, neodymium magnets, anisotropic ferrite magnets, or otherwise. In any embodiments disclosed herein, one or more of or each of the magnets can include rare earth magnetic material, neodymium magnetic material, anisotropic ferrites, or otherwise.

In some embodiments, one or more of the hubs and/or hub adapters disclosed herein can have one or more magnets that can be used with a magnetic field sensor to provide information to the user of the system regarding a position of the hub relative to a corresponding hub adapter, regarding forces applied to a hub, and other information. More details about this are provided below.

### Force and Coupling Sensors:

In some embodiments, any of the hubs or pucks disclosed herein (e.g., guidewire hub 26, access catheter hub 28, guide catheter hub 30, hub 36, hub 250, guidewire hub 2909, insert or access catheter hub 2910, procedure catheter hub 2912, and guide catheter hub 2914) and/or hub adapters disclosed herein (e.g., hub adapter 48), can include any of a variety of sensors to measure parameters associated with the hubs, instruments coupled with the hubs, etc. Such parameters can include, for example and without limitation, forces applied to the hubs and/or displacement of a hub relative to its corresponding hub adapter. In some embodiments, the parameters can include magnetic field magnitude and/or magnetic field direction.

The parameters may be used by the robotic control system to determine a state or condition of one or more of the hubs, the hub adapters, the hub/hub adapter pairs, the interventional devices, and/or the robotic control system or the occurrence of an event during a procedure. For example, as described herein, the parameters may be used to determine displacement or disconnection between a hub and a hub adapter, excessive forces on an interventional device and/or hub, excessive forces (e.g., frictional forces) between interventional devices, excessive energy storage in interventional devices, etc. In some embodiments, the parameters for multiple hubs, hub adapters, hub/hub adapter pairs, and/or interventional devices can be compared to determine a state or condition of one or more of the hubs, the hub adapters, the hub/hub adapter pairs, the interventional devices, and/or the robotic control system or the occurrence of an event during a procedure.

In some embodiments, one or more sensors may measure magnetic field vectors. The sensors can measure the magnitude of the magnetic field in one or more directions (e.g., x, y, and z directions). In some embodiments, the magnetic field measurements may be used to determine a magnitude and/or direction of displacement of the hub from the corresponding hub adapter (e.g., based on measured magnetic field vectors or changes in measured magnetic field vectors).

In some embodiments, the magnetic field sensor or sensors can be used to measure the magnetic field strength of a magnetic coupling between a hub and a respective hub adapter. In some embodiments, the magnetic field sensor or sensors can be positioned on the hub or the hub adapter and can be used to measure the magnetic field strength of a magnet (also referred to herein as a sensor target magnet) on the other of the hub and the hub adapter (i.e., whichever component does not have the magnetic field sensor). For example and without limitation, the magnetic field sensor can be positioned on any one of or all of the hub adapters and the magnet that produces the magnetic field that is sensed by the magnetic field sensor can be positioned on any one of or all of the corresponding hubs at a position so that its magnetic field will be sensed by the magnetic field sensor (e.g., a position aligned with the position of the magnetic field sensor) when the hub is in a desired position of alignment with the hub adapter. In any embodiments of the robotic control system disclosed herein, the magnet that produces the magnetic field that is sensed by the magnetic field sensor can be a different magnet than the drive magnet and different than the driven magnet. In some embodiments, the magnet that produces the magnetic field that is sensed by the magnetic field sensor can also be used for other purposes, such as for example and without limitation, transmission of axial forces from one or more of the hub adapters to the corresponding hubs.

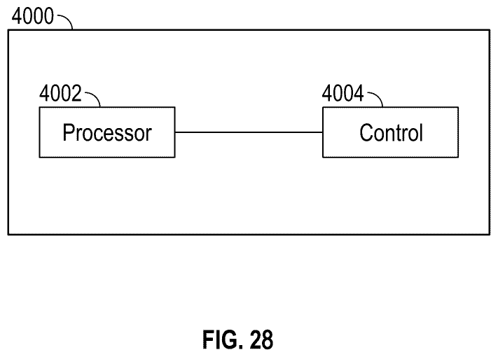

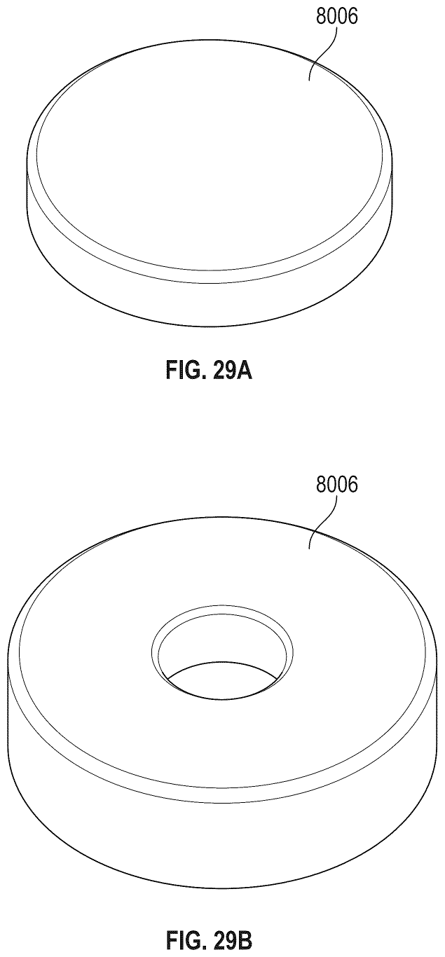

In any embodiments disclosed herein, the magnet that produces the magnetic field that is sensed by the magnetic field sensor can be a disc shaped magnet (for example and without limitation, like magnet 8006 shown in FIG. 29A), a cylindrically shaped magnet, a ring shaped magnet (for example and without limitation, like magnet 8006 shown in FIG. 29B), or any other suitable type of magnet. The ring-shaped magnets are also referred to herein as ring magnets. In some embodiments, one or more of the magnets that produce the magnetic field that is sensed by the magnetic field sensor (for example, the ring magnets) can have an opening or hole through a center thereof in an axial direction and have a circular shape at a perimeter of the magnet. In any embodiments disclosed herein, the driving magnet of at least one hub adapter and the driven magnet of the corresponding hub can be ring magnets. In any embodiments disclosed herein, the driving magnet of every hub adapter of the robotic control system and the driven magnet of the corresponding hubs can be ring magnets.

In certain embodiments having a ring magnet, as the ring magnet passes the magnetic field sensor (e.g., passes over the magnetic field sensor), the ring magnet can provide additional information about the position of the magnet relative to the sensor. In certain embodiments having a ring magnet, the ring magnet can be radially or concentrically magnetized. For example and without limitation, a north pole of the magnet can be at an innermost portion of a ring magnet, which can be surrounded by a south pole, such that an outermost perimeter of the ring magnet can magnetically be a south pole. Conversely, in some embodiments of the ring magnet, a south pole of the magnet can be at an innermost portion of a ring magnet, which can be surrounded by a north pole, such that an outermost perimeter of the ring magnet can magnetically be a north pole. Knowing the polarity of the ring magnet, one can therefore gather additional information not provided by a solid cross-section magnet—e.g., the system can be configured to gather polarity information to get additional information related to the position of the magnet relative to the sensor. For example, in such embodiments, as the ring magnet passes the magnetic field sensor (e.g., passes over the magnetic field sensor), the magnetic field sensor can detect the orientation of the magnetic field. Knowing the polarity of the ring magnet, this information may be used to determine the position of the magnet relative to the sensor. In some embodiments, the orientation information may be used along with magnetic field strength information to determine the position of the magnet relative to the sensor. In other embodiments, ring magnets may have alternative magnetization directions that may provide information to the magnetic field sensor regarding the orientation of the magnetic field that may be used to determine the position of the magnet relative to the sensor. For example, in certain embodiments, the ring magnets may be diametrically magnetized. In certain embodiments, other magnets, such as disc magnets, may have magnetization directions that provide information to the magnetic field sensor regarding the orientation of the magnetic field that may be used to determine the position of the magnet relative to the sensor.

In any embodiments disclosed herein, the hub and a respective hub adapter can each have a magnet, each magnet being configured to attract the magnet of the other of the hub and the hub adapter. Alternatively, in any embodiments disclosed herein, the hub or a respective hub adapter can have a magnet and the other of the hub and the hub adapter can have a ferromagnetic object configured to magnetically couple with the magnet.

In any embodiments, the sensor or sensors can be used to measure the magnetic field strength between the magnets or between the magnets and ferromagnetic objects of the hub and hub adapter pair. In some embodiments, a magnetic field magnitude (or magnetic field vector magnitudes) of the magnetic field between the respective magnets or the magnet and the ferromagnetic object of a hub and hub adapter pair in a baseline position can be measured to determine and establish a baseline magnitude (or baseline vector magnitudes) of the magnetic force between the hub and hub adapter of each hub and hub adapter pair. For example and without limitation, the baseline magnitude (or baseline vector magnitudes) can be a magnitude (or a set of baseline vector magnitudes) of the magnetic field of the hub and hub adapter pair when the hub and hub adapter are in a baseline position—e.g., when the hub and hub adapter are aligned in the axial direction. In some embodiments, this can be the position of the hub and hub adapter when the hub and hub adapter are magnetically coupled to one another and no external forces (or, in some embodiments, when no other significant external forces) are acting on the hub or hub adapter other than the magnetic coupling force. The baseline magnitude (or set of baseline vector magnitudes) may be the highest value of the magnetic field strength for each pair.

In some embodiments, the system can be configured to determine or approximate a displacement of a hub from the baseline position of the hub (referred to herein as ΔP or as offset distance), based on a difference between the actual (e.g., measured) magnetic field magnitude (or vector magnitudes) and the baseline magnitude (or set of baseline vector magnitudes) of the magnetic coupling force (i.e., baseline magnitude minus actual magnetic field magnitude, referred to herein as ΔF). In some embodiments, empirical data related to the difference between the actual magnetic coupling force magnitude (or vector magnitudes) and the baseline magnitude (or set of baseline vector magnitudes) of the magnetic coupling force at a range of displacement positions can be gathered. These values can be stored in a lookup table, in some embodiments. This information can be used to determine a position or a displacement of the hub adapter relative to the respective hub (i.e., the ΔP value) based on the ΔF value.

In some embodiments, the system can be configured to have a lookup table of data of displacement values between a respective hub and hub adapter relative to a difference between a respective magnetic coupling force magnitude (or vector magnitudes). The lookup table can also have data related to the baseline magnitude (or set of baseline vector magnitudes) of the magnetic coupling force. In some embodiments, the lookup table can be based on empirical data for a hub and hub adapter pair derived from on actual measurements of displacement between a respective hub and hub adapter relative to a difference between a respective magnetic coupling force magnitude (or vector magnitudes) and the baseline magnitude (or set of baseline vector magnitudes) of the magnetic coupling force.

In some embodiments, mapping of magnetic field vector data to displacement is not disturbed by friction between the hub and the sterile barrier.

In some embodiments, the system can be configured to determine or approximate an external force that is applied to a hub. For example and without limitation, the external force can be determined based on a difference between the measured magnetic force magnitude (or vector magnitudes) between the hub and hub adapter and the baseline magnitude (or set of baseline vector magnitudes). From this, some embodiments can be configured to calculate the external force acting on the hub and the displacement ΔP between the hub and hub adapter. Some embodiments, can be configured to calculate the direction of external forces acting on the hub (e.g., based on a difference measured and baseline magnetic field vectors). Therefore, as mentioned, in some embodiments, the magnetic field measurements may be used to determine a magnitude and/or direction of external forces acting on the hub (e.g., based on measured magnetic field vectors or changes in measured magnetic field vectors).

Additionally or alternatively, in some embodiments, a hub adapter baseline magnetic field value (e.g., magnitude) or magnetic field vector values (e.g., magnetic field vector magnitudes) can be determined when no hub is coupled with the hub adapter. The system can be configured to determine that a hub is coupled to the hub adapter based on a difference between the actual (e.g., measured) magnetic field magnitude (or vector magnitudes) and the hub adapter baseline magnetic field magnitude (or set of baseline vector magnitudes). In some embodiments, the system can be configured to determine or approximate a displacement of a hub from the baseline position of the hub (referred to herein as ΔP or as offset distance) or an external force that is applied to a hub based at least in part on the hub adapter baseline magnetic field magnitude (or set of baseline vector magnitude), for example, based on a difference between the actual (e.g., measured) magnetic field magnitude (or vector magnitudes) and the hub adapter baseline magnetic field magnitude (or set of baseline vector magnitudes). In other embodiments, a hub baseline magnetic magnitude (or set of baseline vector magnitudes) can be determined when the hub is not coupled to the hub adapter and may be used in the same or similar manner as the hub adapter baseline magnetic field magnitude (or set of hub adapter baseline vector magnitudes). Data from the sensors may be used to determine other characteristics of the system, such as for example, whether a hub has contacted and is pushing against another hub and/or whether a hub is magnetically tethered to a hub adapter. For example, whether a hub has contacted and is pushing against another hub and/or whether a hub is magnetically tethered to a hub adapter may be determined based on a determined displacement between the hub and the hub adapter and/or a determined force acting on the hub.

In some embodiments, force measurements and/or magnetic field magnitude and/or direction measurements from one or more of the sensors can be used to determine that a movement of one or more of the hubs has been impeded or prevented. This can be the result of tension in fluid lines or electrical lines coupled with the hubs (e.g., the fluid lines are snagged or are being stretched), one or more of the hubs physically contacting another hub or object (e.g., a hub is pushing another hub), faulty condition of the hub, forces acting on an intervention device coupled to the hub, and/or other physical impediments to the movement of the hub.

In some embodiments, force measurements and/or magnetic field magnitude and/or direction measurements from one or more of the sensors can be used to determine that a range of motion of the anti-buckling components (e.g., any of the anti-bucking components disclosed herein) is approaching an out of range position or that the range of motion is being exceeded.

In some embodiments, force measurements and/or magnetic field magnitude and/or direction measurements from one or more of the sensors can be used to determine that one or more of the hubs has become detached from the respective hub adapter.

In some embodiments, force measurements and/or magnetic field magnitude and/or direction measurements from one or more of the sensors can be used to determine that friction forces internal to the body are large or excessive. In any embodiments disclosed herein, the robotic control system can be configured to provide a warning to the user when an abnormal condition (such as any of the abnormal or potentially abnormal conditions discussed herein) is present. In some embodiments, the warning can include visual alerts on a graphical user interface or other user interface, audible alerts to the user, haptic feedback, and any combination of the foregoing.

In some embodiments, active real-time or near real-time force sensing can be used for each interventional device (or a subset of interventional devices) to detect energy storage in compression, tension, and/or rotational shear which can be generated by friction between the interventional devices. Energy storage may occur in an interventional device, for example, axially, due to cumulative friction along the shaft of the interventional device. Clinically, this can present various problems. For example, desired, commanded motions may not transmit to the tip of the shaft due to cumulative friction along the shaft. Also, stored energy may release when frictional forces are overcome, which can result in significant uncommanded (i.e., inadvertent) motion of the shafts. In some embodiments, the energy storage may be relieved by a user performing corrective actions (e.g., using a push/pull technique on one or more of the interventional devices) to reduce the risk of inadvertent uncommanded motion due to energy release.

In some embodiments, force patterns derived from the magnetic field magnitude information gathered by the system can also be used to execute control functions. For example, in some embodiments, force patterns derived from the magnetic field magnitude information can be used to execute control functions to compensate for or relieve energy storage (e.g., axially in a shaft due to cumulative friction along the shaft of an interventional devices). For example, in some embodiments, force sensing data can be used by any embodiments of the robotic control system described herein to detect energy storage and provide a warning to user (for example, if the amount of energy stored exceeds a threshold value). In some embodiments, the drive system may automatically adjust (for example, using control algorithms) the interventional devices. For example and without limitation, some embodiments of the drive system may automatically adjust the interventional devices via axial movement of interventional devices in which an amount of stored energy exceeds a threshold value to relieve the energy storage.

In some embodiments, the robotic control system can be configured to execute control functions to mitigate one or more abnormal conditions. For example, some embodiments of the robotic control system can be configured to override the user control of one or more hub adapters to prevent further movement of the one or more hub adapters. In some embodiments, the robotic control system can be configured to override the user control of one or more hub adapters and move a hub adapter and, consequently, a coupled or tethered hub (e.g., a first hub) away from another hub that the first hub is interfering with, or to move the first hub in a direction that reduces a force on the first hub or on a hub that the first hub is interfering with.

In some embodiments, the robotic control system can have one or more sensors in the hub adapter and/or corresponding hub configured to detect whether the hub is coupled with the hub adapter. This can include determining whether the hub is magnetically coupled to and/or aligned with the hub adapter.

In some embodiments, the robotic control system can have a sensor such as, but not limited to, a magnetometer positioned in or coupled to the hub adapter, to detect an existence and/or magnitude and/or direction of a magnetic field created by a magnet positioned in or coupled to the hub. In some embodiments, the robotic control system can have a sensor such as, but not limited to, a magnetometer positioned in or coupled to the hub, to detect an existence and/or magnitude and/or direction of a magnetic field created by a magnet positioned in or coupled to the hub adapter. Detection of the existence and/or magnitude and/or direction of the magnetic field can be used to determine whether the hub is magnetically tethered to the hub adapter.

In some embodiments, the magnitude and/or direction of a magnetic field created by a magnet positioned in the hub or hub adapter can be measured by the sensor to determine a displacement (i.e., an offset distance), if any, between the hub and the hub adapter. In some embodiments, the magnitude and/or direction of a magnetic field created by a magnet positioned in the hub or hub adapter can be measured by the sensor to determine a magnitude of force(s) being applied to the hub.

In some embodiments, the sensors can include an RFID reader positioned on or coupled with one of the hub and the hub adapter and configured to interrogate an RFID tag positioned on or coupled with the other of the hub and the hub adapter, to verify that the hub and hub adapter are coupled (e.g., magnetically coupled). For example, in some embodiments, the RFID reader is positioned on or coupled with the hub and the RFID tag is positioned on or coupled with the hub adapter. In some embodiments, the RFID reader is positioned on or coupled with the hub adapter and the RFID tag is positioned on or coupled with the hub. The RFID reader can be used in combination with a magnetic sensor, such as a magnetometer, to verify that the magnetic field sensed by the magnetic sensor is from a corresponding hub or hub adapter instead of a different magnetic object.

Features and Benefits of Force and/or Coupling Sensors:

Some embodiments of the robotic control system disclosed herein having one or more force and/or coupling sensors can have at least the following features or otherwise be configured to perform the following functions.

In some embodiments, the robotic control system can be configured to measure (e.g., continuously or near continuously) magnetic field strength and/or direction between the hub and the hub adapter and provide hub decoupling sensing and/or warnings to the user. For example, if it is determined that a hub will become decoupled from a hub adapter at a particular relative displacement value (also referred to herein as a displacement threshold value), the robotic control system can be configured to communicate an alert to a user if the relative displacement between the hub and the hub adapter reaches a particular predetermined percentage of the displacement threshold value, such as 70% of the displacement threshold value, approximately 70% of the displacement threshold value, 80% of the displacement threshold value, approximately 80% of the displacement threshold value, from 60% of the displacement threshold value or approximately 60% or less than 60% of the displacement threshold value to 90% of the displacement threshold value or approximately 90% of the displacement threshold value or more than 90% of the displacement threshold value, or any value within any of the foregoing ranges or other suitable predetermined percentage.

Alternatively, if it is determined that a hub will become decoupled from a hub adapter at a particular relative force value (also referred to herein as a force threshold value or breakaway force), the robotic control system can be configured to communicate an alert to a user if the net external forces acting on the hub reaches a predetermined percentage of the breakaway force, such as 70% of the breakaway force, approximately 70% of the breakaway force, 80% of the breakaway force, approximately 80% of the breakaway force, from 60% of the breakaway force or approximately 60% or less than 60% of the breakaway force to 90% of the breakaway force or approximately 90% of the breakaway force or more than 90% of the breakaway force, or any value within any of the foregoing ranges or other suitable predetermined percentage.

In some embodiments, the robotic control system can be configured to increase an intensity of the alert to the user as the relative displacement value or the net external force on the hub increases, such as increasing a size of a warning symbol (which can be a triangle, an arrow, or any other desired shape), increasing a color tone or opacity of a warning symbol, changing the color of the warning symbol, increasing a volume level or changing a pitch of an audible warning, and/or providing other levels of warning.

In some embodiments, the robotic control system can be configured to warn a user regarding a risk of decoupling between the hub and the hub adapter at a lower predetermined percentage of the displacement threshold value (e.g., 50% or approximately 50% or less than 50% of displacement threshold value before decoupling) or a lower value of the net external force value acting on the hub (e.g., 50% of the breakaway force, approximately 50% of the breakaway force) if a hub adapter and/or hub is moving at a higher speed. This can allow more time to the user to react to mitigate the relative displacement or other issue.

Additionally, in some embodiments, the robotic control system can be configured to impede a movement of the hub adapter if the relative displacement reaches a threshold value (e.g., 80% or approximately 80% or at least 80% of the displacement threshold value, or 70% or approximately 70% or at least 70% of the displacement threshold value, or from 60% of the displacement threshold value or approximately 60% or less than 60% of the displacement threshold value to 90% of the displacement threshold value or approximately 90% of the displacement threshold value or more than 90% of the displacement threshold value, or any percentage within any of the foregoing ranges) by increasing a resistance force on the hub adapter and/or by slowing a movement of the hub adapter. In some embodiments, the robotic control system can increase such resistance force or further slow the movement of the hub adapter if the relative displacement reaches a second threshold value (e.g., 90% or approximately 90% of total permissible relative displacement before decoupling), and so on.

Similarly, in some embodiments, the robotic control system can be configured to impede a movement of the hub adapter if the net external force acting on a hub reaches a threshold value (e.g., 80% or approximately 80% of the breakaway force, or 70% or approximately 70% of the breakaway force, or from 60% of the breakaway force or approximately 60% or less than 60% of the breakaway force to 90% of the breakaway force or approximately 90% of the breakaway force or more than 90% of the breakaway force, or any percentage within any of the foregoing ranges) by increasing a resistance force on the hub adapter and/or by slowing a movement of the hub adapter. In some embodiments, the robotic control system can increase such resistance force or further slow the movement of the hub adapter if the net external force acting on a hub reaches a second threshold value (e.g., 90% or approximately 90% of breakaway force), and so on. In some embodiments, the robotic control system can be configured to impede a movement of the hub adapter in a direction that would increase the net external force acting on a hub if the net external force acting on the hub reaches the threshold value

As mentioned, some embodiments of the robotic control system disclosed herein can be configured to measure magnetic field strength and/or direction (e.g., continuously or near continuously) between the hub and the hub adapter and provide imminent hub detachment warnings. In some embodiments, the robotic control system can be configured to measure magnetic field strength and/or direction (e.g., continuously or near continuously) between the hub and the hub adapter and provide excessive force (e.g., excessive insertion force) warnings and/or take other corrective action. For example, corrective actions may include preventing further movement of the hub adapter in a direction that would increase the external force on the hub or interventional device, moving the hub adapter in a direction that would reduce the external force on the hub or interventional device, detaching the hub from the hub adapter, or any other suitable corrective action. In some embodiments, the robotic control system can recommend corrective maneuvers to a user to reduce loads on one or multiple hubs.

In some embodiments, the robotic control system can be configured to communicate an alert to a user if an axial force applied to the hub is greater than 2 N, greater than approximately 2 N, greater than 3 N, greater than approximately 3 N, greater than 5 N, greater than approximately 5 N, greater than 10 N, greater than approximately 10 N, greater than 15 N, greater than approximately 15 N, or any other suitable value.

Some embodiments of the robotic control system disclosed herein can be configured to measure magnetic field strength and/or direction (e.g., continuously or near continuously) between the hub and the hub adapter and provide real-time or nearly instantaneous force data to the user. In some embodiments, the robotic control system can be configured to measure magnetic field strength and/or direction (e.g., continuously or near continuously) between the hub and the hub adapter and provide data (e.g., real-time or nearly instantaneous data) to a user regarding forces exerted on an interventional device (e.g., catheter or guidewire) coupled to the hub. In some embodiments, the robotic control system can be configured to measure magnetic field strength (e.g., continuously or near continuously) between the hub and the hub adapter and provide data (e.g., real-time or nearly instantaneous data) to a user regarding a magnitude of axial torque built up in an interventional device (e.g., a catheter, such as an insertor or access catheter, or guidewire) coupled to the hub. In some embodiments, torque can be measured by measuring changes in the magnetic field (e.g., the magnitude of the magnetic field) in a direction that is perpendicular to the axis of the shaft.

Some embodiments of the robotic control system disclosed herein having one or more force and/or coupling sensors can have at least the following benefits. In some embodiments, the robotic control system can determine if a hub is close to detaching (i.e., magnetically decoupling) from the respective hub adapter so that appropriate warnings can be communicated to the user and/or or corrective action can be taken. In some embodiments, the robotic control system can determine if a hub has detached (i.e., magnetically decoupled) from the respective hub adapter so that appropriate warnings can be communicated to the user and/or or corrective action can be taken. In some embodiments, the robotic control system can determine the existence and/or magnitude of external forces on the hub so that appropriate warnings can be communicated to the user and/or or corrective action can be taken. In some embodiments, the robotic control system can be configured to determine if the insertion forces acting through the magnetic coupling exceed a predetermined threshold value so that appropriate warnings can be communicated to the user and/or or corrective action can be taken.

Some embodiments of the robotic control system disclosed herein can be configured to detect pre-determined patterns related to undesired clinical scenarios (for example, for one or multiple hubs/interventional devices) and provide warnings and/or take other corrective action. For example, if a pattern is recognized indicating that adjacent interventional device shafts are undergoing simultaneous forces in opposite directions, the system may provide a warning or automatically take corrective action, for example, by unloading either or both interventional devices to reduce the energy storage generated by frictional interplay between them.

As described, in certain embodiments, a robotic control system can be configured to perform magnetic force sensing for multiple hub/hub adapter combinations. In certain embodiments, the robotic control system can analyze (e.g., compare) data for multiple hub/hub adapter pairs (e.g., magnetic field magnitude, magnetic field vector magnitude, force, displacement, direction of force, direction of displacement, and/or changes in any of the foregoing over time) to determine a status or condition of one or more hub/hub adapter pairs, one or more interventional devices, or of the robotic control system, or the occurrence of one or more events within a procedure. For example, if the system determines that the forces acting on a first hub, such as hub 2914, are the same or similar in magnitude and opposite in direction from the forces acting on a second hub, such as hub 2912, the system may determine that a large amount of friction is present between the interventional device coupled to the first hub, such as guide catheter 2906, and the interventional device coupled to the second hub, such as procedure catheter 2904. As another example, if the system determines that the forces on each hub of an interventional device assembly are increasing as the interventional device assembly is inserted into more distal and tortuous vasculature, it may be determined that friction in the vasculature is a source of additional force on the hubs.

Additionally, in some embodiments, having force and/or displacement data related to one or more of the hub and hub adapter pairs can provide useful information to assist during setup of the equipment before a procedure. For example and without limitation, in some embodiments, one or more sensors can be configured to determine whether a hub is oriented correctly relative to a hub adapter and provide warnings or other output configured to alert a user that a position and/or orientation of a hub relative to a hub adapter may be incorrect or outside a threshold tolerance range (e.g., such as 20% of the displacement threshold value, approximately 20% of the displacement threshold value, 10% of the displacement threshold value, approximately 10% of the displacement threshold value, from 10% of the displacement threshold value or approximately 10% or less than 10% of the displacement threshold value to 30% of the displacement threshold value or approximately 30% of the displacement threshold value or more than 30% of the displacement threshold value, or any value within any of the foregoing ranges or other suitable predetermined percentage.

Additionally, in some embodiments, one or more sensors can be used to determine whether a hub is oriented correctly relative to a hub adapter and provide warnings or other output configured to impede or block use of the system before the error condition is corrected. In some embodiments, the robotic control system can be configured to identify to the user or operator which hub may be outside of range or tolerance in terms of relative displacement with respect to the corresponding hub adapter, orientation relative to a hub adapter, which hub may be outside of range or tolerance for pre-procedure setup for net forces acting on a hub, or other parameters. In some embodiments, the robotic control system can be configured to indicate to a user or provide feedback to a user to help the user to understand a magnitude or degree of misalignment, a direction of misalignment, a magnitude or degree of net external forces acting on a hub, an orientation of the net external forces acting on a hub, or other parameters. Additionally, some embodiments of the robotic control system can be configured to assess and provide feedback to a user that relative displacements of hubs relative to hub adapters, net external forces, and/or orientation are all within acceptable ranges or values, that initial setup has been completed correctly, and/or other working parameters of the robotic control system are within acceptable (e.g., within predetermined or threshold) ranges.

### Measurement of Offset Between Hub and Hub Adapter and Forces Applied to Hub:

As described herein, some embodiments of the robotic control system can have sensors in the hub adapter and/or hub configured to measure a force that is being applied to the hub. In some embodiments, and as described herein with respect to some embodiments, magnetic field vector data can be used to calculate the force acting on hub from the magnetic coupling between the hub and the hub adapter and/or a separate external force acting on the hub. The calculated force can in some embodiments be proportional to, if not equal to, the net resulting force of all external forces acting on the hub.

Calculating forces being applied to the hub can help prevent the hub being sheared off of or untethered from the hub adapter, which can occur if a movement of the hub is restrained or impeded, but the hub adapter continues to move relative to the hub.

In some embodiments, the user may feel a resistance to movement of the hub adapter relative to the hub due to the magnetic force between the hub and the hub adapter, when coupled (e.g., via haptic feedback).

In some embodiments, the sensor, which can be a magnetometer, can continuously (or near continuously) measure a magnitude and/or direction of the magnetic field (e.g., by measuring a magnetic field vector). In some embodiments, the sensor can provide continuous (or near continuous) feedback to the user of an amount of offset between the hub adapter and the hub and/or a magnitude and/or direction of any external forces applied to the hub.

Any embodiments of the robotic control system disclosed herein can include a magnetometer device in the hub adapter or hub and a corresponding magnet (e.g., a rare earth magnet) in the other of the hub adapter and the hub. The relative displacement of hub and hub adapter can be characterized based on changes in the magnetic field strength detected by the magnetometer. In certain embodiments, data regarding the magnetic field strength and/or direction generated by the magnet opposite to a magnetometer can be used in some embodiments to measure either the vertical or horizontal displacement of the hub relative to the hub adapter.

FIG. 24 is a schematic representation of a portion of robotic control system 8000 showing a hub (which can be any of the embodiments of the hub disclosed herein, including without limitation hub 2910) and a hub adaptor (which can be any of the embodiments of the hub adapter disclosed herein, including without limitation hub adapter 8004b). The hub 2910 can include one or more magnets 8006, and the hub adapter 8004 can include one or magnets 8007 to magnetically couple the hub 2910 and the hub adapter 8004.

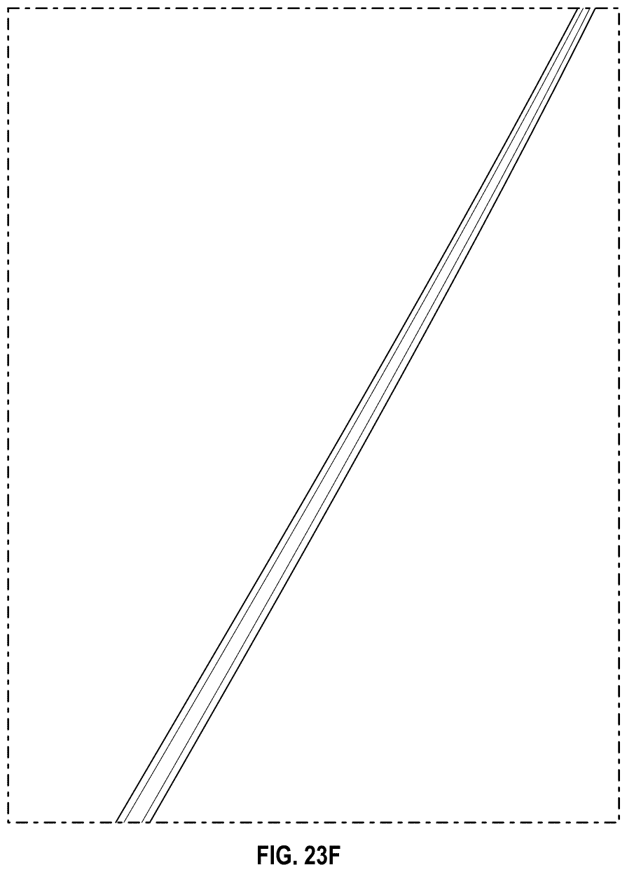

In some embodiments, the magnetic coupling between the hub 2910 and the hub adapter 8004 is elastic. Magnetic field strength data can be used to calculate elastic forces across the magnetic coupling between the hub 2910 and the hub adapter 8004. Due to the elastic nature of the coupling, the magnetic coupling may be modeled as a spring coupling, as shown in FIG. 25, which shows a schematic representation of a hypothetical system having a spring positioned between the hub 2910 and the hub adapter 8004.

A relative displacement 8020 between the hub 2910 and the hub adapter 8004 in a z direction (i.e., a proximal-distal direction or axial direction of movement of the hub) is represented in FIG. 24 and FIG. 25 by Δz. As shown in FIG. 24, the relative displacement 8020 between the hub 2910 and the hub adapter 8004 can be caused by force F_{hub} acting on the hub 2910 and/or force F_{adapter} acting on the hub adapter 8004.

In some embodiments, the magnetometer can be configured to measure magnetic field strength in a direction of axial movement of the of the hub 2910 (i.e., parallel to Δz). In some embodiments, the force acting on the hub 2910, F_{hub}, can be characterized by the following equation:

$${F}_{h u b} = {k}_{m a g} ( Δ z ( {B}_{z} ) ) \cdot {B}_{z}$$

in which Δz is the relative displacement of a fixed point on the hub 2910 relative to a fixed point on the hub adapter 8004, l_{mag} is an effective spring constant between the hub 2910 and the hub adapter 8004, and B_{z} is the magnetic field strength in the direction of axial movement of the hub 2910. In some embodiments, Δz is proportional to B_{z}.

In part, the movement of the hub adapter can result in a relative displacement in the z-direction between a hub and a hub adapter, as mentioned above. This relative displacement can lead to a magnetic coupling force that can move the hub toward the hub adapter in the z-direction. In other cases, external forces can act on the hub and the device attached to the hub, which can result in a relative displacement in the z-direction between the hub and the hub adapter. In those cases, the magnetic coupling force can increase to balance out the external forces. But, in such cases where there is an external force applied to the hub, there can be a relative displacement in the z-direction between the hub and the hub adapter due to the external forces. In some embodiments, the amount of the relative displacement will depend on a magnitude of the external force, inter alia.

Note also that, even though FIG. 24 shows magnets coupled with both the hub 2910 and the hub adapter 8004, some embodiments of the system can be configured such that only the hub 2910 includes a magnet. The hub adapter 8004 can support the sensor (which can be a magnetometer, as discussed), and can be configured to be coupled with the hub without having a second magnet in the hub adapter 8004.

In some embodiments, the hub adapter 8004 and hub 2910 can be configured such that the hub 2910 can withstand a force (e.g., in an axial direction) of up to 10 N, up to approximately 10 N, up to 15 N, up to approximately 15N, up to 20 Newton, up to approximately 20 N, from 10 N to 30N, from approximately 10 N to approximately 30 N, from 15 N to 25 N, from approximately 15 N to approximately 25 N, or any other suitable force or range of forces before detaching from the hub adapter 8004.

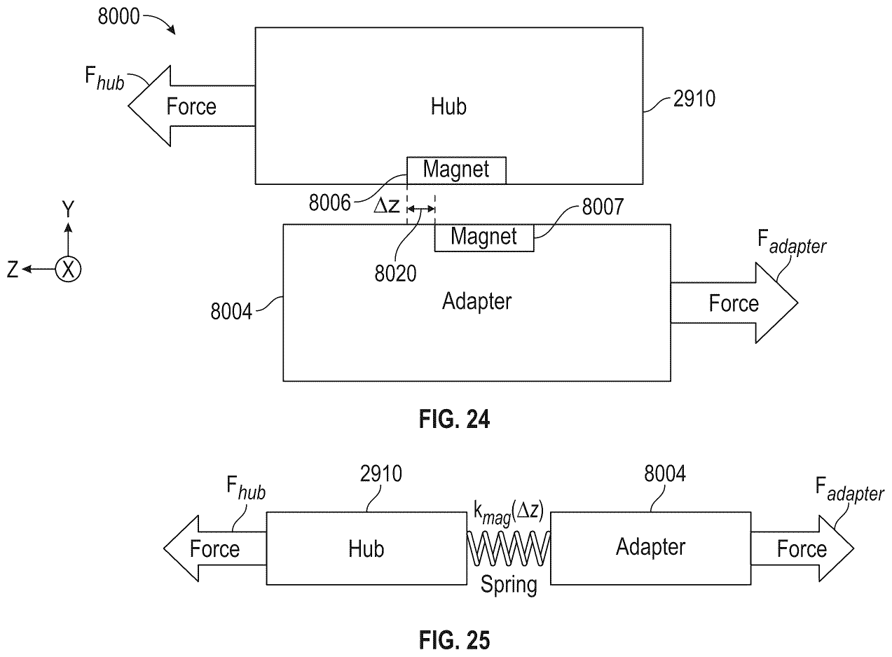

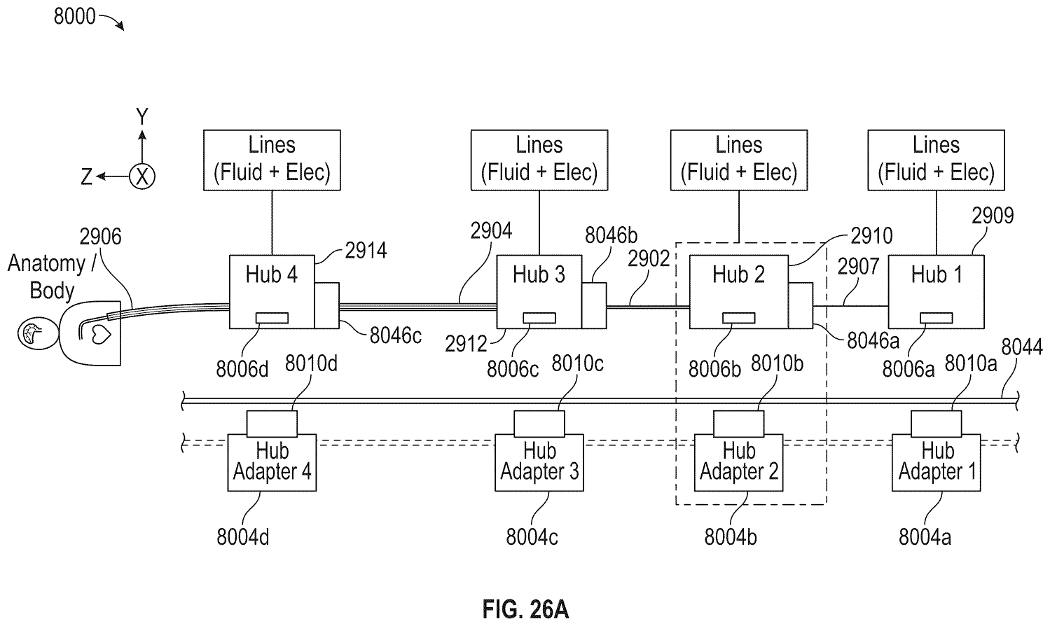

While a hub 2910 and hub adapter 8004 are described with respect to FIGS. 24 and 25, it will be understood by one of skill in the art that any of the hubs described herein (e.g., hub 2909, hub 2912, and hub 2914) and corresponding hub adapters (e.g., hub adapter 8004a, hub adapter 8004c, and hub adapter 8004d, as shown in FIG. 26A) may operate in the same or a similar manner.

In some embodiments, different interventional devices and/or different hubs can be configured to detach from the respective hub adapters at different magnitudes of force (e.g., can have different threshold shearing forces).

In any embodiments disclosed herein, the robotic control system can include additional electronic components to accompany the magnetometer, including without limitation, a microcontroller (which can be an Arduino-IDE programmable board), a power supply for the magnetometer and other components, memory storage devices, one or more wired or wireless communication devices to communicate with the magnetometer, a display device, directly to a workstation, or to any other desired components, and/or a data processor configured to receive data from the magnetometer and to perform calculations based on the data from the magnetometer.

Some embodiments of the system can have a processor (e.g., a separate, standalone computer) configured to be in communication with one or more sensors of the system. The processor can be configured to transmit sensor data from a second processor of the system. In some embodiments, the system can be configured to have only a single processor.

Some embodiments will be configured to execute programs and have the appropriate software to execute at least the functions disclosed herein. For example and without limitation, some embodiments of the system disclosed herein can be configured to have and execute a noise reduction algorithm, an algorithm configured to determine a position of the hub adapter and/or hub in the z-direction (for any of the hubs and/or hub adapters of the system), an algorithm configured to compute relative displacement in the z-direction between a hub and a hub adapter based on raw magnetic field vector data (for any of the hubs of the system), an algorithm configured to compute external forces on a hub (e.g., based on raw magnetic field vector data) (for any of the hubs of the system), an algorithm configured to compute a coupling force between a hub and a hub adapter (e.g., based on raw magnetic field vector data), algorithms configured to provide alerts or warnings to a user or otherwise when threshold values or conditions are reached (such as relative displacement or external force threshold values), and/or algorithms configured to process data and/or data signals into a user-friendly format for displaying on a graphical user interface. Any embodiments of the system disclosed herein can have a controller programmed to perform any or any combination of the foregoing algorithms.

Some embodiments of the robotic control system can have signal boosters and/or noise reduction or noise rejection components to improve the data from the sensors. Additionally, a main computer or computing device can be configured to communicate with each of the sensors in the robotic control system (including the magnetometers) and to output such data from the sensors to a user display, a memory storage device, and/or otherwise.

In some embodiments, the robotic control system can be configured to provide a warning to a user when axial torque is above a threshold at the proximal end of one or more interventional devices. In some embodiments, such axial torque can be calculated by collecting data related to the reaction torque on the end of the interventional device (e.g., insert or access catheter) or the motor drive mechanism using a torque sensor, as an example.

FIG. 26A shows a schematic illustration of an example embodiment of a portion of a robotic control system 8000 showing the guidewire hub 2909, the access catheter hub 2910, the procedure catheter hub 2912, and the guide catheter hub 2914 respectively magnetically coupled to a plurality of hub adapters 8004a, 8004b, 8004c, and 8004d. As shown in FIGS. 26A, the hubs are separated from the hub adapters by a sterile barrier 8044.

As shown in FIG. 26A, the hub 2910 can be coupled to or include a hemostasis valve 8046a to accommodate introduction of the guidewire 2907 therethrough. The hub 2912 can be coupled to or include a hemostasis valve 8046b to accommodate introduction of the access catheter 2902 and/or guidewire 2907 therethrough. The hub 2914 can be coupled to or include a hemostasis valve 8046c to accommodate introduction of the procedure catheter 2904, access catheter 2902, and/or guidewire 2907 therethrough.

Any of the hubs of any embodiments disclosed herein, including without limitation the first hub 2909, second hub 2910, third hub 2912, and/or fourth hub 2914 shown in FIG. 26A, can have one or more magnets coupled therewith, such as and without limitation, magnets 8006a, 8006b, 8006c, and 8006d, respectively. In some embodiments, a magnetic field can be created between the hub adapter and the hub using the one or more magnets on each hub and hub adapter pair, the system being configured such that a movement of the hub adapter results in a movement of the hub as a result of the magnetic field between the hub and hub. Magnetic coupling can be used in any embodiments of the robotic control systems disclosed herein so that a movement of a driving device on a non-sterile side of a sterile barrier can cause a movement of at least one device on the sterile side of the sterile barrier in any desired direction of movement (e.g., in the direction of insertion and/or withdrawal of an interventional device relative to a port going into a patient's body).

While four hubs and four hub adapters are shown in FIG. 26A, any embodiments of the robotic control system disclosed herein can have one, two, three, five, or more of each of hubs and hub adapters, in pairs or not in pairs. In other words, some embodiments of the robotic control system disclosed herein can have more hubs than hub adapters.

In some embodiments, each hub and hub adapter pair can have a sensor. As shown in FIG. 26A, the robotic control system 8000 can have a plurality of sensors 8010a, 8010b, 8010c, and/or 8010d. In any embodiments disclosed herein, the “sensor” of each pair of hub and hub adapter can include multiple sensors. For example and without limitation, the multiple sensors can include any combination of the following: one or more magnetic field sensors, one or more magnetic inductance sensors, one or more capacitance sensors, one or more magnetic optical sensors, one or more general force sensors, one or more magnetic force sensors, and/or one or more magnetic load sensors (for example and without limitation, current sensors configured to determine motor load values of any drive components for the hub adapters, such as motors used for the rack and pinion or other linear actuation devices). Such sensors can be or can have any of the details of any of the sensors disclosed herein or that are known in the art. In some embodiments, each hub and hub adapter pair or any of the hub and hub adapter pairs of any embodiments of the robotic control system disclosed herein can include multiple redundant sensors such as a magnetic field sensor (for example and without limitation, a magnetometer) and another secondary sensor. Any of the sensors disclosed herein can be configured to determine one or more of the following parameters or any combination or all of the following parameters: relative displacement between a hub and a corresponding hub adapter, a direction of displacement of a hub relative to a corresponding hub adapter, a net force applied to a hub and/or a hub adapter, and/or a net force vector applied to a hub and/or a hub adapter. In some embodiments, the secondary sensor can be any of the sensor types disclosed herein. In some embodiments, the secondary sensor can be used to gather redundant or additional, unique data.

As mentioned, some embodiments of the robotic control system can have an inductance sensor (for example, an inductance position sensor). In some embodiments of the robotic control system wherein the hub and hub adapter pair have an inductance sensor, the sensor can be positioned on one or the other of the hub and hub adapter and can be configured to emit an electromagnetic field from a face of the sensor. For example and without limitation, the inductance sensor can be positioned on the hub adapter and can have a main surface configured to face the hub from which the electromagnetic field can be emitted. A metal target can be positioned in a designated position on the other of the hub and the hub adapter, in this nonlimiting example, on the hub. The metal target can be configured to disrupt the electromagnetic field, which can be detected by the sensor. In some embodiments, the target metal can have any shape or size suitable for positioning on the hub or hub adapter. In some embodiments, the target metal will be positioned on the hub.

The position of the sensor and the metal target on the hub and hub adapter will be known. With this information and based on the parameters of the magnetic field that the sensor can detect and gather, the system can determine the existence of an offset between the hub and hub adapter and, if applicable, a relative offset distance between the hub and hub adapter. In any embodiments, the inductance sensor can be shielded or unshielded, normally open or normally closed, an NPN configuration (positively doped semiconducting agent positioned between two negatively doped materials), or a PNP configuration (negatively doped semiconducting agent positioned between two positively doped materials), and/or have any other details or features known in the industry or to one of ordinary skill in the art. In some embodiments, the inductance sensor can include one or more coils or antennas along with a microcontroller unit and/or other processing electronics.

As mentioned, in some embodiments, any of the sensors 8010a-d can include or can be a magnetic sensor, such as a magnetometer. In some embodiments, any of the sensors 8010a-d can be configured to measure a range of parameters, including but not limited to magnetic field strength and/or direction from one or more magnets of the robotic control system (e.g., magnets 8006a-d). For example, each sensor 8010a-d can be configured to measure a magnetic field strength and/or direction from a respective magnet 8006a-d within the pair of the hub and hub adapters that the sensor 8010a-d is part of.

Each of the sensors 8010a-d can be configured to determine relative displacement or offset between a corresponding pair of hubs and hub adapters (e.g., hub 2909 and hub adapter 8004a, hub 2910 and hub adapter 8004b, hub 2912 and hub adapter 8004c, and hub 2914 and hub adapter 8004d). Additionally or alternatively, any of the sensors 8010a-d can be configured to determine the magnitude of forces being applied to a corresponding hub 2909, 2910, 2912, and 2914, as discussed above, based on magnetic field strength and/or direction from the respective magnet 8006a-d.

In some embodiments, any of the sensors 8010a-d can be a tri-axis magnetometer. In some embodiments, such a magnetometer can be configured to detect a strength of a magnetic field in x, y, and z directions. In some embodiments, the magnetic field strength and/or direction values can be used to determine relative displacement between a corresponding hub and hub adapter pair (e.g., hub 2909 and hub adapter 8004a, hub 2910 and hub adapter 8004b, hub 2912 and hub adapter 8004c, and hub 2914 and hub adapter 8004d). In some embodiments, the magnetic field strength and/or direction values can be used to determine a force on the magnetic coupling between a corresponding hub and hub adapter pair (e.g., hub 2909 and hub adapter 8004a, hub 2910 and hub adapter 8004b, hub 2912 and hub adapter 8004c, and hub 2914 and hub adapter 8004d). In some embodiments, the magnetic field strength and/or direction values can be used to determine a force acting on the corresponding hub.

In some embodiments, the magnetometer can have a 16-bit output proportional to the magnetic flux density sensed along the XYZ axis and also, in some embodiments, a temperature output signal. Any of the sensors 8010a-d can be configured to provide digital values via PC and SPI, where the sensor 8010a-d is a slave on the bus. In some embodiments, any of the sensors 8010a-d can be programmed to have any of a desired range of duty cycles, from for example and without limitation, a range of 0.1% to 100%. In some embodiments, any of the sensors 8010a-d can be configured to only acquire and provide force and/or displacement information to a user when a particular threshold of force and/or displacement are detected by the sensor 8010a-d.

In some embodiments, any of the sensors 8010a-d can be positioned at or coupled to a portion of a corresponding hub adapter 8004a-d (e.g., a center portion). A corresponding magnet 8006a-d (such as, for example, a strong magnet or a rare earth magnet) can be positioned at or coupled to a portion of a corresponding hub 2909, 2910, 2912, or 2914 (e.g., a center portion). In some embodiments, a center or center portion of the magnet 8006a-d can align or approximately align with a center portion of a corresponding sensor 8010a-d. Displacement can be determined based on an offset between the center portion of a magnet 8006a-d and the center portion of the corresponding sensor 8010a-d.

FIG. 26B illustrates an example embodiment of a magnetically coupled hub adapter 8004b and hub 2910 that can be used with any embodiments of the robotic control system disclosed herein, including the robotic control system 8000 or portion of the embodiment of the robotic control system that is shown in FIG. 26A. FIG. 26B schematically illustrates a range of different forces that can be applied to the hub and corresponding hub adapter. In the example shown in FIGS. 26B, a range of different forces that can be applied to the hub 2910 and hub adapter 8004b are shown. However, it will be appreciated by one of skill in the art that any of the same or similar types of forces may be applied to any of the other hubs described herein (e.g., hubs 2909, 2912, and 2914).

For example, in some arrangements and in some applications, forces that can act on a hub (e.g., hub 2910 in FIG. 26B) can include: forces from the body (F_{body}) acting on the interventional device coupled with the hub (e.g., the access catheter 2902 in FIG. 26B), forces from the coupled catheter (e.g., the access catheter 2902 in FIG. 26B) or other interventional device (F_{catheter}) that can act on the hub, forces from a distal hemostasis valve (F_{THV}) (e.g., hemostasis valve 8046b with respect to hub 2910) that can act on the interventional device coupled with the hub, distal anti-buckling forces (F_{anti-buckling}) that can act on the interventional device coupled with the hub, forces acting on the hub from hub bearings or rollers in the z direction (F_{bearing z}) such as friction, inertial forces, and other resistance forces, forces from a proximal hemostasis valve (F_{THV 2}) (e.g., hemostasis valve 8046a with respect to hub 2910) acting on the hub, such as from a proximal instrument passing through the proximal hemostasis valve (e.g., from the guidewire 2907 with respect to hub 2910), proximal anti-buckling forces (F_{anti-buckling 2}) that can act on the hub, gravitational forces based on the mass of the hub (F_{g}), a force from the magnetic field in the y direction (F_{magnet y}), a force from the magnetic field in the z direction (F_{magnet z}), forces acting on the hub from the bearings or rollers in the y direction (F_{bearing y}), and/or forces acting on the hub from the electrical and/or fluid lines (Fₗᵢₙₑₛ), which can act in the z direction, the y direction, and/or the x direction, and any combination of these directions. F_{catheter} can represent friction forces between the device attached to the hub and other devices in contact with the device. For example and without limitation, F_{catheter} can include friction forces from interaction between a catheter and a hub that the catheter is passing through, or friction forces from interaction of a procedure catheter and a guide catheter. F_{body} can represent the net friction between the walls of the vasculature and the device attached to the hub.

In some embodiments, anti-buckling support mechanisms, such as telescoping tubes, may be provided. The use of these can result in drag forces on the catheter or guidewire, and/or insertion or retraction forces from a rubbing of an outer surface of the shafts against an inner surface of the anti-buckling support mechanisms. In some embodiments, there can be a distinction between which parts of any anti-buckling system apply external loads to a hub. When a device is loaded in compression and buckles, there can be contact between the device and the inner walls of the anti-buckling component (e.g., a split tube or telescoping tubes in some embodiments). F_{anti-buckling} can represent the forces transmitted through fasteners/interlocking features that attach the anti-buckling components to the device hub and/or friction between the device and sections of an anti-buckling device, such as the walls of telescoping tubes.

Fᵣₑₜₐᵢₙₑᵣ can represent the downward, retaining force that prevents the magnets in the hub adapter from being pulled vertically into the sterile barrier. On the free-body diagram, it opposes the vertical lift force from the magnetic coupling, F_{magnet, y}. The downward, retaining force can be provided by any suitable retaining mechanism, such as a screw or other fastener.

In some arrangements and in some applications, forces that can act on a hub adapter (e.g., hub adapter 8004b in FIG. 26B) can include without limitation, a force from the magnetic field in the y direction (F_{magnet y}), a force from the magnetic field in the z direction (F_{magnet z}), a force exerted on the hub adapter from the drive system (F_{drive}), and/or a gravitational force from the mass of the hub adapter (F_{g adapter}).

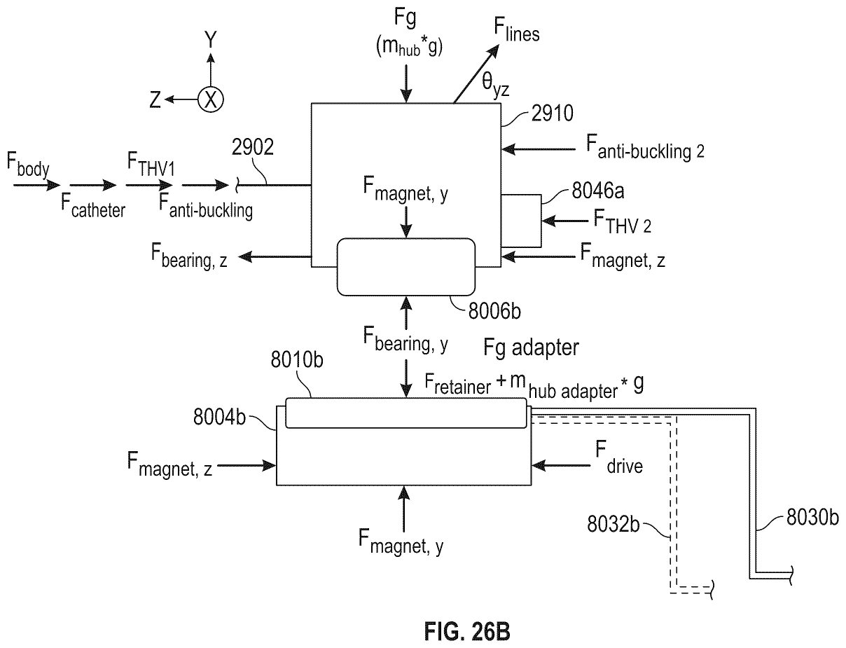

FIG. 27 illustrates a wiring schematic that can be used with any embodiment of the robotic control system 8000 disclosed herein. As described above, some embodiments of the robotic control system 8000 can have four (or more) hubs (e.g., hubs 2909, 2910, 2912, and 2914) and four (or more) paired hub adapters (e.g., hub adapters 8004a, 8004b, 8004c, and 8004d), each having a sensor (e.g., sensors 8010a, 8010b, 8010c, and 8010d), and any of the other components or features disclosed herein. Each sensor 8010a-d can be a magnetometer. Each sensor 8010a-d can have corresponding power and ground wires 8030a-d, and inter-integrated-circuit (I²C) communication wires 8032a-d coupled with each sensor 8010a-d and with a microcontroller 8038. A signal booster 8040 can be used to increase an amplitude of the signals from the sensors 8010a-d through at least the communication wires 8032a-d. Additionally, in some embodiments, the booster can split the I2C signal into a differential I2C signal, reducing noise along a long length of wiring. A USB or other communication wire 8042 can be coupled with the microcontroller 8038, to provide a communication link with a main controller, a computer, an input device, or otherwise.

Note that the hubs are also referred to as pucks herein. The use of the term hub herein is meant to be synonymous with the term puck. Therefore, any use of the term hub or puck is meant to be used interchangeably and to refer to the same components. Note that the hub adapters are also referred to as carriages herein. The use of the term hub adapter is meant to be synonymous with the term carriage. Therefore, any use of the term hub adapter or carriage is meant to be used interchangeably and to refer to the same components.

While certain embodiments of the inventions have been described, these embodiments have been presented by way of example only, and are not intended to limit the scope of the disclosure. Indeed, the novel methods and systems described herein may be embodied in a variety of other forms. Furthermore, various omissions, substitutions and changes in the robotic drive systems and methods described herein may be made without departing from the spirit of the disclosure. The accompanying claims and their equivalents are intended to cover such forms or modifications as would fall within the scope and spirit of the disclosure. Accordingly, the scope of the present inventions is defined only by reference to the appended claims.

Features, materials, characteristics, or groups described in conjunction with a particular aspect, embodiment, or example are to be understood to be applicable to any other aspect, embodiment or example described in this section or elsewhere in this specification unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing embodiments. The protection extends to any novel one, or any novel combination, of the features disclosed in this specification (including any accompanying claims, abstract and drawings), or to any novel one, or any novel combination, of the steps of any method or process so disclosed.

Furthermore, certain features that are described in this disclosure in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations, one or more features from a claimed combination can, in some cases, be excised from the combination, and the combination may be claimed as a subcombination or variation of a subcombination.

Moreover, while operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate that in some embodiments, the actual steps taken in the processes illustrated and/or disclosed may differ from those shown in the figures. Depending on the embodiment, certain of the steps described above may be removed, others may be added. Furthermore, the features and attributes of the specific embodiments disclosed above may be combined in different ways to form additional embodiments, all of which fall within the scope of the present disclosure. Also, the separation of various system components in the implementations described above should not be understood as requiring such separation in all implementations, and it should be understood that the described components and systems can generally be integrated together in a single product or packaged into multiple products.

For purposes of this disclosure, certain aspects, advantages, and novel features are described herein. Not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein.

Conditional language, such as “can,” “could,” “might,” or “may,” unless specifically stated otherwise, or otherwise understood within the context as used, is generally intended to convey that certain embodiments include, while other embodiments do not include, certain features, elements, and/or steps. Thus, such conditional language is not generally intended to imply that features, elements, and/or steps are in any way required for one or more embodiments or that one or more embodiments necessarily include logic for deciding, with or without user input or prompting, whether these features, elements, and/or steps are included or are to be performed in any particular embodiment.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount. As another example, in certain embodiments, the terms “generally parallel” and “substantially parallel” refer to a value, amount, or characteristic that departs from exactly parallel by less than or equal to 15 degrees, 10 degrees, 5 degrees, 3 degrees, 1 degree, or 0.1 degree. The ranges disclosed herein also encompass any and all overlap, sub-ranges, and combinations thereof, and any specific values within those ranges. Language such as “up to,” “at least,” “greater than,” “less than,” “between,” and the like includes the number recited. Numbers and values used herein preceded by a term such as “about” or “approximately” include the recited numbers. For example, “approximately 7 mm” includes “7 mm” and numbers and ranges preceded by a term such as “about” or “approximately” should be interpreted as disclosing numbers and ranges with or without such a term in front of the number or value such that this application supports claiming the numbers, values and ranges disclosed in the specification and/or claims with or without the term such as “about” or “approximately” before such numbers, values or ranges such, for example, that “approximately two times to approximately five times” also includes the disclosure of the range of “two times to five times.” The scope of the present disclosure is not intended to be limited by the specific disclosures of preferred embodiments in this section or elsewhere in this specification, and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

1. A robotic control system, comprising:
a hub configured to adjust an axial position of an interventional device;
a driven magnet coupled with the hub;
a hub adapter configured to move in at least one direction based on an input provided by a user of the robotic control system;
a drive magnet coupled with the hub adapter and configured to magnetically couple with the driven magnet in an operable state of the robotic control system such that the driven magnet moves in response to a movement of the drive magnet; and
a sensor coupled with the hub or the hub adapter and configured to measure a magnitude of a magnetic field on the sensor.

2. (canceled)

3. (canceled)

4. The robotic control system of claim 1, wherein the sensor is a magnetometer.

5. (canceled)

6. The robotic control system of claim 1, wherein the robotic control system is configured to determine a relative distance in the at least one direction between the hub adapter and the hub when the hub is at least partially offset from the hub adapter in the at least one direction based on the measured magnitude of the magnetic field on the sensor.

7. (canceled)

8. (canceled)

9. (canceled)

10. The robotic control system of claim 1, wherein the robotic control system is configured to determine a magnitude of a net external force acting on the hub in the at least one direction based on the measured magnitude of the magnetic field on the sensor.

11. The robotic control system of claim 10, wherein the robotic control system is configured to provide an alert when the net external force in the at least one direction reaches or exceeds a threshold value.

12. The robotic control system of claim 10, wherein the robotic control system is configured to automatically implement a correction action when the net external force in the at least one direction reaches or exceeds a threshold value.

13. (canceled)

14. (canceled)

15. The robotic control system of claim 1, wherein the robotic control system is configured to determine a relative distance in the at least one direction between the hub adapter and the hub when the hub is at least partially offset from the hub adapter in the at least one direction based on the measured magnitude of the magnetic field on the sensor, and/or wherein the robotic control system is configured to determine a magnitude of a net external force acting on the hub in the at least one direction based at least in part on the relative distance in the at least one direction between the hub adapter and the hub when the hub is offset from the hub adapter in the at least one direction.

16. The robotic control system of claim 1, wherein the hub is coupled to the hub adapter across a sterile barrier, wherein the hub is positioned on a sterile side of the sterile barrier and the hub adapter is positioned on a nonsterile side of the sterile barrier.

17. (canceled)

18. (canceled)

19. (canceled)

20. (canceled)

21. (canceled)

22. The robotic control system of claim 1, further comprising:
a second hub configured to adjust an axial position of a second interventional device;
a second driven magnet coupled to the second hub;
a second hub adapter configured to move in the at least one direction based on an input provided by the user of the robotic control system;
a second drive magnet coupled with the second hub adapter and configured to couple with the second driven magnet such that the second driven magnet moves in response to movement of the second drive magnet; and
a second sensor coupled with the second hub or the second hub adapter and configured to measure a magnitude of a second magnetic field.

23. (canceled)

24. (canceled)

25. (canceled)

26. (canceled)

27. (canceled)

28. (canceled)

29. (canceled)

30. (canceled)

31. (canceled)

32. The robotic control system of claim 1, comprising at least four hub adapters each having a magnet and four corresponding hubs, wherein each of the four hub adapters has a sensor configured to measure a magnitude of a magnetic field on the sensor from the magnet of each of the corresponding hubs.

33. (canceled)

34. (canceled)

35. (canceled)

36. (canceled)

37. (canceled)

38. The robotic control system of claim 1, comprising a ring magnet coupled with the hub and/or the hub adapter, the ring magnet positioned and configured to provide the magnetic field on the sensor.

39. The robotic control system of claim 1, comprising wherein the sensor is configured to also measure a direction of the magnetic field on the sensor so that the robotic control system can determine a magnitude and a direction of a displacement of the hub relative to the hub adapter.

40. The robotic control system of claim 1, further comprising a controller configured to determine a magnitude of a net external force acting on the hub in the at least one direction based on the measured magnitude of the magnetic field on the sensor;
wherein the robotic control system is configured to output a warning to the user of the robotic control system when the magnitude of the net external force acting on the hub in the at least one direction reaches a threshold value comprising a predetermined percentage of a breakaway force.

41. A method of controlling a movement of an interventional device through a sterile barrier, comprising:
magnetically coupling a hub on a sterile side of the sterile barrier with a hub adapter on a non-sterile side of the sterile barrier such that the hub moves in response to a movement of the hub adapter;
moving the hub in at least one direction by moving the hub adapter in the at least one direction while the hub is biased to remain in alignment with the hub adapter in the at least one direction by the magnetic coupling; and
determining a distance between the hub and the hub adapter and/or a net external force applied to the hub when the hub is offset in the at least one direction from the hub adapter based on measurement of a magnetic field on a sensor coupled to the hub or the hub adapter.

42. The method of claim 41, wherein the hub is coupled to an interventional device, wherein the interventional device is a guide catheter, a procedure catheter, an access catheter, or a guidewire.

43. (canceled)

44. The method of claim 41, comprising determining a magnitude of an external force acting on the hub in the at least one direction when the hub is offset in the at least one direction from the hub adapter.

45. (canceled)

46. The method of claim 41, comprising:
magnetically coupling a second hub on the sterile side of the sterile barrier with a second hub adapter on the non-sterile side of the sterile barrier such that the second hub moves in response to movement of the second hub adapter;
moving the second hub in the at least one direction by moving the second hub adapter in the at least one direction while the second hub is biased to remain in alignment with the second hub adapter in the at least one direction by the magnetic coupling between the second hub adapter and the second hub; and
determining a second distance between the second hub and the second hub adapter and/or a second net external force applied to the second hub when the second hub is offset in the at least one direction from the second hub adapter based on measurement of a magnetic field from the magnetic coupling on a sensor coupled to the second hub or the second hub adapter.

47. (canceled)

48. (canceled)

49. (canceled)

50. (canceled)

51. (canceled)

52. The method of claim 41, further comprising outputting a warning when a magnitude of the net external force acting on the hub in the at least one direction reaches a threshold value comprising a predetermined percentage of a breakaway force.

53. (canceled)

54. (canceled)

55. The method of claim 41, comprising impeding the movement of the hub adapter in a direction that would increase the net external force acting on the hub when the net external force acting on the hub reaches a threshold value.

56. The method of claim 41, comprising automatically implementing a correction action when the net external force in the at least one direction reaches or exceeds a threshold value.

57. (canceled)

58. (canceled)

59. (canceled)

60. (canceled)

61. (canceled)

62. (canceled)

63. (canceled)

64. (canceled)

65. (canceled)

66. (canceled)
